(19) United States
(12) Patent Application Publication
(10) Pub. No.: US 2021/0387156 A1
(43) Pub. Date: Dec. 16, 2021

(54) METHOD FOR PRODUCING MICROPARTICLES WHICH ARE CHARGED WITH AN ACTIVE MATERIAL

(71) Applicant: BASF SE, Ludwigshafen am Rhein (DE)

(72) Inventors: Bernd Dieter OSCHMANN, Ludwigshafen am Rhein (DE); Kerstin MUELHEIMS, Ludwigshafen am Rhein (DE); Wolfgang KRAUSE, Lampertheim (DE); Patrick LEIBACH, Ludwigshafen am Rhein (DE); Roland Hinrich STAFF, Ludwigshafen am Rhein (DE); Dieter FLICK, Ludwigshafen am Rhein (DE); Lukas MAYR, Ludwigshafen am Rhein (DE); Stephanie RENZ, Ludwigshafen am Rhein (DE); Felicitas GUTH, Ludwigshafen am Rhein (DE); Klaus KAHLE, Ludwigshafen am Rhein (DE); Stephan HUEFFER, Ludwigshafen am Rhein (DE); Tobias Maximilian MERKEL, Ludwigshafen am Rhein (DE); Axel BINDER, Ludwigshafen am Rhein (DE); Karl KOLTER, Ludwigshafen am Rhein (DE); Yean Yik GEOERG, Ludwigshafen am Rhein (DE); Regina VOGELSANG, Ludwigshafen am Rhein (DE); Antoine Maxime Charles Jos BEZIAU, Ludwigshafen am Rhein (DE)

(21) Appl. No.: 17/290,669

(22) PCT Filed: Oct. 29, 2019
(86) PCT No.: PCT/EP2019/079449

§ 371 Date: Apr. 30, 2021

(30) Foreign Application Priority Data
Oct. 30, 2018 (EP) 18203460.3
Apr. 4, 2019 (EP) 19167363.1

(51) Int. Cl. B01J 13/20; B01J 13/22

(52) U.S. Cl. CPC B01J 13/203; B01J 13/22

(57) ABSTRACT

The present invention relates to processes for producing microparticles having, in their interior, at least one cavity which is connected via pores to the surface of the microparticles and which have been laden with at least one organic active of low molecular weight. The invention especially relates to a process for loading microparticles with at least one organic active of low molecular weight, wherein the active has been embedded in a matrix and/or the pores of the microparticles have been closed by means of a substance applied to the surface of the microparticles. The invention additionally relates to a process for sealing microparticles laden with at least one organic active of low molecular weight. The invention also relates to compositions of microparticles laden with at least one active of low molecular weight and to the use thereof.

The present invention relates to processes for producing microparticles laden with at least one organic active of low molecular weight, especially to a process for loading microparticles with at least one organic active of low molecular weight and to a process for sealing microparticles laden with at least one organic active of low molecular weight. The invention also relates to compositions of microparticles laden with at least one active of low molecular weight and to the use thereof.

Microcapsules have various different uses as carriers for active substances, for example for crop protecting agents, pharmaceutical agents, fragrances and aromas, but also for reactive substances or catalysts for industrial applications. They typically comprise a polymer material that envelops the material to be encapsulated. Advantages of a formulation of this kind are in particular:
protection of reactive actives from environmental effects;
safe and practical handling of toxic or unstable actives;
controlled release of actives;
prevention of premature mixing of substances;
the handling of liquid actives as solids.

An overview of the methodology of microencapsulation of actives can be found in H. Mollet, A. Grubenmann, “Formulation Technology”, chapter 6.4 (Microencapsulation), Wiley VCH Verlag GmbH, Weinheim 2001, and the literature cited therein.

There have been a variety of descriptions of porous microparticles that consist of a polymer material in spongelike form and can be laden with active ingredients.

EP 467528 describes porous polymeric carrier particles with average particle sizes up to 250 μm and pores on their surface. The porous polymeric carrier particles are produced by suspension polymerization of styrene and a polyester of maleic anhydride/phthalic anhydride/propylene glycol in the presence of pore-forming substances. The particles are proposed for enzymes, catalysts and bacteria.

WO 2011/088229 describes porous microparticles composed of biodegradable polymers, e.g. poly(lactide-co-glycolide) (PLGA), comprising, in the pores, an ionic species, e.g. an inorganic salt of a polyvalent ion, which is capable of binding to the active. After the loading with the active, which is generally a biologically active polymer, e.g. a protein, a lipoprotein, a proteoglycan or a nucleic acid, the pores are sealed by heating.

WO 2015/070172 describes porous microparticles composed of biodegradable polymers wherein the pores comprise, as active, a biologically active polymer, e.g. a protein, a lipoprotein, a proteoglycan or a nucleic acid, and an ionic biopolymer, especially an ionic polysaccharide, and a pH modifier, e.g. magnesium carbonate or zinc carbonate. The ionic biopolymer forms an ionic complex with the biologically active polymer. After the microparticles have been laden with a biologically active polymer, e.g. a protein, a lipoprotein, a proteoglycan or a nucleic acid, the pores are sealed by heating.

The methods described in the aforementioned prior art documents teach the loading of porous microparticles with biologically active polymers that are to be released rapidly at the site of use. The continuous release of the active over a prolonged period is of no significance here. Indeed, such a release is not wanted. Moreover, biologically active polymers are very hydrophilic. There is no description of the loading of the porous microparticles with substances of low molecular weight or even hydrophobic substances such as aroma chemicals or crop protecting agents. In all processes, the laden microparticles, after the loading, are heated fora prolonged period of generally several hours up to several days in order to close the pores and to prevent premature exit of the active. This regularly leads to stress on the active ingredient and can lead to unwanted degradation of the active. Moreover, the production of the microparticles is complex since the porous microparticles, in the course of production, generally have to be treated with a substance that binds the actual active.

WO 2018/065481 describes a process for filling porous microparticles with an aroma chemical by suspending the microparticles in a liquid aroma chemical or solution of the aroma chemical. Here too, the sealing is effected by heating, which can lead to degradation of the active. Moreover, the release characteristics are not always satisfactory.

It is therefore an object of the invention to provide a process for loading porous microparticles with actives in which thermal treatment of the laden microparticles can be avoided. The laden microparticles are to release the active only after a period of latency. More particularly, it is desirable to achieve controlled release of the active. For example, it may be desirable for the release rate to be very substantially constant over a prolonged period. In other cases, it is desirable to achieve rapid release of the active in a controlled manner after the period of latency. The laden microparticles were to be producible in a simple process and to be inert toward the active.

It has been found that, surprisingly, these and further objects are achieved by the processes described hereinafter for loading porous microparticles, i.e. microparticles having pores on their surface, with active and the active-filled microparticles that are obtainable thereby.

The present invention therefore relates to a process for producing microparticles laden with at least one organic active of low molecular weight, wherein the microparticles have been formed from an organic, polymeric wall material and in the unladen state have at least one cavity in their interior which is connected via pores to the surface of the microparticles, wherein one of the following measures (a), (b), (c) and (d) is taken:

### Measure (a):

The unladen microparticles are impregnated with a liquid (1a) consisting essentially of:
i) the organic active of low molecular weight, in molten, emulsified, suspended or dissolved form in the liquid,
ii) at least one nonpolymerizable substance A which is solid at room temperature and is in molten, emulsified, suspended or dissolved form in the liquid, and
iii) optionally one or more solvents,
   wherein any solvent present thereafter is optionally but not necessarily removed;

### Measure (b):

The unladen microparticles are impregnated with a liquid (1b) consisting essentially of:
i) the organic active of low molecular weight, in molten, emulsified, suspended or dissolved form in the liquid,
ii) at least one polymerizable substance B in emulsified or dissolved form in the liquid,
iii) optionally a nonpolymerizable substance A which is solid at room temperature and is in molten, emulsified, suspended or dissolved form in the liquid, and
iv) optionally one or more solvents,
   wherein polymerization of substance B is subsequently brought about and any solvent present is optionally but not necessarily removed;

### Measure (c):

The unladen microparticles are impregnated with a liquid (1c) consisting essentially of:
i) the organic active of low molecular weight, in molten, emulsified, suspended or dissolved form in the liquid,
ii) at least one substance C which is in dissolved or molten form in the liquid and can be solidified by addition of polyvalent ions,
iii) optionally a nonpolymerizable substance A which is solid at room temperature and is in molten, emulsified, suspended or dissolved form in the liquid, and
iv) optionally one or more solvents,
   wherein a solution of polyvalent ions is subsequently added in order to bring about solidification, i.e. precipitation, of substance C and any solvent present is optionally but not necessarily removed;

### Measure (d):

A substance that seals the pores of the microparticles is applied to the surface of the microparticles that have already been laden with at least one organic active of low molecular weight. The unladen microparticles are laden with the at least one organic active of low molecular weight by impregnating the microparticles with a liquid (1d) comprising the active.

Measures (a), (b), (c) and (d) enclose the active in the microparticles after the filling. In this case, substances (A), (B) and (C) used in measures (a), (b) and (c)—in the case of substances (B) and (C) after they have been solidified by polymerization or by treatment with the polyvalent metal ions—form a solid matrix that encloses the active. In the case of measure (d), the enclosure is achieved by sealing of the pores with a substance applied to the surface of the pores, especially by the production of a solid layer on the surface of the laden microparticles, which leads to sealing of the pores.

Accordingly, the present invention also relates to a process for sealing microparticles laden with at least one organic active of low molecular weight, wherein the microparticles have been formed from an organic, polymeric wall material and, in the unladen state, in their interior, have at least one cavity connected via pores to the surface of the microparticles, wherein at least one substance that seals the pores of the laden microparticles is applied to the surface of the microparticles laden with the organic active of low molecular weight, especially by producing a solid coating on the surface of the laden microparticles.

The present invention further relates to compositions of microparticles filled with at least one active, obtainable by a process of the invention, and to the use thereof, especially in a product selected from perfumes, washing and cleaning compositions, cosmetic compositions, personal care compositions, hygiene articles, foods, food supplements, fragrance dispensers and fragrances.

The present invention further relates to products comprising an inventive composition of microparticles filled with at least one active, and to the use thereof, especially for controlled release of actives of low molecular weight and specifically for controlled release of aroma chemicals.

It has also been found that, by contrast to the description in the prior art, impregnation of the unladen microparticles with the active does not require the unladen microparticles to be suspended in a liquid comprising the active. Instead, it is possible to impregnate the unladen microparticles by other, different methods. In particular, it is possible to achieve impregnation of the unladen microparticles in an efficient manner by applying a liquid comprising the active, for example the aforementioned liquids (1a), (1b), (1c) or (1d), in finely divided form, especially in droplet form, to the unladen microparticles. Surprisingly, the liquid droplets are absorbed rapidly by the unladen microparticles. In addition, it is possible in this way to exactly dose the liquid used for impregnation and hence the active, such that separation of excess liquid can be avoided, or the cost and inconvenience associated therewith reduced.

Therefore, the present invention also relates to a process for producing microparticles laden with at least one organic active of low molecular weight, wherein the microparticles have been formed from an organic, polymeric wall material and in the unladen state, in their interior, have at least one cavity connected via pores to the surface of the microparticles, wherein the unladen microparticles are impregnated with a liquid comprising the active, especially the liquid (1d), by applying the liquid in finely divided form, i.e. in droplet form or in the form of a spray mist, to the unladen microparticles. This process is also referred to hereinafter as method (e).

The microparticles laden with the active that are obtained by method (e) can, if desired, be sealed by a prior art method, i.e. by heating the microparticles to a temperature above the melting temperature or, if the wall material does not have a melting point, above the glass transition temperature. The microparticles laden with the active that are obtained by method e) can, if desired, also be sealed by means of one of the measures (a), (b), (c) described herein or especially by measure (d), by applying a substance that seals the pores of the laden microparticles to the surface of the laden microparticles.

The invention is associated with a number of advantages, some of which or especially all of which are achieved.
The microparticles that are used as starting material are easily and inexpensively producible.
The process for loading is very versatile with regard to the feedstocks to be used and is especially suitable for a multitude of actives of low molecular weight.
No thermal treatment of the laden microparticles is necessary for sealing of the pores since measures (a), (b), (c) and (d) achieve effective enclosure of the active of low molecular weight in the microparticles.
The microparticles laden with the active can be stored over a prolonged period without any significant loss of active.
The release characteristics of the active can be controlled via the choice of the respective substance A, B, C or D.
By the choice of the wall material and optionally of the substances A, B, C and D, the microparticles can be formed such that they are biodegradable.
The release of the active can be controlled in a simple manner.
If the active is an aroma chemical or a mixture of aroma chemicals, the aroma profile is generally maintained.

The present invention relates more particularly to the following items 1 to 50:
1. A process for producing microparticles laden with at least one organic active of low molecular weight, wherein the microparticles have been formed from an organic, polymeric wall material and in the unladen state, in their interior, have at least one cavity connected via pores to the surface of the microparticles, by one of methods A, B and C.
2. A process for producing microparticles laden with at least one organic active of low molecular weight, wherein the microparticles have been formed from an organic, polymeric wall material and in the unladen state, in their interior, have at least one cavity connected via pores to the surface of the microparticles, by method D.
3. A process for producing microparticles laden with at least one organic active of low molecular weight, wherein the microparticles have been formed from an organic, polymeric wall material and in the unladen state, in their interior, have at least one cavity connected via pores to the surface of the microparticles, wherein the unladen microparticles are impregnated with a liquid comprising the active by applying the liquid in finely divided form, especially droplet form, to the unladen microparticles.
4. The process according to item 1, wherein the nonpolymerizable substance A is selected from organic polymers that melt at a temperature in the range from 30 to 150° C., organic polymers that are solubilizable in any solvent present, and waxes, and mixtures thereof.
5. The process according to item 1, wherein the liquid (1a) used is a melt or solution consisting essentially of at least one active and at least one organic polymer and/or at least one wax, wherein the wax or organic polymer is in molten form or in the form of a solution in the active in the liquid.
6. The process according to either of items 4 and 5, wherein the nonpolymerizable substance A is selected from vegetable or animal waxes, polyalkylene glycols and mixtures thereof.
7. The process according to item 4, wherein the nonpolymerizable substance A is selected from water-solubilizable polymers.
8. The process according to item 7, wherein the liquid (1a) used is a mixture of an aqueous solution or emulsion of the water-solubilizable polymer and the active.
9. The process according to any of items 1 and 4 to 8, wherein the mass ratio of the at least one active to the nonpolymerizable substance A in the liquid (1a) is in the range from 99:1 to 10:90, especially in the range from 95:5 to 20:80.
10. The process according to item 1, wherein the polymerizable substance B is selected from ethylenically unsaturated monomers, silanes having hydroxyl or alkoxy groups, and oxidatively polymerizable aromatic compounds.
11. The process according to either of items 1 and 10, wherein the liquid (1b) used is an emulsion or solution consisting essentially of at least one active and at least one polymerizable substance B, wherein the polymerizable substance B is in molten form or in the form of a solution in the active in the liquid.
12. The process according to any of items 1, 10 and 11, wherein the mass ratio of the at least one active to the polymerizable substance B in the liquid (1b) is in the range from 99:1 to 10:90, especially in the range from 95:5 to 20:80.
13. The process according to item 2, wherein the liquid (1d) consists essentially of at least one liquid active.
14. The process according to either of items 2 and 13, wherein a solid coating is produced on the surface of the microparticles.
15. The process according to item 14, wherein the microparticles are treated with a liquid (2d) comprising
   i) at least one film-forming substance D in molten, emulsified, dispersed or dissolved form in the liquid, and
   ii) optionally one or more solvents,
   in such a way as to form a solid coating on the surface of the microparticles.
16. The process according to item 15, wherein the film-forming substance D is selected from organic polymers that melt at a temperature in the range from 30 to 150° C., organic polymers that are solubilizable and/or dispersible in any solvent present in the liquid (2d), and waxes.
17. The process according to item 16, wherein the liquid (2d) used is a melt or solution consisting essentially of at least one organic polymer and/or at least one wax, wherein the wax or organic polymer is in molten form or in the form of a solution, dispersion or emulsion in the solvent in the liquid.
18. The process according to item 16 or 17, wherein the film-forming substance D is selected from vegetable or animal waxes, polyalkylene glycols, homo- and copolymers of vinyl acetate and mixtures thereof.
19. The process according to item 16, wherein the film-forming substance D is selected from water-solubilizable and/or water-dispersible polymers.
20. The process according to item 19, wherein the liquid (2d) used is a solution, dispersion or emulsion of the water-solubilizable and/or water-dispersible polymer.
21. The process according to item 15, wherein the film-forming substance D is selected from a polymerizable substance and the film formation comprises a polymerization of substance D.
22. The process according to item 21, wherein the polymerizable substance is selected from ethylenically unsaturated monomers, silanes having hydroxyl or alkoxy groups, and oxidatively polymerizable aromatic compounds.
23. The process according to item 14, wherein a coating is produced on the surface of the microparticles by powdering the microparticles with a finely divided solid and then bringing about film formation on the surface of the microparticles.
24. The process according to item 14, wherein a coating is produced on the surface of the microparticles by depositing a volatile substance from the gas phase on the surface of the microparticles and converting it to a solid from the surface by chemical reaction.
25. The process according to any of the preceding items, wherein the treating of the microparticles with the liquid (1a), (1b), (1c) or (1d) is accomplished by using the microparticles in the form of a powder.
26. The process according to any of the preceding items, wherein the impregnating of the microparticles with the liquid (1a), (1b), (1c) or (1d) is accomplished by spray application or dropwise application of the respective liquid onto the microparticles or suspension of the microparticles in the respective liquid.
27. The process according to any of items 14 to 26, wherein the liquid (2d) is used in such an amount that the mass ratio of the microparticles obtained in step (d1) to substance D present in the liquid (2d) is in the range from 95:5 to 20:80.
28. The process according to any of items 2 and 14 to 27, wherein step (d2) is conducted in such a way that the thickness of the coating obtained averages in the range from 0.01 to 1.5 times the average radius of the microparticles.
29. The process according to any of the preceding items, wherein the impregnating of the microparticles with the liquid (1a), (1b), (1c) or (1d) is accomplished using a composition of microparticles in which the microparticles, prior to the filling, have an average particle diameter of 10 to 600 μm, wherein at least 80% of those microparticles having a particle diameter that differs from the average particle diameter of the microparticles in the composition by not more than 20% each have an average of at least 10 pores having a diameter in the range from 1/5000 to ⅕ of the average particle diameter, and, in addition, the diameter of each of these pores is at least 20 nm.
30. The process according to any of the preceding items, wherein the wall material comprises at least one polymer having a glass transition temperature or melting point in the range from 45 to 140° C.
31. The process according to any of the preceding items, wherein the wall material has a solubility in dichloromethane of at least 50 g/L at 25° C.
32. The process according to any of the preceding items, wherein the polymeric wall material comprises at least one aliphatic-aromatic polyester.
33. The process according to item 32, wherein the aliphatic-aromatic polyester is an ester of an aliphatic dihydroxyl compound esterified with a composition of aromatic dicarboxylic acid and aliphatic dicarboxylic acid.
34. The process according to item 33, wherein the aliphatic-aromatic polyester is selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST).
35. The process according to any of items 32 to 34, wherein the wall material, besides the aliphatic-aromatic polyester, additionally comprises at least one further polymer that is different from aliphatic-aromatic polyesters and that is especially selected from aliphatic polyesters, polyanhydrides, polyesteramides, modified polysaccharides and proteins.
36. The process according to item 35, wherein the further polymer is selected from polymerized hydroxycarboxylic acids, aliphatic-aliphatic polyesters, polylactones, poly(p-dioxanones), polyanhydrides and polyesteramides.
37. The process according to item 35, wherein the further polymer is selected from polylactic acid and aliphatic poly-C₅-C₁₂-lactones.
38. The process according to item 35, wherein the further polymer is selected from aliphatic-aliphatic polyesters and polyhydroxy fatty acids.
39. The process according to any of items 35 to 38, wherein the mass ratio of the aliphatic-aromatic polyester to the at least one further polymer that is different from aliphatic-aromatic polyesters is in the range from 30:70 to 99:1 or in the range from 30:70 to 80:20, in particular in the range from 35:65 to 75:25 and especially in the range from 40:60 to 70:30 or in the range from 30:70 to 70:30 and especially in the range from 45:55 to 70:30.
40. The process according to any of the preceding items, wherein the active is liquid at 22° C. and 1013 mbar or has a melting point below 100° C.
41. The process according to any of the preceding items, wherein the active is selected from aroma chemicals, organic crop protecting agents, organic pharmaceutical agents, cosmetic actives, and actives for construction chemical applications.
42. The process according to item 41, wherein the active is an aroma chemical which is liquid at 22° C. and 1013 mbar, or a mixture of aroma chemicals which is liquid at 22° C. and 1013 mbar.
43. The process according to item 42, wherein the aroma chemical comprises at least one volatile fragrance.
44. A composition of microparticles filled with at least one active, obtainable by a process according to any of the preceding items.
45. The composition according to item 44, comprising the active in a total amount of 5% to 75% by weight, based on the total weight of the laden microparticles.
46. The composition according to either of items 44 and 45 in the form of a powder.
47. A product comprising a composition according to any of items 44 to 46 in a proportion by weight of 0.01% to 80% by weight based on the total weight of the product.
48. The product according to item 47, wherein the product is selected from perfumes, washing products, cleaning products, cosmetic products, personal care products, hygiene articles, foods, food supplements, fragrance dispensers and fragrances.
49. The use of the composition according to any of items 44 to 46 in a product selected from perfumes, washing and cleaning products, cosmetic products, personal care products, hygiene articles, foods, food supplements, fragrance dispensers and fragrances.
50. The use of the composition according to any of items 44 to 46 for controlled release of actives.

The term “consisting essentially of” in relation to the liquids (1a), (1b), (1c) and (1d) is understood such that the sum total of the constituents is more than 80% by weight, further preferably at least 90% by weight, further preferably at least 95% by weight, further preferably at least 98% by weight, further preferably at least 99% by weight, further preferably at least 99.5% by weight, further preferably at least 99.9% by weight, based on the total weight of the liquid.

An active is understood by the person skilled in the art to mean chemical compounds that trigger a physiological effect in living beings and plants, and substances that cause a chemical effect or catalyze a chemical reaction in inanimate nature. Examples of actives are aroma chemicals, organic crop protecting agents, organic pharmaceutical agents, cosmetic actives and actives for uses in the construction sector, called construction chemicals, especially catalysts for products in the construction sector, e.g. crosslinking or polymerization catalysts.

The term “organic active of low molecular weight” refers to organic chemical compounds having a defined molecular weight Mn which is generally below 1000 daltons and typically in the range from 80 to <1000 daltons and especially in the range from 100 to 500 daltons.

The “molecular weight Mn” or the “molar mass Mn” is the number-average molecular weight or molar mass. The “molecular weight Mw” or the “molar mass Mw” is the mass-average molecular weight or molar mass. “Polydispersity” is the ratio of weight-average to number-average, i.e. the quotient Mw/Mn.

Unless stated otherwise, the term “room temperature” indicates a temperature of 22° C.

The term “biodegradable” is understood to mean that the substance in question, the unfilled microparticles here, in the test of OECD Guideline 301B from 1992 (measurement of evolution of CO₂ on composting in a mineral slurry and comparison with the theoretical maximum possible evolution of CO₂) after 28 days and 25° C. undergoes biodegradation of at least 5%, particularly at least 10% and especially at least 20%.

According to the invention, the unladen microparticles intended for impregnation are formed from an organic polymer material and have openings, called pores, on the particle surface. These pores are connected to one or more cavities in the interior of the microparticles, such that the respective liquids (1a), (1b), (1c) and (1d) can penetrate into the cavity through the pores on impregnation of the microparticles. In this way, the microparticles are laden with the active present in the liquids. In other words, in the impregnation, the microparticles are treated with the respective liquid (1a), (1b), (1c) or (1d) in such a way that the cavity present in the unfilled microparticles is largely or completely filled with the respective liquid and consequently laden with the active. The walls of these cavities are formed by the organic polymer material. In other words, the organic polymer material surrounds the cavities that are present in the microparticles and are connected by the pores, and is therefore also referred to as polymeric wall material. In the unfilled state, these cavities comprise a gas or gas mixture, typically air, CO₂ or an inert gas such as nitrogen or argon, which is largely or completely displaced on impregnation of the unladen microparticles with the respective liquid (1a), (1b), (1c) or (1d). The microparticles have one or more cavities in their interior. In the case of multiple cavities, the cavities may be separated from one another by the polymeric wall material or connected to one another. More particularly, the microparticles intended for loading have, in their interior, a multitude of mutually connected cavities, i.e. a network of cavities, connected by the pores in the surface of the microparticles.

The term “microparticles” means that the particles have dimensions in the micrometer range, i.e. below 1000 μm, particularly below 800 μm and especially below 600 μm.

The value reported here is that value that exceeds 90% by volume of the particles present in a sample, which is also referred to as the D[v, 09] value. Typically, at least 90% by volume of the microparticles intended for loading have dimensions of at least 1 μm, particularly at least 2 μm and especially at least 5 μm (called the D[v, 01] value).

The microparticles intended for impregnation or loading preferably have an average particle diameter, i.e. a D[4,3] value, of 1 to 600 μm, particularly of 5 to 500 μm and especially of 10 to 400 μm. In a first preferred embodiment, the average particle diameter D[4,3] is 1 to <100 μm, particularly 2 to 50 μm, especially 5 to 30 μm. In a second preferred embodiment, the average particle diameter D[4,3] is 30 to 600 μm, particularly 50 to 500 μm and especially 100 to 400 μm.

The microparticles intended for loading preferably have a Sauter diameter, i.e. a D[3,2] value, of 0.2 to 400 μm, particularly of 2.5 to 250 μm and especially of 5 to 200 μm. In a first preferred embodiment, the average particle diameter D[4,3] is 1 to <100 μm, particularly 2 to 50 μm, especially 5 to 30 μm. In a second preferred embodiment, the average particle diameter D[4,3] is 30 to 600 μm, particularly 50 to 500 μm and especially 100 to 400 μm.

The microparticles intended for impregnating preferably have a D[v, 05] value of 0.5 to 500 μm, particularly of 4 to 300 μm and especially of 10 to 300 μm. In a first preferred embodiment, the average particle diameter D[4,3] is 1 to <100 μm, particularly 2 to 50 μm, especially 5 to 30 μm. In a second preferred embodiment, the average particle diameter D[4,3] is 30 to 600 μm, particularly 50 to 500 μm and especially 100 to 400 μm.

Here and hereinafter, all figures for particle sizes, particle diameters and particle size distributions, including the D[v, 01], D[v, 05], D[v, 09], D[4,3] and D[3,2] values, are based on the particle size distributions ascertained by static laser light scattering to ISO 13320:2009 on samples of the microparticles. The abbreviation SLS is also used hereinafter for the expression “static laser light scattering to ISO 13320:2009”. In this connection, the D[v, 01] value means that 10% by volume of the particles of the measured sample have a particle diameter below the value reported as D[v, 01], Accordingly, the D[v, 05] value means that 50% by volume of the particles of the measured sample have a particle diameter below the value reported as D[v, 05], and the D[v, 09] value means that 90% by volume of the particles of the measured sample have a particle diameter below the value reported as D[v, 09], The D[4, 3] value is the volume-weighted average determined by means of SLS, which is also referred to as the De Brouckere mean and corresponds to the mass average for the particles of the invention. The D[3, 2] value is the surface-weighted average determined by means of SLS, which is also referred to as the Sauter diameter.

The microparticles intended for loading are preferably regular-shaped particles, especially sphere-shaped particles. The term “regular-shaped” means that the surface of the particles, apart from the pores, does not have any great depressions in the wall material or elevations of wall material. The term “spherical” means that the particles have approximately the shape of a rotational ellipsoid and especially a spherical shape, where, in a particle in particular, the ratio of the longest axis through the center of the particle to the shortest axis through the center of the particle does not exceed a value of 2 and is especially in the range from 1:1 to 1.5:1.

The microparticles intended for loading are especially spherical microparticles that preferably have an average particle diameter D[4,3] of 1 to 600 μm, particularly of 5 to 500 μm and especially of 10 to 400 μm. In a first preferred embodiment, the average particle diameter D[4,3] of the spherical microparticles is 1 to <100 μm, particularly 2 to 50 μm, especially 5 to 30 μm. In a second preferred embodiment, the average particle diameter D[4,3] is 30 to 600 μm, particularly 50 to 500 μm and especially 100 to 400 μm.

Preferably, the microparticles intended for loading have at least 10, preferably at least 20, pores on their surface. Preferably, the diameter of the pores is in the range from 1/5000 to ⅕ of the average particle diameter. The diameter of these pores is preferably at least 20 nm, particularly at least 50 nm, more preferably at least 100 nm and especially at least 200 nm. The diameter of these pores will generally not exceed 20 μm, especially 10 μm, and is particularly in the range from 100 nm to 20 μm and especially 200 nm to 10 μm, depending on the respective average particle diameter D[4,3].

Especially preferred are microparticles intended for loading that have an average particle diameter D[4,3] in the range from 10 to 600 μm, particularly of 30 to 500 μm and especially of 50 to 400 μm, where at least 80% of those microparticles that have a particle diameter that differs from the average particle diameter of the microparticles in the composition by not more than 20% each have an average of at least 10 pores having a diameter in the range from 1/5000 to ⅕ of the average particle diameter, and, in addition, the diameter of each of these pores is at least 20 nm, especially at least 50 nm, more preferably at least 100 nm and especially at least 200 nm. The diameter of these pores will generally not exceed 20 μm, especially 10 μm, and is particularly in the range from 100 nm to 20 μm and especially 200 nm to 10 μm.

The pore diameters specified here can be determined by means of scanning electron microscopy (Phenom Pro X) by the method that follows. For this purpose, various close-ups are taken and afterwards these are automatically analyzed with the ProSuite (FibreMetric) software from Phenom. The pores of a selected region of a particle are recognized by the difference in contrast and the areas thereof are measured automatically. Assuming that the surfaces are circular, the diameter is calculated for each surface (sample size: 100 pores).

The evaluation only takes account of those pores having a pore diameter of at least 20 nm. According to the particle size, the images are made, in the case of larger particles, with 1600- to 2400-fold magnification and, in the case of smaller particles, with up to 8000-fold magnification.

In order to determine the size of at least 10 pores, microparticles considered are those having a particle diameter that differs by not more than 20% from the average particle diameter of the composition of the microparticles.

For the evaluation of the number of pores based on the total surface area of the microparticle, the following assumptions are made: since the particle will generally be spherical, the image shows only half of the surface of the particle. If the image of a microparticle shows at least 5 pores having a diameter of at least 20 nm and the diameter is in the range from 1/5000 to ⅕ of the average particle diameter, the overall surface will comprise at least 10 pores.

The evaluation of the data thus obtained is performed as follows:
1. The average particle diameter D[4,3] of the microparticles is determined directly on the microparticle dispersion by means of light scattering. Purely theoretically, this gives the upper limit and lower limit of the particle diameter of the microparticles that are taken into account for the determination of the pores (±20%).
2. The microparticle dispersion is dried.
3. In each case 20 images of a sample showing multiple microparticles are taken by scanning electron microscopy.
4. 20 microparticles having particle diameters in the range of ±20% of the average particle diameter of the microparticles are selected. The particle diameter thereof is measured with the ProSuite (FibreMetric) software from Phenom.
5. The pores of each of these 20 microparticles are measured. For this purpose, the surface areas of the visible pores are measured automatically and their diameter is calculated.
6. The individual values of the pore diameters are checked as to whether their diameter satisfies the condition of being within the range from 1/5000 to ⅕ of the average particle diameter and is at least 20 nm.
7. The number of pores that meet this condition is determined and multiplied by two.
8. It is checked whether at least 16 microparticles have an average of in each case at least 10 pores.

According to the invention, the microparticles are formed from an organic polymeric wall material. The polymeric wall material may in principle be any organic polymers as used in a known manner for production of porous, gas-filled microparticles. Examples of such polymeric wall materials are in particular condensation polymers such as polyesters, including aliphatic polyesters, semiaromatic polyesters and aromatic polyesters, and also polyamides, polyesteramides, polycarbonates, but also addition polymers, such as polystyrenes, polyacrylates, polyolefins, polyureas and polyurethanes, including polyesterurethanes and polyetherurethanes, and blends of the aforementioned polymers. Preferably, the wall material comprises at least one condensation polymer, especially at least one polyester.

Preferably, the wall material comprises at least one polymer having a glass transition temperature or melting point in the range from 45 to 140° C. If the polymer has a melting point, i.e. is semicrystalline or crystalline, it preferably has a melting point in the range from 45 to 140° C. If the polymer is amorphous, it preferably has a glass transition temperature in the range from 45 to 140° C. The glass transition temperature here is typically determined by means of dynamic differential calorimetry (DSC) to DIN EN ISO 11357.

Preferably, the wall material has a solubility in dichloromethane of at least 50 g/L at 25° C.

The wall material especially comprises at least one semiaromatic polyester as main constituent. Semiaromatic polyesters are also referred to as aliphatic-aromatic polyesters, i.e. polyesters based on aromatic dicarboxylic acids and aliphatic dihydroxyl compounds, and polyesters based on mixtures of aromatic dicarboxylic acids with aliphatic dicarboxylic acids and aliphatic dihydroxyl compounds. Aliphatic-aromatic polyesters are preferably polyesters based on mixtures of aliphatic dicarboxylic acids with aromatic dicarboxylic acids and aliphatic dihydroxyl compounds. These polymers may be present individually or in the mixtures thereof. Wall materials based on semiaromatic polyesters of this kind are typically biodegradable for the purposes of this invention, and hence so are the unfilled microparticles produced therefrom.

Preferably, “aliphatic-aromatic polyesters” shall also be understood to mean polyester derivatives such as polyetheresters, polyesteramides or polyetheresteramides and polyesterurethanes, as described, for example, in WO 2012/2013506. The suitable aliphatic-aromatic polyesters include linear, non-chain-extended polyesters, as described for example in WO 92/09654. Preference is given to chain-extended and/or branched aliphatic-aromatic polyesters. The latter are known from WO 96/15173, WO 96/15174, WO 96/15175, WO 96/15176, WO 96/21689, WO 96/21690, WO 96/21691, WO 96/21692, WO 96/25446, WO 96/25448 and WO 98/12242, to which explicit reference is made. Likewise considered are mixtures of different aliphatic-aromatic polyesters. Interesting recent developments are based on renewable raw materials and are described inter alia in WO 2006/097353, WO 2006/097354 and WO 2010/034710.

Particularly preferred aliphatic-aromatic polyesters include polyesters comprising as essential components:

A) an acid component formed from
a1) 30 to 99 mol % of at least one aliphatic dicarboxylic acid or the ester-forming derivatives thereof or mixtures thereof,
a2) 1 to 70 mol % of at least one aromatic dicarboxylic acid or the ester-forming derivative thereof or mixtures thereof and
   B) at least one diol component selected from C₂- to C₁₂-alkanediols and
   C) optionally a component selected from
c1) a compound having at least three groups capable of ester formation,
c2) a diisocyanate or polyisocyanate,
c3) a diepoxide or polyepoxide.

Aliphatic dicarboxylic acids and the ester-forming derivatives thereof (a1) that are generally considered are aliphatic dicarboxylic acids having 2 to 18 carbon atoms, preferably 4 to 10 carbon atoms, and the ester-forming derivatives thereof. They may be either linear or branched. However, it is also possible in principle to employ dicarboxylic acids having a greater number of carbon atoms, for example having up to 30 carbon atoms.

Examples of aliphatic dicarboxylic acids and the ester-forming derivatives thereof (a1) include: oxalic acid, malonic acid, succinic acid, 2-methylsuccinic acid, glutaric acid, 2-methylglutaric acid, 3-methylglutaric acid, α-ketoglutaric acid, adipic acid, pimelic acid, azelaic acid, sebacic acid, brassylic acid, fumaric acid, 2,2-dimethylglutaric acid, suberic acid, diglycolic acid, oxaloacetic acid, glutamic acid, aspartic acid, itaconic acid and maleic acid. These dicarboxylic acids or the ester-forming derivatives thereof may be used individually or as a mixture of two or more thereof.

It is preferable to employ succinic acid, adipic acid, azelaic acid, sebacic acid, brassylic acid or their respective ester-forming derivatives or mixtures thereof as component (a1). It is particularly preferable to employ succinic acid, adipic acid or sebacic acid or the respective ester-forming derivatives thereof or mixtures thereof. Succinic acid, azelaic acid, sebacic acid and brassylic acid additionally have the advantage that they are obtainable from renewable raw materials.

More preferably, the aliphatic-aromatic polyester is selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST).

The aromatic dicarboxylic acids or the ester-forming derivatives thereof (a2) may be used individually or as a mixture of two or more thereof. Particular preference is given to using terephthalic acid or the ester-forming derivatives thereof such as dimethyl terephthalate.

Generally, the diols (B) are selected from branched or linear alkanediols having 2 to 12 carbon atoms, preferably 4 to 6 carbon atoms, or cycloalkanediols having 5 to 10 carbon atoms. Examples of suitable alkanediols are ethylene glycol, propane-1,2-diol, propane-1,3-diol, butane-1,2-diol, butane-1,4-diol, pentane-1,5-diol, 2,4-dimethyl-2-ethylhexane-1,3-diol, 2,2-dimethylpropane-1,3-diol, 2-ethyl-2-butylpropane-1,3-diol, 2-ethyl-2-isobutylpropane-1,3-diol, 2,2,4-trimethylhexane-1,6-diol, especially ethylene glycol, propane-1,3-diol, butane-1,4-diol and 2,2-dimethylpropane-1,3-diol (neopentyl glycol). Examples of cycloalkanediols are cyclopentanediol, cyclohexane-1,4-diol, cyclohexane-1,2-dimethanol, cyclohexane-1,3-dimethanol, cyclohexane-1,4-dimethanol and 2,2,4,4-tetramethylcyclobutane-1,3-diol. The aliphatic-aromatic polyesters may also comprise mixtures of different alkanediols in condensed form. Particular preference is given to butane-1,4-diol, especially in combination with adipic acid as component a1), and propane-1,3-diol, especially in combination with sebacic acid as component a1). Propane-1,3-diol also has the advantage that it is obtainable as a renewable raw material.

The preferred aliphatic-aromatic polyesters are characterized by a molecular weight Mn in the range from 1000 to 100 000 g/mol, especially in the range from 9000 to 75 000 g/mol, preferably in the range from 10 000 to 50 000 g/mol.

Preferably, at least one of the polymers contained in the continuous phase of a) has a glass transition temperature or a melting point in the range from 45 to 140° C.

In a preferred embodiment, the wall material of the microparticles consists essentially, preferably to an extent of at least 95% by weight, especially to an extent of at least 99% by weight, based on the wall material, of at least one aliphatic-aromatic polyester.

In a preferred embodiment, the wall material comprises, besides the aliphatic-aromatic polyester, additionally at least one further polymer that is not an aliphatic-aromatic polyester.

Examples of polymers that are not aliphatic-aromatic polyesters include: polyacrylates, polyamides, polycarbonates, polystyrenes, aliphatic polyesters, polyether esters, polyanhydrides, polyesteramides, furthermore aromatic/aromatic polyesters, polyolefins, polyureas, polyurethanes, modified polysaccharides and proteins.

This at least one further polymer is preferably selected from aliphatic polyesters, aliphatic polyanhydrides, aliphatic polyetheresters, aliphatic polyesteramides, modified polysaccharides and proteins and mixtures thereof, and is especially selected from polymerized hydroxycarboxylic acids, aliphatic-aliphatic polyesters, polylactones, poly(p-dioxanones), polyanhydrides and polyesteramides. The at least one further polymer is more preferably selected and aliphatic polyesters and especially from polylactic acid, aliphatic-aliphatic polyesters and poly-C₆-C₁₂-lactones.

The particularly preferred group of aliphatic polyesters includes polyhydroxy fatty acids including poly-C₆-C₁₂-lactones, polyhydroxyacetic acid, polylactic acid and aliphatic-aliphatic polyesters, and mixtures thereof.

In a preferred group of embodiments, the further polymer comprises an aliphatic polyester from the group which polyhydroxyacetic acids and polylactic acid and copolymers thereof. Among these, preference is given to polylactic acid or polylactide, which are also referred to as PLA, and PLA copolymers, i.e. polylactide or polylactic acid copolymers, for example PLGA, i.e. polylactide-co-glycolide. Among PLA and PLA copolymers, preference is given to polylactic acid. Polylactic acid having a molecular weight of 30 000 to 120 000 daltons and a glass transition temperature (Tg) in the range from 50 to 65° C. is particularly suitable. Most preferably, amorphous polylactic acid having a proportion of D-lactic acid greater than 9% is used.

In a further preferred group of embodiments, the further polymer comprises an aliphatic polyester from the group of the polyhydroxy fatty acids. Polyhydroxy fatty acids are understood to mean polyesters based on hydroxy fatty acid that bear an aliphatic hydrocarbyl radical typically having 1 to 18 carbon atoms, especially 1 to 6 carbon atoms, between the carbon atom that bears the OH group and the carbon atom of the carboxyl group. Polyhydroxy fatty acids are also understood to mean polyesters of 2-hydroxybutyric acid, especially homopolymers thereof. Polylactic acid and polyhydroxyacetic acid are accordingly not polyhydroxy fatty acids. Polyhydroxy fatty acids typically comprise repeat monomer units of the formula (1)

[—O—CHR—(CH₂)ₘ—CO—] (1)

where R is hydrogen or a linear or branched alkyl group having 1 to 20, preferably 1 to 16, carbon atoms, especially 1 to 6 carbon atoms, and m=numbers from 1 to 18, preferably 1, 2, 3, 4, 5 and 6; and/or polyesters of 2-hydroxybutyric acid. Preferred polyhydroxy fatty acids are those having repeat units of the formula (1).

The polyhydroxy fatty acids include homopolymers (synonym: homopolyesters), i.e. polyhydroxy fatty acids consisting of identical hydroxy fatty acid monomers, and copolymers (synonym: copolyesters), i.e. polyhydroxy fatty acids consisting of different hydroxy fatty acid monomers. The polyhydroxy fatty acids may be used individually or in any desired mixtures.

Polyhydroxy fatty acids especially have molecular weights M_{w} of 5000 to 1 000 000, especially of 30 000 to 1 000 000, especially of 70 000 to 1 000 000, preferably of 100 000 to 1 000 000 or of 300 000 to 600 000 and/or melting temperatures in the range from 100 to 190° C.

In one embodiment of the invention, the at least one polyhydroxy fatty acid is selected from the group consisting of
poly-3-hydroxypropionates (P3HP);
polyhydroxybutyrates (PHB);
polyhydroxyvalerates (PHV);
polyhydroxyhexanoates (PHHx);
polyhydroxyoctanoates (PHO);
polyhydroxyoctadecanoates (PHOD);
copolyesters of hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids;
copolyesters of hydroxyvaleric acid with at least one monomer selected from the group consisting of 3-hydroxypropionic acid, hydroxyhexanoic acids, hydroxyoctanoic acids and hydroxyoctadecanoic acids;
copolyesters of hydroxyhexanoic acid with at least one monomer selected from the group consisting of 3-hydroxypropionic acid, hydroxyoctanoic acid and hydroxyoctadecanoic acids and
poly-C₆-C₁₂-lactones, especially polycaprolactones.

Suitable polyhydroxybutyrates (PHB) may be selected from the group consisting of poly-2-hydroxybutyrates (P2HB), poly-3-hydroxybutyrates (P3HB), poly-4-hydroxybutyrates (P4HB) and copolymers of at least 2 hydroxy butyric acids selected from the group consisting of 2-hydroxybutyric acid, 3-hydroxy butyric acid and 4-hydroxybutyric acid. Also suitable are copolymers of 3-hydroxybutyric acid and 4-hydroxybutyric acid. These copolymers are characterized by the following brief notation: [P(3HB-co-4HB)] where 3HB stands for 3-hydroxybutyrate and 4HB for 4-hydroxybutyrates.

Poly-3-hydroxybutyrates are sold, for example, by PHB Industrial under the Biocycle® brand name and by Tianan under the Enmat® name. Poly-3-hydroxybutyrate-co-4-hydroxybutyrates are especially known from Metabolix. They are sold under the Mirel® brand name.

Suitable polyhydroxyvalerates (PHV) may be selected from the group consisting of homopolymers of 3-hydroxyvaleric acid [=poly-3-hydroxyvalerates (P3HV)], homopolymers of 4-hydroxyvaleric acid [=poly-4-hydroxyvalerates (P4HV)]; homopolymers of 5-hydroxyvaleric acid [=poly-5-hydroxyvalerates (P5HV)]; homopolymers of 3-hydroxymethylvaleric acid[=poly-3-hydroxymethylvalerates (P3MHV)]; copolymers of at least 2 hydroxyvaleric acids selected from the group consisting of 3-hydroxyvaleric acid, 4-hydroxyvaleric acid, 5-hydroxyvaleric acid and 3-hydroxymethylvaleric acid.

Suitable polyhydroxyhexanoates (PHHx) may be selected from the group consisting of poly-3-hydroxyhexanoates (P3HHx), poly-4-hydroxyhexanoates (P4HHx), poly-6-hydroxyhexanoates (P6HHx), and copolymers of at least 2 hydroxyhexanoic acids selected from the group consisting of 3-hydroxyhexanoic acid, 4-hydroxyhexanoic acid and 6-hydroxyhexanoic acid.

Suitable polyhydroxyoctanoates (PHO) may be selected from the group consisting of poly-3-hydroxyoctanoates (P3HO), poly-4-hydroxyoctanoates (P4HO), poly-6-hydroxyoctanoates (P6HO), and copolymers of at least 2 hydroxyoctanoic acids selected from the group consisting of 3-hydroxyoctanoic acid, 4-hydroxyoctanoic acid and 6-hydroxyoctanoic acid.

Suitable copolyesters of hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids are, for example: copolyesters of 4-hydroxybutyric acid with 3-hydroxyvaleric acid
[P(4HB-co-3HV)], copolyesters of 3-hydroxybutyric acid with 3-hydroxyvaleric acid
[P(3HB-co-3HV)], copolyesters of 4-hydroxybutyric acid with 3-hydroxyhexanoic acid
[P(4HB-co-3HHx)], copolyesters of 3-hydroxybutyric acid with 3-hydroxyhexanoic acid
[P(3HB-co-3HHx)], copolyesters of 4-hydroxybutyric acid with 3-hydroxyoctanoic acid
[P(4HB-co-3HO)], copolyesters of 3-hydroxybutyric acid with 3-hydroxyoctanoic acid [P(3HB-co-3HO)],
copolyesters of 4-hydroxybutyric acid with 3-hydroxyoctadecanoic acid [P(4HB-co-3HOD)] and copolyesters of 3-hydroxybutyric acid with 3-hydroxyoctadecanoic acid
[P(3HB-co-3HOD)].

Preference is given to using poly-3-hydroxybutyrate-co-3-hydroxyhexanoates having a 3-hydroxyhexanoate content of 1 to 20 and preferably of 3 to 15 mol %, based on the total amount of the polyhydroxy fatty acid. Such poly-3-hydroxybutyrate-co-3-hydroxyhexanoates [P(3HB-co-3HHx] are known from Kaneka and are commercially available under the Aonilex™ X131A and Aonilex™ X151A brand names.

Suitable copolyesters of hydroxyvaleric acid are preferably copolyesters of 4-hydroxyvaleric acid and/or 3-hydroxyvaleric acid with at least one monomer selected from the group consisting of 3-hydroxypropionic acid, hydroxyhexanoic acids, hydroxyoctanoic acids, especially 3-hydroxyoctanoic acid, and hydroxyoctadecanoic acids.

Suitable copolyesters of hydroxyhexanoic acid are preferably copolyesters of 3-hydroxyhexanoic acid with at least one monomer selected from the group consisting of 3-hydroxypropionic acid and hydroxyoctanoic acid, preferably 3-hydroxyoctanoic acid, and hydroxyoctadecanoic acids.

In a further preferred group of embodiments, the further polymer comprises an aliphatic polyester from the group of the polylactones, especially the poly-C₆-C₁₂-lactones, specifically the polycaprolactones. Polylactones refer to polyesters obtainable by ring-opening polymerization of lactones, especially of C₆-C₁₂-lactones and specifically epsilon-caprolactone (ε-caprolactone). Poly-C₆-C₁₂-lactones are accordingly polyhydroxy fatty acids having repeat monomer units of the general formula (1) [—O—CHR—(CH₂)ₘ—CO—] in which m is 4 to 10, m=4 in the case of caprolactone, and in which R is hydrogen. The term “polycaprolactone” in the context of the invention is understood to mean both homopolymers of epsilon-caprolactone and copolymers of epsilon-caprolactone. Suitable copolymers are, for example, copolymers of epsilon-caprolactone with monomers selected from the group consisting of lactic acid, lactide, hydroxyacetic acid and glycolide. Preferred polycaprolactones are polycaprolactone (PCL), polycaprolactone-co-lactide and polyglycolide-co-lactide-co-caprolactone.

Polycaprolactones are sold, for example, by Perstorp under the Capa™ brand name or by Daicel under the Celgreen™ brand name.

In a preferred group of embodiments, the further polymer of the wall material comprises a polycaprolactone.

In a further preferred group of embodiments of the invention, the further polymer of the wall material comprises at least one polyhydroxy fatty acid selected from the group consisting of
poly-3-hydroxypropionates (P3HP);
polyhydroxybutyrates (PHB);
polyhydroxyvalerates (PHV);
polyhydroxyhexanoates (PHHx);
polyhydroxyoctanoates (PHO);
polyhydroxyoctadecanoates (PHOD);
copolyesters of hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids;
copolyesters of hydroxyvaleric acid with at least one monomer selected from the group consisting of 3-hydroxypropionic acid, hydroxyhexanoic acids, hydroxyoctanoic acids and hydroxyoctadecanoic acids;
copolyesters of hydroxyhexanoic acid with at least one monomer selected from the group consisting of 3-hydroxypropionic acid, hydroxyoctanoic acid and hydroxyoctadecanoic acids and
polycaprolactones.

In a further preferred group of embodiments of the invention, the further polymer of the wall material comprises at least one polyhydroxy fatty acid selected from the group consisting of poly-3-hydroxypropionates (P3HP);poly-2-hydroxybutyrates (P2HB); copolymers of at least 2 hydroxybutyric acids selected from the group consisting of 2-hydroxybutyric acid, 3-hydroxybutyric acid and 4-hydroxybutyric acid; copolymers of 3-hydroxybutyric acid and 4-hydroxybutyric acid; poly-3-hydroxyvalerate (P3HV); poly-4-hydroxyvalerate (P4HV); poly-5-hydroxyvalerates (P5HV); poly-3-hydroxymethylvalerates (P3MHV); copolymers of at least 2 hydroxyvaleric acids selected from the group consisting of 3-hydroxyvaleric acid, 4-hydroxyvaleric acid, 5-hydroxyvaleric acid and 3-hydroxymethylvaleric acid; poly-3-hydroxyhexanoates (P3HHx); poly-4-hydroxyhexanoates (P4HHx); poly-6-hydroxyhexanoates (P6HHx); copolymers of at least 2 hydroxyhexanoic acids selected from the group consisting of 3-hydroxyhexanoic acid, 4-hydroxyhexanoic acid and 6-hydroxyhexanoic acid; poly-3-hydroxyoctanoate (P3HO); poly-4-hydroxyoctanoate (P4HO); poly-6-hydroxyoctanoate (P6HO); copolymers of at least 2 hydroxyoctanoic acids selected from the group consisting of 3-hydroxyoctanoic acid, 4-hydroxyoctanoic acid and 6-hydroxyoctanoic acid; poly-3-hydroxyoctanoate (P3HO); poly-4-hydroxyoctanoate (P4HO); poly-6-hydroxyoctanoate (P6HO); copolymers of at least 2 hydroxyoctanoic acids selected from the group consisting of 3-hydroxyoctanoic acid, 4-hydroxyoctanoic acid and 6-hydroxyoctanoic acid; copolyesters of 2-hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids; copolyesters of 4-hydroxybutyric acid with 3-hydroxyoctanoic acid [P(4HB-co-3HO)], copolyesters of 3-hydroxybutyric acid with 3-hydroxyoctanoic acid [P(3HB-co-3HO)], copolyesters of 4-hydroxybutyric acid with 3-hydroxyoctadecanoic acid [P(4HB-co-3HOD)], copolyesters of 3-hydroxybutyric acid with 3-hydroxyoctadecanoic acid [P(3HB-co-3HOD)]; copolyesters of hydroxyvaleric acid, especially of 3-hydroxyvaleric acid or 4-hydroxyvaleric acid with at least one monomer selected from the group consisting of 3-hydroxypropionic acid, hydroxyhexanoic acids, hydroxyoctanoic acids and hydroxyoctadecanoic acids; copolyesters of 3-hydroxyhexanoic acid with at least one monomer selected from the group consisting of 3-hydroxypropionic acid, hydroxyoctanoic acid, preferably 3-hydroxyoctanoic acid and hydroxyoctadecanoic acids; and polycaprolactones.

In a further preferred group of embodiments of the invention, the further polymer of the wall material comprises at least one polyhydroxy fatty acid selected from the group of the polyhydroxyalkanoates. Polyhydroxyalkanoates are understood to mean primarily poly-4-hydroxybutyrates and poly-3-hydroxybutyrates, and also include copolyesters of the aforementioned hydroxybutyrates with 3-hydroxyvalerates (P(3HB)-co-P(3HV)) or 3-hydroxyhexanoate. The polyhydroxyalkanoates generally have a molecular weight Mw of 30 000 to 1 000 000 g/mol and preferably of 100 000 to 600 000 g/mol.

In a further preferred group of embodiments of the invention, the further polymer of the wall material comprises at least one aliphatic-aliphatic polyester. Aliphatic-aliphatic polyesters are understood to mean polyesters based on aliphatic dicarboxylic acids and aliphatic dihydroxy compounds, and polyesters based on mixtures of aliphatic dicarboxylic acids with aliphatic dicarboxylic acids and aliphatic dihydroxy compounds.

Examples of aliphatic carboxylic acids suitable for preparation of the aliphatic-aliphatic polyesters are the aliphatic dicarboxylic acids mentioned under (a1), especially those having 2 to 18 carbon atoms, preferably 4 to 10 carbon atoms. Preference is given to aliphatic-aliphatic polyesters in which the aliphatic dicarboxylic acid is selected from succinic acid, adipic acid, azelaic acid, sebacic acid, brassylic acid and mixtures thereof. Particular preference is given to succinic acid, adipic acid and sebacic acid, and mixtures thereof. For preparation of the aliphatic-aliphatic polyesters, rather than the dicarboxylic acids, it is also possible to use their respective ester-forming derivatives or mixtures thereof with the aliphatic dicarboxylic acids.

Examples of aliphatic diols suitable for preparation of the aliphatic-aliphatic polyesters are the diols mentioned as component (B), for example branched or linear alkanediols having 2 to 12 carbon atoms, preferably 4 to 6 carbon atoms, or cycloalkanediols having 5 to 10 carbon atoms. Examples of suitable alkanediols are especially ethylene glycol, propane-1,2-diol, propane-1,3-diol, butane-1,2-diol, butane-1,4-diol, pentane-1,5-diol, 2,4-dimethyl-2-ethylhexane-1,3-diol, 2,2-dimethylpropane-1,3-diol, 2-ethyl-2-butylpropane-1,3-diol, 2-ethyl-2-isobutylpropane-1,3-diol, 2,2,4-trimethylhexane-1,6-diol, especially ethylene glycol, propane-1,3-diol, butane-1,4-diol and 2,2-dimethylpropane-1,3-diol (neopentyl glycol). Examples of cycloalkanediols are cyclopentanediol, cyclohexane-1,4-diol, cyclohexane-1,2-dimethanol, cyclohexane-1,3-dimethanol, cyclohexane-1,4-dimethanol and 2,2,4,4-tetramethylcyclobutane-1,3-diol. The aliphatic polyesters may also comprise mixtures of different alkanediols in copolymerized form. Particular preference is given to butane-1,4-diol, especially in combination with one or two aliphatic dicarboxylic acids selected from succinic acid, adipic acid and sebacic acid as component a1).

Examples of particularly preferred aliphatic-aliphatic polyesters are polybutylene succinate adipate, polybutylene succinate, polybutylene sebacate, polybutylene succinate sebacate.

The preferred aliphatic-aliphatic polyesters frequently have a molecular weight Mn in the range from 1000 to 100 000 g/mol, particularly in the range from 9000 to 2000 g/mol, especially in the range from 5000 to 50 000 g/mol.

In a further preferred group of embodiments of the invention, the further polymer of the wall material comprises a poly-p-dioxanone (poly-1,4-dioxan-2-one). Poly-p-dioxanone refers to poly(etheresters) obtainable by ring-opening polymerization from 1,4-dioxan-2-one. The term “poly(p-dioxanones)” in the context of the present invention is understood to mean homopolymers of 1,4-dioxan-2-one that have the general structural unit [—O—CH₂—CH₂—O—CH₂—CO—]ₙ. The term “poly(p-dioxanones)” in the context of the present invention is also understood to mean copolymers of 1,4-dioxan-2-one with lactone monomers. Copolymers of 1,4-dioxan-2-one with at least one further monomer selected from the group consisting of glycolide, lactide and epsilon-caprolactone are especially suitable.

In a further preferred group of embodiments of the invention, the further polymer of the wall material comprises a polyanhydride. Polyanhydrides refer to polymers having the general structural unit

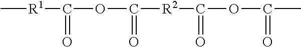

as characteristic base units of the main chain. R¹ and R² may be identical or different aliphatic or aromatic radicals. Suitable polyanhydrides are described in Kumar et al, Adv. Drug Delivery Reviews 54 (2002), p. 889-910. Especially suitable polyanhydrides are those described in Kumar et al. Adv. Drug Delivery Reviews 54 (2002), on page 897, to which reference is made here in full. In one embodiment of the invention, the polyanhydride is selected from the group of the aliphatic polyanhydrides, especially from the group consisting of polysebacic acid and polyadipic acid.

Suitable further polymers that may be used as wall material in combination with the aliphatic-aromatic polyester are polyesteramides, especially aliphatic polyesteramides. Aliphatic polyesteramides are copolymers of aliphatic polyamides and aliphatic polyesters, and hence polymers that bear both amide and ester functions. Suitable polyesteramides are especially polyesteramides that are obtained by condensation of ε-caprolactam, adipic acid and butane-1,4-diol, and polyesteramides that are obtained by condensation of adipic acid, butane-1,4-diol, diethylene glycol and hexamethylenediamine. Polyesteramides are sold, for example, under the BAK™ trade name by Bayer, for example BAK™1095 or BAK™ 2195.

Suitable further polymers that may be used as wall material in combination with the aliphatic-aromatic polyester are polysaccharides. Polysaccharides are macromolecules in which a relatively large number of sugar residues are glycosidically linked to one another. Polysaccharides suitable in accordance with the invention are especially polysaccharides having a solubility at 25° C. of at least 50 g/l in dichloromethane. Polysaccharides in the context of the invention also include derivatives thereof, provided that they have a solubility at 25° C. of at least 50 g/l in dichloromethane.

Polysaccharides suitable in accordance with the invention are preferably selected from the group consisting of modified starches such as, in particular, starch ethers and esters, cellulose derivatives such as, in particular, cellulose esters and cellulose ethers, chitin derivatives, chitosan derivatives.

Cellulose derivatives generally refer to celluloses chemically modified by polymer-analogous reactions. They include both products in which exclusively the hydroxy hydrogen atoms of the glucose units of the cellulose are replaced by organic or inorganic groups and those in which there has been a formal exchange of the entire hydroxyl groups (e.g. deoxycelluloses). Products that are obtained with intramolecular elimination of water (anhydrocelluloses), oxidation reactions (aldehyde, ketone and carboxy celluloses) or cleavage of the C₂, C₃-carbon bond of the glucose units (dialdehyde and dicarboxy celluloses) are also counted among the cellulose derivatives. Cellulose derivatives are finally also obtainable by reactions such as crosslinking or graft copolymerization reactions. Since a multitude of reagents is used to some degree for all these reactions and the degrees of substitution and polymerization of the cellulose derivatives obtained can additionally be varied, an extensive range of soluble and insoluble cellulose derivatives with widely varying properties is known.

Suitable cellulose ethers are, for example, methyl cellulose, ethyl cellulose, hydroxyethyl cellulose, hydroxypropyl cellulose and hydroxypropyl methylcellulose.

Suitable cellulose ethers are methyl hydroxy-(C₁-C₄)-alkyl celluloses. Methyl hydroxy-(C₁-C₄)-alkyl celluloses are methyl hydroxy-(C₁-C₄)-alkyl celluloses of a wide variety of different degrees of methylation and degrees of alkoxylation.

The preferred methyl hydroxy-(C₁-C₄)-alkyl celluloses have an average degree of substitution DS of 1.1 to 2.5 and a molar degree of substitution MS of 0.03 to 0.9. Suitable methyl hydroxy-(C₁-C₄)-alkyl celluloses are, for example, methyl hydroxyethyl cellulose or methyl hydroxypropyl cellulose.

Suitable cellulose esters are, for example, the esters of cellulose with C₂-C₄ monocarboxylic acids, such as cellulose acetate (commercially available from Eastmann CA-398-3), cellulose butyrates, cellulose acetobutyrates, cellulose propionate and cellulose acetopropionate. Cellulose esters are available in a wide variety of different degrees of polymerization and substitution.

Suitable further polymers that may be used as wall material in combination with the aliphatic-aromatic polyester are proteins. Proteins to be used in accordance with the invention include polypeptides (condensation products of amino acids having an acid amide-type linkage by a peptide bond) and derivatives thereof that have a solubility at 25° C. of at least 50 g/l in dichloromethane. The polypeptides may be of natural or synthetic origin.

In particularly preferred embodiments, the wall material comprises or consists of a combination of
i) at least one aliphatic-aromatic polyester selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
ii) at least one aliphatic polyester selected from aliphatic polyesters, especially from polyhydroxy fatty acids including poly-C₆-C₁₂-lactones, polyhydroxyacetic acid, polylactic acid and aliphatic-aliphatic polyesters and mixtures thereof, and specifically from polylactic acid, aliphatic-aliphatic polyesters and poly-C₆-C₁₂-lactones.

In specific embodiments, the wall material comprises or consists of a combination of
i) at least one aliphatic-aromatic polyester selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
ii) at least one aliphatic polyester selected from polycaprolactones, polylactic acid (PLA), polylactide glycolide, polybutylene succinate adipate, polybutylene succinate, polybutylene sebacate, polybutylene succinate sebacate.

Preference is given in accordance with the invention to mixtures of at least one aliphatic-aromatic polyester with one or more polymers that are not aliphatic-aromatic polyesters, with a proportion by weight of the aromatic-aliphatic polyester of 30% to 99% by weight, based on the total weight of aliphatic-aromatic polyester and the polymer that is not an aliphatic-aromatic polyester. Preferably, the proportion of the aliphatic-aromatic polyester is 30% to 80% by weight, preferably 35% to 75% by weight, further preferably 40% to 70% by weight or likewise preferably 30% to 70% by weight and especially 45% to 70% by weight, based on the total weight of aliphatic-aromatic polyesters and the polymer that is not an aliphatic-aromatic polyester. Accordingly, the mass ratio of aliphatic-aromatic polyesters to the at least one further polymer that is different from aliphatic-aromatic polyesters is in the range from 30:70 to 99:1 or in the range from 30:70 to 80:20, in particular in the range from 35:65 to 75:25 and especially in the range from 40:60 to 70:30 or in the range from 30:70 to 70:30 and especially in the range from 45:55 to 70:30.

In particularly preferred embodiments, the wall material comprises or consists of a combination of
i) 30% to 80% by weight, preferably 35% to 75% by weight, further preferably 40% to 70% by weight and especially 45% to 70% by weight, based in each case on the total mass of the wall material, of at least one aliphatic-aromatic polyester selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
ii) 20% to 70% by weight, preferably 25% to 65% by weight, further preferably 30% to 60% by weight and especially 30% to 55% by weight, based in each case on the total mass of the wall material, of at least one aliphatic polyester selected from aliphatic polyesters, especially from polyhydroxy fatty acids including poly-C₆-C₁₂-lactones, polyhydroxyacetic acid, polylactic acid and aliphatic-aliphatic polyesters and mixtures thereof, and especially from polylactic acid, aliphatic-aliphatic polyesters and poly-C₆-C₁₂-lactones.

In specific embodiments, the wall material comprises or consists of a combination of
i) 30% to 80% by weight, preferably 35% to 75% by weight, further preferably 40% to 70% by weight and especially 45% to 70% by weight, based in each case on the total mass of the wall material, of at least one aliphatic-aromatic polyester selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
ii) 20% to 70% by weight, preferably 25% to 65% by weight, further preferably 30% to 60% by weight and especially 30% to 55% by weight, based in each case on the total mass of the wall material, of at least one aliphatic polyester selected from polycaprolactones, polylactic acid (PLA), polylactide glycolide, polybutylene succinate adipate, polybutylene succinate, polybutylene sebacate, polybutylene succinate sebacate.

In a specific embodiment, spherical microparticles are impregnated, wherein the spherical microparticles are selected from spherical microparticles consisting of a wall material composed of 30% to 80% by weight, preferably 35% to 75% by weight, further preferably 40% to 70% by weight and especially 45% to 70% by weight, based in each case on the total mass of the wall material, of PBSeT and 20% to 70% by weight, preferably 25% to 65% by weight, further preferably 30% to 60% by weight and especially 30% to 55% by weight, based in each case on the total mass of the wall material, of polycaprolactone, and spherical microparticles consisting of a wall material composed of 30% to 80% by weight, preferably 35% to 75% by weight, further preferably 40% to 70% by weight and especially 45% to 70% by weight, based in each case on the total mass of the wall material, of PBAT and 20% to 70% by weight, preferably 25% to 65% by weight, further preferably 30% to 60% by weight and especially 30% to 55% by weight, based in each case on the total mass of the wall material, of polycaprolactone. In a very specific embodiment, the spherical microparticles are selected from spherical microparticles consisting of a wall material composed of 55% by weight of PBAT and 45% by weight of polycaprolactone, based in each case on the total mass of the wall material, and spherical microparticles consisting of a polymer material composed of 55% by weight of PBSeT and 45% by weight of polycaprolactone, based in each case on the total mass of the wall material.

Preference is given to mixtures of an aliphatic-aromatic polyester with a further polymer which is not an aliphatic-aromatic polyester, especially the mixtures with aliphatic-aliphatic polyesters, in which the melting point of the aliphatic-aromatic polyester is at least 10° C., preferably at least 20° C., above the melting point of the further polymer, or the glass transition temperature of the aliphatic-aromatic polyester is at least 10° C., preferably at least 20° C., above the glass transition temperature of the further polymer. If the further polymer is an amorphous compound, the melting point of the aliphatic-aromatic polyester is at least 10° C., preferably at least 20° C., above the glass transition temperature of the further polymer.

Especially preferred microparticles for loading are the particles described in WO 2018/065481 and the prior European patent application 18166159.6, especially those described in the examples therein.

The person skilled in the art is able to produce the microparticles described in a manner known per se, for example by the procedure described in WO 2011/088229 or WO 2015/070172 and especially in WO 2018/065481 or in prior European patent application 18166159.6.

The microparticles intended for loading are typically produced by a process in which
a) a water-in-oil emulsion (w/o emulsion) is prepared from water or an aqueous solution of a pore former as discontinuous phase and a continuous phase comprising a solution of at least one polymer or polymer mixture suitable as a wall material, particularly comprising at least one polyester, especially at least one aliphatic-aromatic polyester, in a water-immiscible solvent,
b) the w/o emulsion obtained in a) is emulsified in water in the presence of a dispersant to give a w/o/w emulsion (water-in-oil-water emulsion) with droplets of average size 10-600 μm, and the water-immiscible solvent is removed at a temperature in the range from 20 to 80° C., preferably from 20 to 45° C.,
c) the microparticles formed in process step b) are separated off and optionally dried.

The microparticles are thus typically produced by removing the solvent in a w/o/w emulsion. In the first step, an emulsion of water droplets or droplets of the aqueous pore former solution is formed in the polyester solution. This w/o emulsion is in turn emulsified in water to give a w/o/w emulsion and the water-immiscible solvent is removed. Removal of the solvent makes the polymer or polymer mixture insoluble and it separates out at the surface of the water droplets or the aqueous pore former droplets. During this wall forming process, the pores are simultaneously formed, advantageously brought about by the pore former. Pore formers are, for example, compounds that release gas under the process conditions of step b).

Pore formers are typically agents that release a gas, e.g. CO₂, and are preferably selected from ammonium carbonate, sodium carbonate, ammonium hydrogen-carbonate, ammonium sulfate, ammonium oxalate, sodium hydrogencarbonate, ammonium carbamate and sodium carbamate.

Further suitable pore formers are water-soluble low molecular weight compounds that create an osmotic pressure. Removal of the water-insoluble solvent, on account of the concentration gradient that exists between the inner aqueous droplets with pore former and the outer aqueous disperse phase, builds up a concentration gradient which leads to migration of the water in the direction of the inner droplets and hence to the formation of pores. Such pore formers are preferably selected from sugars such as monosaccharides, disaccharides, oligosaccharides and polysaccharides, urea, inorganic alkali metal salts such as sodium chloride and inorganic alkaline earth metal salts such as magnesium sulfate and calcium chloride. Particular preference is given to glucose and sucrose and urea.

Further suitable pore formers are polymers that are soluble in both phases, for example polyethylene glycol (PEG) and polyvinylpyrrolidone (PVP). Since these polymers are soluble in both phases, they migrate from the aqueous phase into the oil phase owing to diffusion.

The processes for producing the spherical microparticles always lead to a population of microparticles, and therefore the term “composition of spherical microparticles” is also used.

In a preferred embodiment, the composition of the microparticles intended for loading is produced by the double emulsion method. This more preferably comprises the process steps specified herein, a), b) and c). In this way, spherical microparticles having the abovementioned particle sizes and pore contents are obtained.

### Process Step a)

For this purpose, the polymer or polymer mixture suitable as wall material is dissolved in a water-immiscible solvent.

In relation to process step a), “water-immiscible” means that the solvent has a solubility in water, at a temperature of 20° C. and a pressure of 1 bar, of ≤90 g/L. In addition, the water-immiscible solvent preferably has a boiling point of at least 30° C. According to the general knowledge of those skilled in the art, solvents are chemically inert to the substances to be dissolved therein; that is to say, they merely serve for dilution or dissolution. Free radically-polymerizable monomers are not solvents in the context of the invention.

Preference is given to aprotic non-polar and aprotic polar solvents or solvent mixtures, which have a water solubility of <90 g/L (at 20° C.). Preferred solvents are, for example, dichloromethane, chloroform, ethyl acetate, n-hexane, cyclohexane, methyl tert-butyl ether, pentane, diisopropyl ether and benzene, or mixtures of two or more of these solvents with one another. Dichloromethane is particularly preferred. Further suitable solvent mixtures are those that form an azeotrope having a boiling point within the range from 20 to 80° C. One example is the azeotrope of hexane and methyl ethyl ketone (MEK) in the weight ratio of 72:28.

In general, the polymer or polymer mixture suitable as wall material is used as a 1% to 50% by weight solution in the water-immiscible solvent. Preferably, the polymer solution thus prepared is a 2% to 30% by weight, especially 5% to 20% by weight, solution in the water-immiscible solvent.

According to the invention, an emulsion of a solution of at least one polymer suitable as wall material or of a polymer mixture is chosen. Preference is given to an emulsion of a solution of the polymer or polymer mixture suitable as wall material which is at least one polyester, particularly at least one aliphatic-aromatic polyester and especially a mixture of at least one aliphatic-aromatic polyester with a further polymer which is not an aliphatic-aromatic polyester and is especially an aliphatic-aliphatic polyester such as polylactic acid. If the wall material used is a mixture of polymers, the solution used to prepare the microparticles can be obtained by mixing the individual polymer solutions or be prepared by co-dissolving a mixture of polymers. The polymer or polymer mixture suitable as wall material is the wall material of the later microparticle. The wall material of the microparticles preferably has a solubility at 25° C. and 1 bar of at least 50 g/L in dichloromethane.

In a preferred embodiment, the continuous phase prepared in a) consists essentially of the solution of an aliphatic-aromatic polyester in a water-immiscible solvent. The continuous phase more preferably consists to an extent of at least 95% by weight, especially to an extent of at least 99% by weight, based on the continuous phase, of the solution of an aliphatic-aromatic polyester in a water-immiscible solvent. In other words, the continuous phase prepared in a) consists essentially, i.e. to an extent of at least 95% by weight, especially to an extent of at least 99% by weight, of the aliphatic-aromatic polyester and the water-immiscible solvent.

In a further particularly preferred embodiment of the process, the continuous phase prepared in a) comprises the aliphatic-aromatic polyester and at least one further dissolved polymer that is not an aliphatic-aromatic polyester and is especially selected from the aforementioned preferred or particularly preferred polymers and mixtures thereof. In this solution, the mass ratio of aliphatic-aromatic polyester to the at least one further polymer other than the aliphatic-aromatic polyesters is preferably in the range from 30:70 to 99:1 or in the range from 30:70 to 80:20, particularly in the range from 35:65 to 75:25 and especially in the range from 40:60 to 70:30 or in the range from 30:70 to 70:30 and especially in the range from 45:55 to 70:30.

With regard to the for preparation of the continuous polymers, the statements already made above in respect of the wall material are applicable in the same way.

In particularly preferred embodiments, the polymers used for preparation of the continuous phase comprise or consist of a combination of
i) at least one aliphatic-aromatic polyester selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
ii) at least one aliphatic polyester selected from aliphatic polyesters, especially from polyhydroxy fatty acids including poly-C₆-C₁₂-lactones, polyhydroxyacetic acid, polylactic acid and aliphatic-aliphatic polyesters and mixtures thereof, and specifically from polylactic acid, aliphatic-aliphatic polyesters and poly-C₆-C₁₂-lactones.

In very particularly preferred embodiments, the polymers used for preparation of the continuous phase comprise or consist of a combination of
i) at least one aliphatic-aromatic polyester selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
ii) at least one aliphatic polyester selected from polycaprolactones, polylactic acid (PLA), polylactide glycolide, polybutylene succinate adipate, polybutylene succinate, polybutylene sebacate, polybutylene succinate sebacate.

In particularly preferred embodiments, the polymers used for preparation of the continuous phase comprise or consist of a combination of
i) 30% to 80% by weight, preferably 35% to 75% by weight, further preferably 40% to 70% by weight and especially 45% to 70% by weight, based in each case on the total mass of the polymer combination used, of at least one aliphatic-aromatic polyester selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
ii) 20% to 70% by weight, preferably 25% to 65% by weight, further preferably 30% to 60% by weight and especially 30% to 55% by weight, based in each case on the total mass of the polymer combination used, of at least one aliphatic polyester selected from aliphatic polyesters, especially from polyhydroxy fatty acids including poly-C₆-C₁₂-lactones, polyhydroxyacetic acid, polylactic acid and aliphatic-aliphatic polyesters and mixtures thereof, and especially from polylactic acid, aliphatic-aliphatic polyesters and poly-C₆-C₁₂-lactones.

In specific embodiments, the polymers used for preparation of the continuous phase comprise or consist of a combination of
iii) 30% to 80% by weight, preferably 35% to 75% by weight, further preferably 40% to 70% by weight and especially 45% to 70% by weight, based in each case on the total mass of the polymer combination used, of at least one aliphatic-aromatic polyester selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
iv) 20% to 70% by weight, preferably 25% to 65% by weight, further preferably 30% to 60% by weight and especially 30% to 55% by weight, based in each case on the total mass of the polymer combination used, of at least one aliphatic polyester selected from polycaprolactones, polylactic acid (PLA), polylactide glycolide, polybutylene succinate adipate, polybutylene succinate, polybutylene sebacate, polybutylene succinate sebacate.

In a specific embodiment, the polymers used for preparation of the continuous phase consist of a combination of 30% to 80% by weight, preferably 35% to 75% by weight, further preferably 40% to 70% by weight and especially 45% to 70% by weight, based in each case on the total mass of the combination, of PBSeT and 20% to 70% by weight, preferably 25% to 65% by weight, further preferably 30% to 60% by weight and especially 30% to 55% by weight, based in each case on the total mass of the combination, of polycaprolactone, or of a combination of 30% to 80% by weight, preferably 35% to 75% by weight, further preferably 40% to 70% by weight and especially 45% to 70% by weight, based in each case on the total mass of the combination, of PBAT and 20% to 70% by weight, preferably 25% to 65% by weight, further preferably 30% to 60% by weight and especially 30% to 55% by weight, based in each case on the total mass of the combination, of polycaprolactone. In a very specific embodiment, the polymers used for preparation of the continuous phase consist of a combination of 55% by weight of PBAT and 45% by weight of polycaprolactone, based in each case on the total mass of the combination, or of a combination of 55% by weight PBSeT and 45% by weight of polycaprolactone, based in each case on the total mass of the combination.

Water or an aqueous solution of the pore former is emulsified in this polymer solution in process step a).

The aqueous solution of the pore former is preferably a 0.1% to 10% by weight aqueous solution of the pore former, especially of a pore former selected from ammonium hydrogencarbonate and ammonium carbonate. Particular preference is given to using ammonium carbonate, especially a 0.1% to 1% by weight solution of ammonium carbonate in water, as pore former solution.

0.1 to 10 parts by weight of the pore former, based on the 100 parts by weight of the sum total of the polymers that form the wall material, are used. The polymers that form the wall material consist of preferably at least one polyester or a mixture of two or more polyesters, or at least one polyester and at least one non-polyester polymer, especially of at least one aliphatic-aromatic polyester, at least one additional polymer other than an aliphatic-aromatic polyester, e.g. an aliphatic polyester, and optionally at least one further polymer. Preference is given to using 1 to 5 parts by weight, especially 1.3 to 3 parts by weight, of the pore former based on 100 parts by weight of the sum total of the polymers that form the wall material.

The emulsifying in process step a) is usually effected with the aid of a disperser, for example a rotor-stator or rotor-rotor disperser, or with the aid of a high-pressure disperser or high-pressure homogenizer or of an ultrasound homogenizer or of a toothed ring dispersing machine. More particularly, the aforementioned homogenizing or dispersing machines are suitable for production of the w/o emulsion, since these can introduce high shear energy into the system and hence small droplet sizes are obtained. The average droplet size, i.e. the D[4,3] value, of the emulsion droplets is generally 0.2 to 50 μm.

The w/o emulsion produced in process step a) can optionally be stabilized with one or more dispersants. Dispersants suitable for w/o emulsions are common knowledge and are mentioned for example in EP 2794085 and EP 3007815, the teaching of which is expressly incorporated by reference.

In place of or together with the aforementioned dispersants, for production of the w/o emulsion in step a) and for stabilization thereof, it is possible to use one or more emulsifiers preferably having an HLB value according to Griffin in the range from 2 to 10, especially in the range from 3 to 8. The HLB value (HLB=hydrophilic-lipophilic balance) according to Griffin (W. C. Griffin: Classification of surface active agents by HLB. In: J. Soc. Cosmet. Chem. 1, 1949, p. 311-326) is a dimensionless number between 0 and 20, that makes statements as to the water and oil solubility of a compound. Preference is given to nonionic emulsifiers having an HLB value according to Griffin in the range from 2 to 10, especially in the range from 3 to 8. Also suitable, however, are anionic and zwitterionic emulsifiers having an HLB value according to Griffin in the range from 2 to 10, especially in the range from 3 to 8.

In general, such emulsifiers will be used in an amount of 0.1% to 5% by weight, especially 0.5% to 4% by weight, based on the total weight of the emulsion produced in step a). In general, the emulsifier(s) will be added to the solution of the polymer or polymer mixture suitable as wall material in the water-immiscible solvent before water or an aqueous solution of the pore former is emulsified in this solution of the polymer or polymer mixture.

Examples of suitable emulsifiers having an HLB value according to Griffin in the range from 2 to 10 are:
sorbitan fatty acid esters, especially sorbitan mono-, di- and trifatty acid esters and mixtures thereof, such as sorbitan monostearate, sorbitan monooleate, sorbitan monolaurate, sorbitan tristearate, sorbitan sesquioleate, sorbitan dioleate, sorbitan trioleate;
fatty acid esters of glycerol or of polyglycerol, such as glycerol monostearate, glycerol distearate, glycerol monooleate, glycerol dioleate, glycerol monostearate monoacetate, glycerol monoacetate monooleate, polyglycerol polyricinoleate (E47S), for example the commercially available emulsifier PGPR 90,
lactyl esters of fatty acid monoesters of glycerol;
lecithins;
ethoxylated castor oils, ethoxylated hydrogenated castor oils having degrees of ethoxylation in the range from 2 to 20
ethoxylated and/or propoxylated C₁₂-C₂₂-alkanols having degrees of alkoxylation in the range from 2 to 10, e.g. stearyl alcohol ethoxylate having a degree of ethoxylation in the range from 2 to 5, stearyl alcohol ethoxylate-co-propoxylate having degrees of alkoxylation in the range from 2 to 8, isotridecyl ethoxylates having degrees of ethoxylation in the range from 2 to 3 and isotridecyl ethoxylate-co-propoxylate having degrees of alkoxylation in the range from 2 to 5,
ethoxylated and/or propoxylated C₄-C₁₆-alkylphenols having degrees of alkoxylation in the range from 2 to 10, e.g. nonylphenol ethoxylate having degrees of ethoxylation in the range from 2 to 5 and octylphenol ethoxylate having degrees of ethoxylation in the range from 2 to 5.

### Process Step b)

The emulsifying of the w/o emulsion in water to give the w/o/w emulsion in process step b) is effected by stirring or shearing in the presence of a dispersant. It is possible here to meter an aqueous solution of the dispersant into the w/o emulsion. The dispersant is preferably initially charged in the form of an aqueous solution and the w/o emulsion is metered in. Depending on the energy input, it is possible to control the droplet size. Furthermore, the dispersant described below influences the size of the emulsion droplets in equilibrium.

The concentration of the dispersant in the aqueous dispersant solution is typically in the range from 0.1% to 8.0% by weight, particularly in the range from 0.3% to 5.0% by weight and especially in the range from 0.5% to 4.0% by weight, based on the total weight of the aqueous solution.

The weight ratio of w/o emulsion provided in step a) to water, preferably in the form of the aqueous dispersant solution, is typically in the range from 15:85 to 55:45, particularly in the range from 25:75 to 50:50, and especially in the range from 30:70 to 45:55.

In step b), the amount of dispersant used is typically at least 0.1% by weight, especially at least 0.2% by weight, based on the total weight of the w/o/w emulsion, and is particularly in the range from 0.1% to 2% by weight and especially in the range from 0.2% to 1% by weight, based on the total weight of the w/o/w emulsion.

Larger droplets having an average droplet size D[4,3] in the range from 100 to 600 μm are obtained by means of customary stirrers, whereas average droplet sizes D[4,3] of below 100 μm are achieved by means of apparatuses for generating a high shear field. It is also possible to introduce sufficient shear energy by vigorous stirring that average droplet sizes with D[4,3] values in the range from 1 to <100 μm, preferably of 5 to 50 μm, are achieved. Should even higher shear energy input be intended, it may be advantageous to use apparatuses for generating a high shear field.

Suitable stirrer types include propeller stirrers, impeller stirrers, disk stirrers, paddle stirrers, anchor stirrers, pitched-blade stirrers, cross-beam stirrers, helical stirrers, screw stirrers and others.

Suitable apparatuses for generating a high shear field are comminutors operating by the rotor-stator principle, such as toothed ring dispersing machines, and also colloid and corundum disk mills and high-pressure and ultrasound homogenizers. Preference is given to the use of the toothed ring dispersing machines operating by the rotor-stator principle for generating the shear field. The diameter of the rotors and stators is typically in the range between 2 and 40 cm, depending on machine size and dispersing performance. The speed of rotation of such dispersing machines is generally in the range from 500 to 20 000 rpm (revolutions per minute), depending on the construction type. Of course, machines with large rotor diameters rotate at the lower end of the rotation speed range, while machines with small rotor diameters are usually operated at the upper end of the rotation speed range. The distance of the rotating parts from the stationary parts of the dispersing tool is generally 0.1 to 3 mm.

In a preferred embodiment, the final size of the emulsion droplets of the w/o/w emulsion should be an average diameter D[4,3] (determined by means of light scattering) of 100 to 600 μm. This final size is generally achieved just by stirring.

In a likewise preferred embodiment, the final size of the emulsion droplets of the w/o/w emulsion should have an average diameter of 10 to 100 μm, preferably 10 to 30 μm. This final size is typically achieved by means of shearing.

The shear energy introduced can be directly derived from the power consumption of the apparatus for generating a shear field, taking account of the heat loss. Thus, the shear energy input into the w/o/w emulsion is preferably 250 to 25 000 watts h/m³ batch size. Particular preference is given to an energy input of 500 to 15 000, especially 800 to 10 000, watts h/m³ batch size, calculated based on the motor current. The w/o/w emulsion is produced in the presence of at least one dispersant. In one embodiment, the w/o/w emulsion can be produced in the presence of a mixture of different dispersants. Likewise, it is also possible to employ just one dispersant. Suitable dispersants are, for example, cellulose derivatives such as hydroxyethyl cellulose, methyl hydroxyethyl cellulose, methyl cellulose and carboxymethyl cellulose, polyvinylpyrrolidone, copolymers of vinylpyrrolidone, gelatin, gum arabic, xanthan, casein, polyethylene glycols, and partly or fully hydrolyzed polyvinyl acetates (polyvinyl alcohols), and also methyl hydroxypropyl cellulose, and mixtures of the above. Preferred dispersants are partly or fully hydrolyzed polyvinyl acetates (polyvinyl alcohols) and also methyl hydroxy(C₁-C₄)alkyl celluloses and mixtures thereof. Particular preference is given to partly hydrolyzed polyvinyl acetates, which are also termed partly hydrolyzed polyvinyl alcohols (PVAs), preferably those with a degree of hydrolysis of 79% to 99.9%. In addition, PVA copolymers, as described in WO 2015/165836, are also suitable.

Methyl hydroxy(C₁-C₄)alkyl celluloses are understood to mean methyl hydroxy(C₁-C₄)alkyl celluloses of a wide variety of degrees of methylation and also degrees of alkoxylation.

The preferred methyl hydroxy(C₁-C₄)alkyl celluloses have an average degree of substitution DS of 1.1 to 2.5 and a molar degree of substitution MS of 0.03 to 0.9.

Suitable methyl hydroxy(C₁-C₄)alkyl celluloses are for example methyl hydroxyethyl cellulose or methyl hydroxypropyl cellulose.

Methyl hydroxypropyl cellulose is a particularly preferred dispersant.

Very particularly preferred dispersants are polyvinyl alcohols, especially polyvinyl alcohols having a degree of hydrolysis of 79% to 99.9%. A specific dispersant for step b) is a carboxy-modified anionic PVA having a carboxyl group content of 1 to 6 mol % and a degree of hydrolysis of 85 to 90 mol %, and very particularly a carboxy-modified anionic PVA of which the 4% by weight aqueous solution at 20° C. has a viscosity of 20.0 to 30.0 mPa·s.

In order to stabilize the w/o/w emulsion, the dispersant is especially added to the aqueous phase. The concentration of the dispersant in the aqueous phase is typically in the range from 0.1% to 8.0% by weight, particularly in the range from 0.3% to 5.0% by weight and especially in the range from 0.5% to 4.0% by weight, based on the total weight of the aqueous phase. The weight ratio of w/o emulsion provided in step a) to the aqueous phase comprising the dispersant is typically in the range from 15:85 to 55:45, particularly in the range from 25:75 to 50:50, and especially in the range from 30:70 to 45:55.

In a preferred embodiment, carboxy-modified anionic PVA (having a degree of hydrolysis of 85 to 90 mol % and a viscosity of 20.0 to 30.0 mPa-s (4% strength by weight aqueous solution at 20° C.) and a proportion of carboxyl groups of 1 to 6 mol %) is used as 0.1% to 8.0% strength by weight aqueous solution, particularly as 0.1% to 5.0% strength by weight aqueous solution and especially as 0.3% to 4.0% strength by weight aqueous solution. Aqueous solutions having a PVA content of 0.3% to 2.5% by weight, especially having a PVA content of 0.5% to 1.5% by weight can likewise be used.

In a preferred process variant, in process step b), the emulsification to give the w/o/w emulsion is effected with a stirrer at a stirring speed of 5000 to 15 000 rpm over a period of 1 to 30 minutes. The droplets produced thereby have a mean diameter of 0.2 to 30 μm.

In a further preferred process variant, the emulsion is prepared at a stirring speed of to 1000 rpm over a period of 1 to 30 minutes. The average diameter of the droplets produced thereby is preferably 100 to 600 μm.

During the emulsification, and optionally thereafter, the mixture is kept at a temperature in the range from 10 to 80° C. The temperature of the mixture is preferably selected such that it is below the glass transition temperature of the lowest softening amorphous polymer or below the melting point of the lowest melting crystalline polymer of the composition that forms the wall material. Higher temperatures are possible, but they may lead to partial closure of the pores over too long a period. The mixture is preferably kept at a temperature in the range from 20 to 45° C., especially from 20 to <40° C. Optionally, a vacuum may additionally be applied. For example, it is possible to work in the range from 600 to 800 mbar or below 200 mbar. These measures, both the stirring/shearing and the temperature and any vacuum applied, have the effect that the water-immiscible solvent evaporates and the microparticles remain in the form of an aqueous suspension.

Both measures, the stirring/shearing and also the temperature, lead to the water-immiscible solvent of the at least one aliphatic-aromatic polyester evaporating and the microparticles being left behind.

Provided that the solvent is one having a vapor pressure ≥450 hPa at 20° C., it is sufficient to stir the w/o/w emulsion obtained in b) at room temperature, 20° C. Depending on the amount of the solvent and the ambient temperature, such an operation lasts for a few hours. Depending on the solvent, it is possible to facilitate the removal of the solvent by raising the temperature to a temperature of up to 80° C. and/or by applying a vacuum.

In the course of the removal of the water-immiscible solvent, pore formation is observed in the walls of the microparticles.

The microparticles formed by removal of the water-immiscible solvent are removed in process step c) and preferably dried. “Dried” is understood to mean that the microparticles comprise a residual amount of water of ≤5% by weight, preferably ≤1% by weight, based on the microparticles. The drying may for example be carried out in a stream of air and/or by applying a vacuum, optionally in each case with heating. This is accomplished, depending on the size of the microparticles, by means of convective driers such as spray driers, fluidized bed and cyclone driers, contact driers such as pan driers, paddle driers, contact belt driers, vacuum drying cabinet or radiative driers such as infrared rotary tube driers and microwave mixing driers.

One feature of the spherical microparticles thus obtained is that they are easy to fill, for example by suspending them in a solution.

A specifically preferred process for producing the microparticles is described in WO 2018/065481.

In the process of the invention, the microparticles are laden with at least one organic active.

Preferably, the organic active of low molecular weight is liquid at 22° C. and 1013 mbar or has a melting point below 100° C. Preference is given particularly to actives that are liquid at 22° C. and 1013 mbar.

In the process of the invention, it is possible to use either one active or a mixture of actives. This may be a mixture of actives from one class or a mixture of actives from different classes.

In the process of the invention, the microparticles may also be laden with a mixture of different actives.

In a preferred group of embodiments, the organic active of low molecular weight is an aroma chemical, especially an aroma chemical which is liquid at 22° C. and 1013 mbar or a mixture of two or more aroma chemicals which is liquid at 22° C. and 1013 mbar.

Preferred aroma chemicals are hydrophobic and, especially at 25° C., have a water solubility in deionized water of not more than 100 mg/L.

The term “aroma chemical” is understood by the person skilled in the art to mean organic compounds usable as “odorant” and/or as “flavoring”. In the context of the present invention, “odorant” is understood to mean natural or synthetic substances having intrinsic odor. In the context of the present invention, “flavoring” is understood to mean natural or synthetic substances having intrinsic flavor. In the context of the present invention, “odor” or “olfactory perception” is the interpretation of the sensory stimuli which are sent from the chemoreceptors in the nose or other olfactory organs to the brain of a living being. The odor can be a result of sensory perception of the nose of odorant, which occurs during inhalation. In this case, the air serves as odor carrier.

Preferred aroma chemicals for loading of the microparticles are selected, for example, from the following compounds:

alpha-hexylcinnamaldehyde, 2-phenoxyethyl isobutyrate (Phenirat¹), dihydromyrcenol (2,6-dimethyl-7-octen-2-ol), methyl dihydrojasmonate (preferably having a cis isomer content of more than 60% by weight) (Hedione⁹, Hedione HC⁹), 4,6,6,7,8,8-hexamethyl-1,3,4,6,7,8-hexahydrocyclopenta[g]benzopyran (Galaxolide³), tetrahydrolinalool (3,7-dimethyloctan-3-ol), ethyl linalool, benzyl salicylate, 2-methyl-3-(4-tert-butylphenyl)propanal (Lilial²), cinnamyl alcohol, 4,7-methano-3a,4,5,6,7,7a-hexahydro-5-indenyl acetate and/or 4,7-methano-3a,4,5,6,7,7a-hexahydro-6-indenyl acetate (Herbaflorat¹), citronellol, citronellyl acetate, tetrahydrogeraniol, vanillin, linalyl acetate, styrenyl acetate (1-phenylethyl acetate), octahydro-2,3,8,8-tetramethyl-2-acetonaphthone and/or 2-acetyl-1,2,3,4,6,7,8-octahydro-2,3,8,8-tetramethylnaphthalene (Iso E Super³), hexyl salicylate, 4-tert-butylcyclohexyl acetate (Oryclone¹), 2-tert-butylcyclohexyl acetate (Agrumex HC¹), alpha-ionone (4-(2,2,6-trimethyl-2-cyclohexen-1-yl)-3-buten-2-one), n-alpha-methylionone, alpha-isomethylionone, coumarin, terpinyl acetate, 2-phenylethyl alcohol, 4-(4-hydroxy-4-methylpentyl)-3-cyclohexenecarboxaldehyde (Lyral³), alpha-amylcinnamaldehyde, ethylene brassylate, (E)- and/or (Z)-3-methylcyclopentadec-5-enone (Muscenone⁹), 15-pentadec-11-enolide and/or 15-pentadec-12-enolide (Globalide¹), 15-cyclopentadecanolide (Macrolide¹), 1-(5,6,7,8-tetrahydro-3,5,5,6,8,8-hexamethyl-2-naphthalenyl)ethanone (Tonalide¹⁰), 2-isobutyl-4-methyltetrahydro-2H-pyran-4-ol (Florol⁹), 2-ethyl-4-(2,2,3-trimethyl-3-cyclopenten-1-yl)-2-buten-1-ol (Sandolene¹), cis-3-hexenyl acetate, trans-3-hexenyl acetate, trans-2-cis-6-nonadienol, 2,4-dimethyl-3-cyclohexenecarboxaldehyde (Vertocitral¹), 2,4,4,7-tetramethyloct-6-en-3-one (Claritone¹), 2,6-dimethyl-5-hepten-1-al (Melonal²), borneol, 3-(3-isopropylphenyl)butanal (Florhydral²), 2-methyl-3-(3,4-methylenedioxyphenyl)propanal (Helional³), 3-(4-ethylphenyl)-2,2-dimethylpropanal (Florazon¹), tetrahydro-2-isobutyl-4-methyl-2H-pyran (Dihydrorosenon⁴), 1,4-bis(ethoxymethyl)cyclohexane (Vertofruct⁴), L-isopulegol (1R,2S,5R)-2-isopropenyl-5-methylcyclohexanol, pyranyl acetate (2-isobutyl-4-methyltetrahydropyran-4-yl acetate), nerol ((Z)-2,6-dimethyl-2,6-octadien-8-ol), neryl acetate, 7-methyl-2H-1,5-benzodioxepin-3(4H)-one (Calone¹⁹⁵¹⁵), 3,3,5-trimethylcyclohexyl acetate (preferably with a content of cis isomers of 70% by weight) or more and 2,5,5-trimethyl-1,2,3,4,4a,5,6,7-octahydronaphthalen-2-ol (Ambrinol S¹), tetrahydro-4-methyl-2-(2-methylpropenyl)-2H-pyran (rose oxide), 4-methyl-2-(2-methylpropyl)oxane or 4-methyl-2-(2-methylpropyl)-2H-pyran (Dihydrorosan⁴), prenyl acetate (=3-methylbut-2-enyl acetate), isoamyl acetate, dihydromyrcenol (2,6-dimethyloct-7-en-2-ol) and methylheptenone (6-methylhept-5-en-2-one) and mixtures thereof, and also mixtures thereof with one or more other aromas.

In the context of the present invention, the aforementioned aromas or odorants are accordingly preferably combined with mixtures of the invention.

If trade names are specified above, these refer to the following sources:

¹ trade name of Symrise GmbH, Germany;
² trade name of Givaudan AG, Switzerland;
³ trade name of International Flavors & Fragrances Inc., USA;
⁴ trade name of BASF SE;
⁵ trade name of Danisco Seillans S.A., France;
⁹ trade name of Firmenich S.A., Switzerland;
¹⁰ trade name of PFW Aroma Chemicals B.V., the Netherlands.

More particularly, the advantages of the invention are manifested in the case of aroma chemicals that are selected from volatile fragrances and aroma mixtures comprising at least one volatile fragrance. Volatile fragrances are understood to mean fragrances having a high vapor pressure at room temperature. A fragrance is considered to be a volatile fragrance especially when it has the following property: If a droplet of the volatile fragrance is applied to a strip of paper and left to evaporate off under ambient conditions at room temperature (22° C.), its odor is no longer perceptible to an experienced perfumer no longer than 2 hours after application. The volatile fragrances especially include the following compounds: rose oxide (tetrahydro-4-methyl-2-(2-methylpropenyl)-2H-pyran), 4-methyl-2-(2-methylpropyl)oxane or 4-methyl-2-(2-methylpropyl)-2H-pyran (Dihydrorosan®), prenyl acetate (=3-methylbut-2-enyl acetate), isoamyl acetate, dihydromyrcenol (2,6-dimethyloct-7-en-2-ol) and methylheptenone (6-methylhept-5-en-2-one). If an aroma mixture comprising at least one volatile fragrance is used for loading, the proportion of the volatile fragrance is generally at least 1% by weight, especially at least 5% by weight, for example 1% to 99% by weight, especially 5% to 95% by weight, based on the total weight of the aroma chemical mixture used for loading.

Further odorants or aroma chemicals with which the odorants mentioned can be combined to give an odorant composition can be found, for example, in S. Arctander, Perfume and Flavor Chemicals, Vol. I and II, Montclair, N. J., 1969, Author's edition or K. Bauer, D. Garbe and H. Surburg, Common Fragrance and Flavor Materials, 4th. Ed., Wiley-VCH, Weinheim 2001. Specifically, the following may be mentioned:

extracts from natural raw materials such as essential oils, concretes, absolutes, resins, resinoids, balsams, tinctures, for example
ambra tincture; amyris oil; angelica seed oil; angelica root oil; anise oil; valerian oil; basil oil; tree moss absolute; bay oil; mugwort oil; benzoin resin; bergamot oil; beeswax absolute; birch tar oil; bitter almond oil; savory oil; bucco leaf oil; cabreuva oil; cade oil; calamus oil; camphor oil; cananga oil; cardamom oil; cascarilla oil; cassia oil; cassie absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill oil; dill seed oil; eau de brouts absolute; oakmoss absolute; elemi oil; estragon oil; eucalyptus citriodora oil; eucalyptus oil; fennel oil; spruce needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; camellia oil blue; camellia oil roman; carrot seed oil; cascarilla oil; pine needle oil; spearmint oil; cumin oil; labdanum oil; labdanum absolute; labdanum resin; lavandin absolute; lavandin oil; lavender absolute; lavender oil; lemon grass oil; lovage oil; lime oil distilled; lime oil pressed; linalool oil; litsea cubeba oil; laurel leaf oil; macis oil; marjoram oil; mandarin oil; massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanaxoil; orange blossom absolute; orange oil; oregano oil; palmarosa oil; patchouli oil; perilla oil; Peruvian balsam oil; parsley leaf oil; parsley seed oil; petitgrain oil; peppermint oil; pepper oil; allspice oil; pine oil; poley oil; rose absolute; rosewood oil; rose oil; rosemary oil; sage oil dalmatian; sage oil Spanish; sandalwood oil; celery seed oil; spike lavender oil; star anise oil; styrax oil; tagetes oil; fir needle oil; tea tree oil; turpentine oil; thyme oil; tolu balsam; tonka absolute; tuberose absolute; vanilla extract; violet leaf absolute; verbena oil; vetiveroil; juniper berry oil; wine yeast oil; vermouth oil; wintergreen oil; ylang oil; hyssop oil; civet absolute; cinnamon leaf oil; cinnamon bark oil; and fractions thereof or ingredients isolated therefrom;

Individual odorants are, for example, those from the group of
the hydrocarbons, for example 3-carene; alpha-pinene; beta-pinene; alpha-terpinene; gamma-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;
the aliphatic alcohols, for example hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
the aliphatic aldehydes and acetals thereof, for example hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethylacetal; 1,1-dimethoxy-2,2,5-trimethyl-4-hexene; citronellyloxyacetaldehyde; (E/Z)-1-(1-methoxypropoxy)-3-hexene; the aliphatic ketones and oximes thereof, for example 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
the aliphatic sulfur-containing compounds, for example 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
the aliphatic nitriles, for example 2-nonenenitrile; 2-undecenenitrile; 2-tridecenenitrile; 3,12-tridecadienenitrile; 3,7-dimethyl-2,6-octadienenitrile; 3,7-dimethyl-6-octenenitrile;
the esters of aliphatic carboxylic acids, for example (E)- and (Z)-3-hexenyl formate; ethyl acetoacetate; isoamyl acetate; hexyl acetate; 3,5,5-tri methyl hexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; (E/Z)-ethyl 2,4-decadienoate;
methyl 2-octynoate; methyl 2-nonynoate; allyl 2-isoamyloxyacetate; methyl 3,7-dimethyl-2,6-octadienoate; 4-methyl-2-pentyl crotonate;
the acyclic terpene alcohols, for example geraniol; nerol; linalool; lavandulol; nerolidol; farnesol; tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2-butenoates thereof;
the acyclic terpene aldehydes and ketones, for example geranial; neral; citronellal; 7-hydroxy-3,7-dimethyloctanal; 7-methoxy-3,7-dimethyloctanal; 2,6,10-trimethyl-9-undecenal; geranyl acetone; and also the dimethyl and diethyl acetals of geranial, neral, 7-hydroxy-3,7-dimethyloctanal; the cyclic terpene alcohols, for example menthol; isopulegol; alpha-terpineol; terpineol-4; menthan-8-ol; menthan-1-ol; menthan-7-ol; borneol; isoborneol; linalool oxide; nopol; cedrol; ambrinol; vetiverol; guajol; and the formates, acetates, propionates, isobutyrates, butyrates, isovalerates, pentanoates, hexanoates, crotonates, tiglinates and 3-methyl-2-butenoates thereof;
the cyclic terpene aldehydes and ketones, for example menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalene-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedar wood oil (methyl cedryl ketone); the cyclic alcohols, for example 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
the cycloaliphatic alcohols, for example alpha-3,3-trimethylcyclohexylmethanol; 1-(4-isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
the cyclic and cycloaliphatic ethers, for example cineol; cedryl methyl ether; cyclododecyl methyl ether; 1,1-dimethoxycyclododecane; 1,4-bis(ethoxymethyl)cyclohexane; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
the cyclic and macrocyclic ketones, for example 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; cis-3-methylpent-2-en-1-ylcyclopent-2-en-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; cyclohexadec-5-en-1-one; 6,7-dihydro-1,1,2,3,3-pentamethyl-4(5H)-indanone; 8-cyclohexadecen-1-one; 7-cyclohexadecen-1-one; (7/8)-cyclohexadecen-1-one; 9-cycloheptadecen-1-one; cyclopentadecanone; cyclohexadecanone;
the cycloaliphatic aldehydes, for example 2,4-dimethyl-3-cyclohexene-carbaldehyde; 2-methyl-4-(2,2,6-trimethylcyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;
the cycloaliphatic ketones, for example 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl (2,4-dimethyl-3-cyclohexen-1-yl) ketone;
the esters of cyclic alcohols, for example 2-tert-butylcyclohexyl acetate; A-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or -6-indenyl acetate; 4,7-methano-3a,4,5,6,7,7a-hexahydro-5- or -6-indenyl propionate; 4,7-methano-3a,4,5,6,7,7a-hexahydro-5- or -6-indenyl isobutyrate; 4,7-methanooctahydro-5- or -6-indenyl acetate;
the esters of cycloaliphatic alcohols, for example 1-cyclohexylethyl crotonate;
the esters of cycloaliphatic carboxylic acids, for example allyl 3-cyclohexyl-propionate; allyl cyclohexyloxyacetate; cis- and trans-methyl dihydrojasmonate; cis- and trans-methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolane-2-acetate;
the araliphatic alcohols, for example benzyl alcohol; 1-phenylethyl alcohol, 2-phenylethyl alcohol, 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenyl-pentanol; 3-phenyl-2-propen-1-ol; 4-methoxybenzyl alcohol; 1-(4-isopropyl-phenyl)ethanol;
the esters of araliphatic alcohols and aliphatic carboxylic acids, for example benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenyl-ethyl butyrate; cinnamyl acetate; 2-phenoxyethyl isobutyrate; 4-methoxybenzyl acetate;
the araliphatic ethers, for example 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;
the aromatic and araliphatic aldehydes, for example benzaldehyde; phenylacetaldehyde; 3-phenylpropanal; hydratropaaldehyde; 4-methylbenz-aldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butyl phenyl) propanal; 2-methyl-3-(4-isobutylphenyl)propanal; 3-(4-tert-butylphenyl) propanal; cinnamaldehyde; alpha-butylcinnamaldehyde; alpha-amylcinnamaldehyde; alpha-hexylcinnamaldehyde; 3-methyl-5-phenylpentanal; 4-methoxy-benzaldehyde; 4-hydroxy-3-methoxy-benzaldehyde; 4-hydroxy-3-ethoxybenz-aldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl) propanal;
the aromatic and araliphatic ketones, for example acetophenone; 4-methyl-acetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl) ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;
the aromatic and araliphatic carboxylic acids and esters thereof, for example benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxy-acetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
the nitrogen-containing aromatic compounds, for example 2,4,6-trinitro-1,3-dimethyl-5-tert-butylbenzene; 3,5-dinitro-2,6-dimethyl-4-tert-butylacetophenone; cinnamonitrile; 3-methyl-5-phenyl-2-pentenonitrile; 3-methyl-5-phenylpentano-nitrile; methyl anthranilate; methyl N-methylanthranilate; Schiff's bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropyl-quinoline; 6-isobutylquinoline; 6-sec-butylquinoline; 2-(3-phenylpropyl)pyridine; indole; skatole; 2-methoxy-3-isopropylpyrazine; 2-isobutyl-3-methoxypyrazine;
the phenols, phenyl ethers and phenyl esters, for example estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenyl-acetate;
the heterocyclic compounds, for example 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
the lactones, for example 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

In addition, suitable aroma chemicals are macrocyclic carbaldehyde compounds as described in WO 2016/050836.

Particular preference is given to mixtures of L-menthol and/or DL-menthol, L-menthone, L-menthyl acetate, or L-isopulegol, which are highly sought-after as analogs or substitutes for what are referred to as synthetic dementholized oils (DMOs). The mixtures of these minty compositions are preferably used in the ratio of L-menthol or DL-menthol 20-40% by weight, L-menthone 20-40% and L-menthyl acetate 0-20%, or in the ratio of 20-40% by weight, L-menthone 20-40% and L-isopulegol 0-20%.

The aforementioned aromas and aroma mixtures can be used as such or in a solvent which in itself is not an aroma. Typical solvents for aromas are especially those having a boiling point at standard pressure above 150° C. and which do not dissolve the wall material, e.g. diols such as propanediol and dipropylene glycol, C₈-C₂₂ fatty acid C₁-C₁₀-alkyl esters such as isopropyl myristate, di-C₆-C₁₀-alkyl ethers, e.g. dicapryl ether (Cetiol® OE from BASF SE), di-C₁-C₁₀-alkyl esters of aliphatic, aromatic or cycloaliphatic di- or tricarboxylic acids, for example dialkyl phthalates such as dimethyl and diethyl phthalate and mixtures thereof, dialkyl hexahydrophthalates, e.g. dimethyl cyclohexane-1,2-dicarboxylate, diethyl cyclohexane-1,2-dicarboxylate and diisononyl 1,2-cyclohexanedicarboxylate, and dialkyl adipates, such as dibutyl adipate (e.g. Cetiol® B from BASF SE), C₈-C₂₂ fatty acid triglycerides, e.g. vegetable oils or cosmetic oils such as octanoyl/decanoyltriglyceride (e.g. the commercial product Myritol® 318 from BASF SE), dimethyl sulfoxide and white oils.

In a further group of embodiments, the organic active of low molecular weight is an active pharmaceutical ingredient, API for short. Active pharmaceutical ingredients are typically active therapeutic ingredients, active diagnostic ingredients and active prophylactic ingredients, and corresponding combinations of active ingredients. The active pharmaceutical ingredient(s) may be in an amorphous state, a crystalline state or a mixture thereof. The active pharmaceutical ingredient(s) may be labelled with a detectable label such as a fluorescent label, a radioactive label or an enzymatic or chromatographically detectable species, and be used as a mixture with this label for loading of the microparticles.

The API may have a high water solubility, for example a water solubility in deionized water of more than 10 mg/mL at 25° C. It is also possible to use active pharmaceutical ingredients having low water solubility as actives, for example those having a water solubility in deionized water of less than 10 mg/mL at 25° C.

Preferred active therapeutic, diagnostic and prophylactic ingredients are those APIs that are suitable for parenteral administration. Representative examples of suitable APIs are the following categories and examples of APIs and alternative forms of these APIs, such as alternative salt forms, free acid forms, free base forms and hydrates:

analgesics/antipyretics; antiasthmatic drugs; antibiotics; antidepressants; antidiabetic drugs; antiphlogistics/inflammation inhibitors; antihypertensives; inflammation inhibitors; antineoplastics; antianxiety drugs; immunosuppressants; antimigraine drugs; tranquilizers/hypnotics; antitanginal drugs; antipsychotic drugs; antimanic drugs; antiarrhythmics; antiarthritic drugs; antigout drugs; anticoagulants; thrombolytic drugs; antifibrinolytic drugs; hemorheological drugs; antiplatelet drugs/thrombocyte aggregation inhibitors; anticonvulsives; anti-Parkinson's drugs; antihistamines/anti-pruritics; drugs for calcium regulation; antibacterial drugs; antiviral drugs; antimicrobial drugs; antiinfectives; bronchodilators; corticosteroids; steroidal compounds and hormones; hypoglycemic drugs; hypolipedemic drugs; proteins; nucleic acids; drugs useful for the stimulation of erythropoiesis; antiulcer drugs/antireflux drugs; antinausea drugs/antiemetics; oil-soluble vitamins and other medicaments.

Suitable active pharmaceutical ingredients are mentioned, for example, in WO 2007/070852, especially on pages 15 to 19. In addition, suitable active ingredients and drugs are listed in Martindale: The Extra Pharmacopoeia, 30th edition, The Pharmaceutical Press, London 1993.

In a further group of embodiments, the organic active of low molecular weight is an organic crop protecting agent. Organic crop protecting agents for loading of the microparticles are, for example, pesticides, especially selected from the group consisting of fungicides, insecticides, nematicides and herbicides, but also safeners, and growth regulators which can be used for loading of the microparticles also as mixtures, for example as mixtures of two or more herbicides, mixtures of two or more fungicides, mixtures of two or more insecticides, mixtures of insecticides and fungicides, mixtures of one or more herbicides with a safener, and mixtures of one or more fungicides with a safener.

Typically, the pesticides are liquid or solid at 20° C. and 1013 mbar and are normally nonvolatile. The vapor pressure is typically below 0.1 mbar at 20° C., especially below 0.01 mbar. Crop protecting agents particularly suitable for loading are hydrophobic and, especially at 25° C., have a water solubility in deionized water of not more than 10 g/L and especially not more than 1 g/L.

Crop protecting agents are known to those skilled in the art, for example from The Pesticide Manual, 17th edition, The British Crop Protection Council, London, 2015. Suitable crop protecting agents are listed, especially, in WO 2018/019629 on pages 10 to 15.

Examples of suitable insecticides are compounds from the classes of the carbamates, organophosphates, organochlorine insecticides, phenylpyrazoles, pyrethroids, neonicotinoids, spinosins, avermectins, milbemycins, juvenile hormone analogs, alkyl halides, organotin compounds, nereistoxin analogs, benzoylureas, diacylhydrazines, METI acaricides, and unclassified insecticides such as chloropicrin, pymetrozine, flonicamid, clofentezin, hexythiazox, etoxazole, diafenthiuron, propargite, tetradifon, chlorofenapyr, DNOC, buprofezine, cyromazine, amitraz, hydramethylnon, acequinocyl, fluacrypyrim, rotenone, or the agriculturally acceptable salts and derivatives thereof.

Examples of suitable fungicides are compounds from the classes of the dinitroanilines, allylamines, anilinopyrimidines, antibiotic fungicides, aromatic hydrocarbons, benzenesulfonamides, benzimidazoles, benzisothiazoles, benzophenones, benzothiadiazoles, benzotriazines, benzyl carbamates, carbamates, carboxamides, carboxylic acid diamides, chloronitriles, cyanoacetamide oximes, cyanoimidazoles, cyclopropanecarboxamides, dicarboximides, dihydrodioxazines, dinitrophenyl crotonates, dithiocarbamates, dithiolanes, ethylphosphonates, ethylaminothiazolecarboxamides, guanidines, hydroxy(2-amino)pyrimidines, hydroxyanilides, imidazoles, imidazolinones, isobenzofuranones, methoxyacrylates, methoxycarbamates, morpholines, N-phenylcarbamates, oxazolidinediones, oximinoacetates, oximinoacetamides, peptidylpyrimidine nucleosides, phenylacetamides, phenylamides, phenylpyrroles, phenylureas, phosphonates, phosphorothioates, phthalamic acids, phthalimides, piperazines, piperidines, propionamides, pyridazinones, pyridines, pyridinylmethylbenzamides, pyrimidineamines, pyrimidines, pyrimidinone hydrazones, pyrroloquinolinones, quinazolinones, quinolines, quinones, sulfamides, sulfamoyltriazoles, thiazolecarboxamides, thiocarbamates, thiophanates, thiophenecarboxamides, toluamides, triphenyltin compounds, triazines, triazoles and the agriculturally acceptable salts and derivatives thereof.

Examples of suitable fungicides are compounds from the classes of the acetamides, amides, aryloxyphenoxypropionates, benzamides, benzofurans, benzoic acids, benzothiadiazinones, bipyridylium salts, carbamates, chloroacetamides, chlorocarboxylic acids, cyclohexanediones, dinitroanilines, dinitrophenols, diphenyl ethers, glycines, imidazolinones, isoxazoles, isoxazolidinones, nitriles, N-phenylphthalimides, oxadiazoles, oxazolidinediones, oxyacetamides, phenoxycarboxylic acids, phenyl carbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl (thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolocarboxamides, triazolopyrimidines, triketones, uracils, ureas and the agriculturally acceptable salts and derivatives thereof.

In a specific subgroup of this embodiment, the crop protecting agent is a crop protecting agent which is liquid at 22° C. and 1013 mbar or a mixture of two or more crop protecting agents which is liquid at 22° C. and 1013 mbar. Examples of room temperature liquid active ingredients are dimethenamid, especially the enantiomer thereof dimethenamid-P, clomazone, metolachlor, especially the enantiomer thereof S-metolachlor. In a further specific subgroup of this embodiment, the crop protecting agent is a crop protecting agent with low water solubility and a melting point of not more than 110° C. or a mixture of such active ingredients. These include, for example, pyrachlostrobin (64° C.), prochloraz (47° C.), metrafenon (100° C.), alphacypermethrin (79° C.) and pendimethalin (58° C.).

In a further group of embodiments, the organic active of low molecular weight is an organic active suitable for cosmetic applications or an active mixture other than the aforementioned aromas. Preferred cosmetic actives for loading of the microparticles are especially active plant ingredients and plant extracts.

Examples of cosmetic actives are skin and hair pigmentation agents, tanning agents, bleaches, keratin-hardening substances, antimicrobial active ingredients, photofilter active ingredients, repellent active ingredients, hyperemic substances, keratolytic and keratoplastic substances, antidandruff active ingredients, antiphlogistics, keratinizing substances, antioxidative active ingredients and active ingredients acting as free-radical scavengers, skin moisturizing or humectant substances, regreasing active ingredients, deodorizing active ingredients, sebostatic active ingredients, plant extracts, antierythematous or antiallergic active ingredients and mixtures thereof.

Artificial tanning actives suitable for tanning the skin without natural or artificial irradiation with UV rays are, for example, dihydroxyacetone, alloxan and walnut shell extract. Suitable keratin-hardening substances are generally active ingredients as are also used in antiperspirants, for example potassium aluminum sulfate, aluminum hydroxychloride, aluminum lactate, etc. Antimicrobial active ingredients are used in order to destroy microorganisms and/or to inhibit their growth and thus serve both as preservatives and also as a deodorizing substance which reduces the formation or the intensity of body odor. These include, for example, customary preservatives known to the person skilled in the art, such as p-hydroxybenzoic esters, imidazolidinylurea, formaldehyde, sorbic acid, benzoic acid, salicylic acid, etc. Such deodorizing substances are, for example, zinc ricinoleate, triclosan, undecylenoic acid alkylolamides, triethyl citrate, chlorhexidine, etc. Suitable photofilter active ingredients are substances which absorb UV rays in the UV-B and/or UV-A region. Suitable UV filters are those mentioned above. Additionally suitable are p-aminobenzoic esters, cinnamic esters, benzophenones, camphor derivatives, and pigments which stop UV rays, such as titanium dioxide, talc and zinc oxide. Suitable repellent active ingredients are compounds capable of warding off or driving away certain animals, particularly insects, from humans. These include, for example, 2-ethyl-1,3-hexanediol, N,N-diethyl-m-toluamide, etc. Suitable hyperemic substances, which stimulate blood flowthrough the skin, are, for example, essential oils, such as dwarf pine, lavender, rosemary, juniperberry, roast chestnut extract, birch leaf extract, hayseed extract, ethyl acetate, camphor, menthol, peppermint oil, rosemary extract, eucalyptus oil, etc. Suitable keratolytic and keratoplastic substances are, for example, salicylic acid, calcium thioglycolate, thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiphlogistics, which counter skin irritations, are, for example, allantoin, bisabolol, Dragosantol, chamomile extract, panthenol, etc.

Further cosmetic actives are aspalatin, glycyrrhizin, caffeine, proanthocyanidin, hesperetin, rutin, luteolin, oleuropein, theobromine, bioflavonoids and polyphenols. Examples of plant extracts are also acai extract (Euterpe oleracea), acerola extract (Malpighia glabra), field horsetail extract (Equisetum arvense), agarius extract (Agarius blazei murill), aloe extract (Aloe vera, Aloe Barbadensis), apple extract (Malus), artichoke leaf extract (Cynara scolymus), artichoke blossom extract (Cynara edulis), arnica extract (Arnica Montana), oyster extract (Ostrea edulis), baldrian root extract (Valeriana officinalis), bearberry leaf extract (Arctostaphylos uva-ursi), bamboo extract (Bambus vulgaris), bitter melon extract (Momordica charantia), bitter orange extract (Citrus aurantium), nettle leaf extract (Urtica dioica), nettle root extract (Urtica dioica), broccoli extract (Brassica oleracea), watercress extract (Rorippa nasturtium), painted nettle extract (Coleus forskohlii), capsicum extract (Capsicum frutescens), extract from Centella asiatica (Gotu Kola), cinchona extract, cranberry extract (Vaccinium vitis-daea), turmeric extract (Curcuma longa), damiana extract (Tunera diffusa), dragonfruit extract (Pitahaya), extract from Echinacea purpurea, wheat placenta extract, edelweiss extract (Leotopodium alpinum), ivy extract (Hedera helix), bindii extract (Tribulus terrestris), Garcinia cambogia extract (Garcinia cambogia), ginkgo extract (Ginkgo biloba), ginseng extract (Panax ginseng), pomegranate extract (Punica granatum), grapefruit extract (Citrus paradisi), griffonia extract (Griffonia simplicifolia), green tea extract (Camellia sinensis), guarana extract (Paullinia cupana), cucumber extract (Cucumis sativus), dog rose extract (Rosa canina), blueberry extract (Vaccinium myrtillus), hibiscus extract (Malvacea), mallow extract, honey extracts, hops extract (Humulus), ginger extract (Zingiber officinale), Iceland moss extract (Cetraria islandica), jojoba extract (Simmondsia chinensis), St. John's Wort extract (Hypericum perforatum), coffee concentrate, cocoa bean extract (Theobroma cacao), cactus blossom extract, chamomile blossom extract (Matricaria recutita, Matricaria chamomilla), carrot extract (Daucus carota), kiwi extract (Aperygidae), kudzu extract (Pueraria lobata), coconut milk extract, pumpkinseed extract (Curcurbita pepo), cornflower extract (Centaurea cyanus), lotus flower extract, dandelion extract (Taraxacum officinale), maca extract (Lepidium peruvianum), magnolia blossom extract, mango extracts, milk thistle extract (Silybum marianum), marigold extract (Calendula officiennalis), mate extract (Hex paraguariensis), butcher's broom extract (Rugcus aculeatus), sea algae extracts, cranberry extracts (Vaccinium macrocarpon), Moringa Oleifera extract, extract from Moschus Malve (Malva moschata), evening primrose oil extract (Azadirachta indica), nettle extract (Urticaceae), olive leaf extract (Olea europea), orange extract (hesperidin), orchid extract, papaya extract (Carica papaya), peppermint extracts, extract from Carica papaya (Geissospermum), bitter orange extract (Citrus aurantioum), lingonberry extract (Vaccinium vitas-ideea), African cherry extract (Prunus africana), sugar beet extracts, resveratrole extract (Polygonum cuspidatum), rooibos extract (Aspalasthus Linnearis), rose blossom extract, horse chestnut extract (Aesculus hippocastanum), rosemary extract (Rosemarinus Officinalis), red clover extract (Trifolium platense), red wine extract (Vitis vinifera), saw palmetto extract (Serenoa repens), lettuce extract (Lactuca sativa), sandalwood extract (Santalum rubrum), sage extract (Salvia officinalis), horsetail extract (Equisetum), yarrow extract (Achillea millefolium), black pepper extract (Piper nigrum), black tea extract, waterlily extract (Nymphaea), white willow bark extract (Salix Alba), liquorice extract (Glycyrrhiza), devil's claw extract (Harpagophytum procumbens), thyme extract (Thymus vulgaris), tomato extract (Lycopersicum esculentum), grapeseed extract (Vitis vinifera), grapeskin extract (Vitis vinifera), watercress (Rorippa amphibia), willow bark extract (Salix alba), wormwood extract (Artemisia absinthium), white tea extract, yam root extract (Dioscorea opposita), yohimbe extract (Pausinystalia yohimbe), witch hazel extract (Hamamelis), cinnamon extract (Cinnamomum cassia Presl), lemon extract (Citrus) and onion extract (Allium cepa).

In a further group of embodiments, the organic active of low molecular weight is an organic active from construction chemistry. Preferred actives for loading of the microparticles for applications in construction chemistry are especially polymerization catalysts.

Useful polymerization catalysts include those suitable for curing of reactive resins, especially addition resins, condensation resins or oxidation-curing resins. For this purpose, the polymerization catalyst is a catalyst for a free-radical polymerization, a polycondensation and/or a polyaddition. The suitable catalysts for a free-radical polymerization especially include peroxide splitters, and the catalysts known from coatings technology for oxidatively drying oil and alkyd resins as driers or siccatives.

Suitable polycondensation catalysts are catalysts for silicone condensation and crosslinking. Polyaddition catalysts used may, for example, be catalysts for curing of epoxy resins. In addition, polyaddition catalysts used may, for example, be urethanization catalysts customarily used in polyurethane chemistry. These are compounds that accelerate the reaction of the reactive hydrogen atoms of isocyanate-reactive components with the organic polyisocyanates.

Useful polymerization catalysts especially include amines, phosphines and organic metal salts.

Amines useful as polymerization catalysts, especially for polyadditions, are especially tertiary amines such as triethylamine, tributylamine, N,N-dimethylcyclohexylamine (DMCHA), N-methyldicyclohexylamine, N,N-dimethylbenzylamine (BDMA), N-methyl-morpholine, N-ethylmorpholine, N-cyclohexylmorpholine, 2,2′-dimorpholinodiethyl ether (DMDEE), N,N,N′,N′-tetramethylethylenediamine, N,N,N′,N′-tetramethylbutylene-diamine, N,N,N′,N′-tetramethylhexylene-1,6-diamine, N,N,N′,N″,N′″-pentamethyl-diethylenetriamine (PMDETA), N,N,N′,N″,N′″-pentamethyldipropylenetriamine (PMDPTA), N,N,N-tris(3-dimethylaminopropyl)amine, bis(2-dimethylaminoethyl) ether (BDMAEE), bis(dimethylaminopropyl)urea, 2,4,6-tris(dimethylaminomethyl)phenol, and its salt with 2-ethylhexanoic acid and isomers thereof, 1,4-dimethylpiperazine (DMP), N-methylimidazole, 1,2-dimethylimidazole, 1-methyl-4-(2-dimethylaminoethyl) piperazine, 1-azabicyclo[3.3.0]octane, 1,4-diazabicyclo[2.2.2]octane (DABCO), 1,8-diazabicyclo[5.4.0]undec-7-ene (DBU), 1,5-diazabicyclo[4.3.0]non-7-ene (DBN).

Further useful polymerization catalysts, especially for polyadditions, include: tris(dialkylamino)-s-hexahydrotriazines, especially 1,3,5-tris(3-[dimethylamino]propyl) hexahydrotriazine.

It is also possible to use polymerization catalysts that are reactive towards isocyanates, especially for polyadditions. Besides at least one tertiary amino group, they comprise a primary or secondary amino group or a hydroxyl group. Examples thereof include N,N-dimethylaminopropylamine, bis(dimethylaminopropyl)amine, N,N-dimethylaminopropyl-N′-methylethanolamine, dimethylaminoethoxyethanol, bis(dimethylaminopropyl)amino-2-propanol, N,N-dimethylaminopropyldipropanolamine, N,N,N′-trimethyl-N′-hydroxyethyl bisaminoethyl ether, N,N-dimethylaminopropylurea, N-(2-hydroxy-propyl)imidazole, N-(2-hydroxyethyl)imidazole, N-(2-aminopropyl)imidazole and/or the reaction products of ethyl acetoacetate, polyether polyols and 1-(dimethylamino)-3-aminopropane that are described in EP-A 0 629 607.

Useful phosphines as polymerization catalysts, especially for polyadditions, are preferably tertiary phosphines, such as triphenylphosphine or methyldiphenyl-phosphine.

Organic metal salts useful as polymerization catalysts preferably have the general formula

LₘMⁿ⁺nA⁻

in which
the ligand L is an organic radical or an organic compound selected from alkyl, alkenyl, alkynyl, aryl, arylalkyl, alkylaryl, heteroaryl, heteroarylalkyl, alkyl heteroaryl and acyl, the ligand L having 1 to 20 carbon atoms, and the m ligands L being the same or different,
m is 0, 1, 2, 3, 4, 5 or 6,
M is a metal,
n is 1, 2, 3 or 4, and
the anion A⁻ is a carboxylate ion, alkoxylate ion or enolate ion.

The metal M is preferably selected from lithium, potassium, cesium, magnesium, calcium, strontium, barium, boron, aluminum, indium, tin, lead, bismuth, cerium, cobalt, iron, copper, lanthanum, manganese, mercury, scandium, titanium, zinc and zirconium; more particularly from lithium, potassium, cesium, tin, bismuth, titanium, zinc and zirconium.

The ligand L is preferably alkyl having 1 to 20 carbon atoms. More preferably L is alkyl having 1 to 10 carbon atoms, especially 1 to 4 carbon atoms, e.g. methyl, ethyl, n-propyl, isopropyl, n-butyl, isobutyl, sec-butyl or tert-butyl.

The carboxylate ion preferably has the formula R¹—COO⁻ where R¹ is selected from H, alkyl, alkenyl, alkynyl, aryl, arylalkyl, alkylaryl, heteroaryl, heteroarylalkyl, alkylheteroaryl and acyl, and where the R¹ radical has up to 20 carbon atoms, preferably 6 to 20 carbon atoms. Particularly preferred carboxylate ions are selected from the anions of natural and synthetic fatty acids, such as neodecanoate, isooctanoate and laurate, and the anions of resin acids and naphthenic acids.

The enolate ion preferably has the formula R²CH═CR³—O⁻ where R² and R³ are each selected from H, alkyl, alkenyl, alkynyl, aryl, arylalkyl, alkylaryl, heteroaryl, hetero-arylalkyl, alkylheteroaryl and acyl, and where the R² and R³ radicals each have up to carbon atoms. Specific examples are ethylacetonate, heptylacetonate or phenylacetonate. The enolate ion derives preferably from a 1,3-diketone having five to eight carbon atoms. Possible examples include acetylacetonate, the enolate of 2,4-hexanedione, the enolate of 3,5-heptanedione and the enolate of 3,5-octanedione.

The alkoxylate ion preferably has the formula R⁴—O⁻ where R⁴ is selected from alkyl, alkenyl, alkynyl, aryl, arylalkyl, alkylaryl, heteroaryl, heteroarylalkyl, alkylheteroaryl and acyl, and where the R⁴ radical has up to 20 carbon atoms.

In particular embodiments the organic metal compound is selected from
alkali metal carboxylates, such as lithium ethylhexanoate, lithium neodecanoate, potassium acetate, potassium ethylhexanoate, cesium ethylhexanoate;
alkaline earth metal carboxylates, such as calcium ethylhexanoate, calcium naphthenate, calcium octoate (available as Octa-Soligen® Calcium from OMG Borchers), magnesium stearate, strontium ethylhexanoate, barium ethyl-hexanoate, barium naphthenate, barium neodecanoate;
aluminum compounds, such as aluminum acetylacetonate, aluminum dionate (e.g. K KAT® 5218 from King Industries);
zinc compounds, for example zinc(II) diacetate, zinc(II) ethylhexanoate and zinc(II) octoate, zinc neodecanoate, zinc acetylacetonate;
tin compounds, such as tin(II) carboxylates, examples being tin(II) acetate, tin(II) octoate, tin(II) ethylhexanoate, tin(II) neodecanoate, tin(II) isononanoate, tin(II) laurate, and dialkyltin(IV) salts of organic carboxylic acids, examples being dimethyltin diacetate, dibutyltin diacetate, dibutyltin dibutyrate, dibutyltin bis(2-ethylhexanoate), dibutyltin dilaurate, dibutyltin maleate, dioctyltin dilaurate and dioctyltin diacetate, especially dibutyltin dilaurate;
titanium compounds, such as tetra(2-ethylhexyl) titanate;
zirconium compounds, such as zirconium ethylhexanoate, zirconium neodecanoate, zirconium acetylacetonate (e.g. K-KAT® 4205 from King Industries); zirconium dionates (e.g. K-KAT® XC-9213; XC-A209 and XC-6212 from King Industries); zirconium 2,2,6,6-tetramethyl-3,5-heptanedionate;
bismuth compounds, such as bismuth carboxylates, especially bismuth octoate, bismuth ethylhexanoate, bismuth neodecanoate or bismuth pivalate (e.g. K-KAT® 348, XC-B221, XC-C227, XC 8203 from King Industries, TIB KAT 716, 716LA, 716XLA, 718, 720, 789 from TIB Chemicals, and those from Shepherd Lausanne);
manganese salts, such as manganese neodecanoate, manganese naphthenate;
cobalt salts, such as cobalt neodecanoate, cobalt ethylhexanoate, cobalt naphthenate;
iron salts, such as iron ethylhexanoate;
mercury compounds, such as phenylmercury carboxylate.

Preferred organic metal compounds are dibutyltin dilaurate, dioctyltin dilaurate, zinc(II) diacetate, zinc(II) dioctoate, zirconium acetylacetonate and zirconium 2,2,6,6-tetramethyl-3,5-heptanedionate, bismuth neodecanoate, bismuth dioctoate and bismuth ethylhexanoate.

The present invention relates, in a first group A of embodiments, to a process for loading microparticles with at least one organic active by method (a).

The present invention relates, in a second group B of embodiments, to a process for loading microparticles with at least one organic active by method (b).

The present invention relates, in a third group C of embodiments, to a process for loading microparticles with at least one organic active by method (c).

The present invention relates, in a fourth group D of embodiments, to a process for loading microparticles with at least one organic active by method (d).

Among the abovementioned groups A, B, C and D, preference is given to groups A, B and D. Particular preference is given to groups A and D.

The present invention relates, in a fifth group AD of embodiments, to a process for loading microparticles with at least one organic active by method (a) in combination with step (d2) of method (d). In this embodiment, the liquid (d1) which is used for loading comprises, besides the active, at least one room temperature solid substance A in molten, emulsified, suspended or dissolved form in the liquid, and optionally one or more solvents. In these liquids, the active is typically in dissolved form, in emulsified form or in the form of a melt.

The present invention relates, in a sixth group BD of embodiments, to a process for loading microparticles with at least one organic active by method (b) in combination with step (d2) of method (d). In this embodiment, the liquid (d1) which is used for loading comprises, besides the active, at least one polymerizable substance B in emulsified or dissolved form in the liquid, and optionally one or more solvents. In these liquids, the active is typically in dissolved form, in emulsified form or in the form of a melt.

The present invention relates, in a seventh group CD of embodiments, to a process for loading microparticles with at least one organic active by method (c) in combination with step (d2) of method (d). In this embodiment, the liquid (d1) which is used for loading comprises, besides the active, at least one substance C which is in dissolved or molten form in the liquid and can be solidified by addition of polyvalent ions, and optionally one or more solvents. In these liquids, the active is typically in dissolved form, in emulsified form or in the form of a melt.

It will be apparent that a person skilled in the art will choose the respective embodiments such that the active on the one hand and the auxiliary A, B/C or D are compatible with one another, i.e. do not enter into any chemical reaction with one another in which the active is altered. For example, the person skilled in the art will generally not choose the group of embodiments B, BD, C and CD when the active is selected from the actives suitable for construction chemical applications, or within these groups of embodiments will select the constituents B and C such that they do not enter into any chemical reaction with the active under the conditions of filling or storage.

Among the abovementioned groups AD, BD and CD, preference is given to group AD.

In groups A, B, C and D, in the processes of the invention, the microparticles are impregnated with a liquid (1a), (1b), (1c) or (1d), i.e. treated in such a way that the cavity present in the unfilled microparticles is partly, particularly largely, filled by the liquid (1a), in general at least to an extent of 50%, especially at least to an extent of 70%, or completely, or some, particularly the majority, of the gas present in the microparticles is displaced by the liquid. The liquids comprise the active, and so, when treated with the liquid, the active penetrates through the pores into the cavities and hence into the interior of the microparticles. In the case of the embodiments of groups A, B and C, or in the case of the embodiments of groups AD, BD and CD, substance A, or B or C, present in the composition, penetrates simultaneously into the cavities and hence into the interior of the microparticles.

For impregnation of the microparticles with the respective liquid (1a), (1b), (1c) or (1d), the microparticles to be laden will generally be contacted with the respective liquid comprising the active, i.e. generally either (1a) or (1b) or (1c) or (1d). The contacting can in principle be effected in any desired manner, with the proviso that the contact time is sufficient that the liquid has at least wetted the microcapsules to be loaded and hence can penetrate via the pores into the cavity/cavities.

In one embodiment of the invention, the microparticles are impregnated by suspending the microparticles in the liquid comprising the active, in general one of the liquids (1a), (1b), (1c) or (1d).

In another embodiment of the invention, the microparticles are impregnated by a method whereby the liquid comprising the active, generally one of the liquids (1a), (1b), (1c) or (1d), is applied to the unladen microparticles in finely distributed form, especially in the form of droplets. For this purpose, the microparticles will naturally be used in solid form, especially in the form of a powder. More particularly, the unladen microparticles in powder form can be irrigated or sprayed with the respective liquid comprising the active. Surprisingly, the liquid droplets are rapidly absorbed by the unladen microparticles. Moreover, it is possible in this way to exactly dose the liquid used for impregnation and hence the aroma chemical, such that removal of excess liquid can be avoided, or the complexity associated therewith reduced.

The microparticles are typically impregnated with the respective liquid (1a), (1b), (1c) or (1d) at temperatures below the melting or softening point or range of the wall material, which can be determined by the person skilled in the art in a manner known per se, for example by means of differential scanning calorimetry (DSC). Typically, the treatment is effected at temperatures that are at least 5 K, especially at least 10 K, below the melting or softening point or range of the wall material, for example at not more than 80° C., particularly not more than 70° C., especially not more than 60° C., for example in the range from 0 to 80° C., particularly in the range from 10 to 70° C. and especially in the range from 20 to 60° C. The treatment time is typically in the range from 1 min to 10 h, particularly in the range from 5 min to 8 h and especially in the range from 0.5 to 6 h.

The active may be in liquid or molten form in the liquids (1a), (1b), (1c) or (1d), or in liquid or molten form in the simultaneous presence of a solvent, of a polymerizable substance B or of a substance C solidifiable by means of polyvalent ions in the liquid, in emulsified or suspended form, or preferably in dissolved form. In the liquid (1d), the active may also be present as such provided that it is liquid at the temperature at which the loading is effected, for example in the case of an active or active mixture that is liquid at room temperature (22° C.).

If the active is in liquid or molten form in the liquid, it may have entirely or partly melted. Preferably, the active has entirely melted and especially has a temperature of more than 5° C. above its melting point in the loading. If the active is in emulsified form in the liquid, it may either be the disperse phase or the continuous phase. If the active is in suspended form, it preferably has a particle size smaller than the average pore radius.

If the active is in dissolved form, it may have entirely or partly dissolved. The active has preferably entirely dissolved. The dissolution of the active can in principle be brought about by any components of the liquid in which the active is soluble, for example substances (A) or (B), a solvent or solvent mixture or an optional further active.

Alternatively, it is possible to use organic solvents or water or mixtures of water and water-miscible organic solvents. Suitable organic solvents are especially those in which the wall material is insoluble. Suitable organic solvents are especially C₁-C₄-alkanols such as methanol, ethanol, n-propanol, isopropanol, n-butanol, 2-butanol, tert-butanol and aliphatic and cycloaliphatic hydrocarbons such as n-pentane, n-hexane, hexane mixtures, n-heptane, cyclohexane, cycloheptane, methylcyclohexane, petroleum ether, white oils, diols such as propanediol and dipropylene glycol, C₈-C₂₂ fatty acid C₁-C₁₀-alkyl esters such as isopropyl myristate, di-C₆-C₁₀-alkyl ethers, e.g. dicapryl ether (Cetiol® OE from BASF SE), di-C₁-C₁₀-alkyl esters of aliphatic, aromatic or cyclo-aliphatic di- or tricarboxylic acids, for example dialkyl phthalates such as dimethyl and diethyl phthalate and mixtures thereof, dialkyl hexahydrophthalates, e.g. dimethyl cyclohexane-1,2-dicarboxylate, diethyl cyclohexane-1,2-dicarboxylate and diisononyl cyclohexane-1,2-dicarboxylate, and dialkyl adipates such as dibutyl adipate (e.g. Cetiol® B from BASF SE), C₈-C₂₂ fatty acid triglycerides, e.g. vegetable oils or cosmetic oils such as octanoyl/decanoyltriglyceride (e.g. the commercial product Myritol® 318 from BASF SE), and dimethyl sulfoxide and mixtures thereof.

The microparticles to be laden with the active may be used in the form of a powder or as a suspension in a solvent in which the wall material is insoluble. Preference is given to using the microparticles to be treated in the form of a powder. This can be suspended in the respective liquid (1a), (1b), (1c) or (1d) and is preferably applied dropwise or by spray application with the liquid. Alternatively, it is possible to mix a suspension of the microparticles to be laden with the liquid (1a), (1b), (1c) or (1d).

Examples of suitable apparatus for production of the suspension include magnetic stirrers, rollers, shakers, and various close-clearance stirrer units, e.g. anchor stirrers, helical stirrers. The duration of the mixing operation depends on the viscosity of the liquid at loading temperature and hence on the diffusion rate of the liquid into the microparticles, and is generally 5 minutes to 12 hours. Preferably, 1 part by weight of microparticles is suspended in 0.5 to 5 parts by weight, preferably 0.8 to 3 parts by weight, of the respective liquid (1a), (1b), (1c) or (1d). The suspension comprising the microparticles and the respective liquid (1a), (1b), (1c) or (1d) is generally kept at a temperature in the range from 0 to 80° C. for 1 minute to 10 hours. The suspension is preferably kept at a temperature in the range from 10 to 70° C. and especially in the range from 20 to 60° C. for 0.5 hour to 10 hours. Optionally, the microparticles are separated from the respective liquid (1a), (1b), (1c) or (1d) added in excess. The methods suitable for this purpose are, for example, filtration, centrifugation, decanting and drying, for example contact drying, fluidized bed drying, vacuum drying or spray drying.

Preferably, the unladen microparticles will be impregnated with a liquid comprising the active, for example one of the aforementioned liquids (1a), (1b), (1c) or (1d), by a method other than the suspending of the unladen microparticles in a liquid comprising the active. More particularly, for impregnation of the unladen microparticles, a liquid comprising the active, for example one of the aforementioned liquids (1a), (1b), (1c) or (1d), will be applied to the unladen microparticles in finely distributed form, especially in the form of droplets. For this purpose, the microparticles will be used in solid form, in particular in the form of a powder. In particular, the unladen microparticles as a powder will be subjected to dropwise application or spray application of the respective liquid which comprises the active, for example the abovementioned liquids (1a), (1b), (1c) or (1d).

In general, for this purpose, the unladen microparticles will be initially charged in solid form, in particular in the form of a powder, in a mixer for the mixing of solids with liquids and the desired liquid which comprises the active, for example one of the abovementioned liquids (1a), (1b), (1c) or (1d), will be added, preferably in finely divided form, especially in the form of droplets, for example in the form of discrete droplets or as a spray mist. In particular, the respective liquid which comprises the active, for example one of the abovementioned liquids (1a), (1b), (1c) or (1d), will be applied in finely divided form, especially in the form of droplets, to the microparticles to be laden that are in motion. For example, it is possible in a suitable manner to set the microparticles to be laden in motion, especially to create a fluidized bed of the microparticles to be laden, and to apply, for example by spraying or dropwise, the respective liquid which comprises the active, for example one of the abovementioned liquids (1a), (1b), (1c) or (1d), in finely divided form, to the microparticles which have been set in motion or are present in the fluidized bed. The spraying or dropwise application can take place in a manner known per se by means of one or more nozzles, for example by means of single-substance nozzles or two-substance nozzles, or by means of drop-formation devices. Suitable mixing apparatuses are dynamic mixers, especially forced mixers, or those with a mixer shaft, e.g. shovel mixers, paddle mixers or ploughshare mixers, but also free-fall mixers of this kind, e.g. drum mixers, and fluidized bed mixers. The duration of the mixing operation depends on the type of mixer and the viscosity of the liquid comprising the active at loading temperature and hence on the diffusion rate of the liquid into the microparticles. The time required for loading can be determined in a simple manner by the person skilled in the art. It is generally 1 minute to 5 hours, in particular 2 minutes to 2 hours or 5 minutes to 1 hour. Preferably, the respective liquid which comprises the active, for example one of the abovementioned liquids (1a), (1b), (1c) or (1d), is used in an amount of 0.2 to 5 parts by weight, preferably 0.5 to 4 parts by weight, based on 1 part by weight of the unladen microparticles. The spray application or dropwise application is generally at a temperature in the range from 0 to 80° C., particularly in the range from 10 to 70° C. and especially in the range from 20 to 60° C.

It may be advantageous to remove any residual water present from the microparticles. This can be effected, for example, by rinsing with ethanol or acetone, and/or blowing the microparticles dry with an inert gas such as air, nitrogen or argon. Optionally, for this purpose, predried and/or preheated inert gases may also be used. Preferably, the filled microparticles are subsequently washed, preferably with aqueous propanediol solution, for example as a 10% by weight solution.

Commonly known drying methods may be used for drying. For example, the particles may be dried by means of convective driers such as spray driers, fluidized bed driers, cyclone driers, contact driers such as pan driers, contact belt driers, vacuum drying cabinet or radiative driers such as infrared rotary tube driers and microwave mixing driers.

In the case of the first group of embodiments A and in the group of embodiments AD, the liquid (1a), in addition to the active and the optional solvent, comprises at least one nonpolymerizable substance A which is solid at room temperature.

The nonpolymerizable substance A is especially selected from
organic polymers, especially those that melt at a temperature in the range from 30 to 150° C.,
organic polymers solubilizable in any solvent present and especially in aqueous solvents,
waxes.

Examples of organic polymers that melt in the range from 30 to 150° C. are especially aliphatic polyamides, polyethylenes, to the extent that they are not among the waxes, polyalkylene glycols, polyisobutenes, aliphatic polyesters, polyvinyl acetates, poly-C₁-C₁₀-alkyl acrylates and aliphatic polyurethanes. Polymers of this kind preferably have a number-average molecular weight Mn in the range from 800 to 10 000 daltons.

The organic polymers solubilizable in any solvent present and especially in aqueous solvents especially include
water-soluble uncharged polymers;
polymers that are soluble in water at neutral or basic pH but are insoluble in water at a pH below pH 6;
polymers that are insoluble in water at neutral or basic pH but are soluble in water at a pH below pH 6.

All solubility data are based here on solubility at 25° C. and 1 bar. A polymer is considered to be soluble at the respective pH in water when it dissolves to an extent of at least 1 g/L.

Water-soluble uncharged polymers are, for example,
water-soluble synthetic polymers, e.g. poly-C₂-C₃-alkylene glycols such as polyethylene glycols, polypropylene glycols and polyethylene-co-polypropylene glycols, especially block copolymers (poloxamers), graft and comb polymers with poly-C₂-C₃-alkylene glycol side chains, such as polyethylene glycol, polypropylene glycol or polyethylene-co-polypropylene glycol side chains, e.g. copolymers of methyl methacrylate with methyl polyethylene glycol acrylate or methacrylate, graft copolymers of vinyl acetate with polyethylene glycol, and graft copolymers of vinyl acetate, N-vinylcaprolactam with polyethylene glycol, e.g. Soluplus® from BASF SE, polyvinyl alcohols and partly hydrolyzed polyvinyl acetates, polyvinylpyrrolidone, poly(vinylpyrrolidone-co-vinylcaprolactam), polyvinylcaprolactam graft copolymers,
uncharged modified celluloses such as hydroxypropyl methylcellulose (HPMC), hydroxypropyl cellulose (HPC) and methyl cellulose (MC);
uncharged water-soluble biopolymers, especially polysaccharide-based polymers such as alginates, pectins, gellans, carrageenans, and degraded starches, and also water-soluble proteins such as gelatin, zein and milk proteins.

The polymers that are soluble in water at neutral or basic pH but insoluble in water at a pH below pH 6 especially include polymers having acid groups, especially carboxylic acid groups, e.g.
synthetic polymers having carboxylic acid groups, such as copolymers of monoethylenically unsaturated carboxylic acids such as acrylic acid or methacrylic acid with C₁-C₄-alkyl (meth)acrylates, preferably in a molar ratio of monoethylenically unsaturated carboxylic acids to C₁-C₄-alkyl (meth)acrylate in the range from 5:1 to 1:2, especially in the range from 3:1 to 1:1.5, for example copolymers of methacrylic acid with ethyl acrylate (e.g. Eudragit® L 100-55, Kollicoat® MAE 100-55, Eudragit® L 30-D55, Kollicoat® MAE 30 DP), copolymers of methacrylic acid with methyl methacrylate (e.g. Eudragit® L 100 and Eudragit® S 100), terpolymers of methacrylic acid, methyl acrylate and methyl methacrylate (e.g. Eudragit® FS30 D or Eudraguard® biotic);
celluloses modified with carboxylic acid groups, such as hydroxypropyl methylcellulose acetate succinate (HPMCAS) and hydroxypropyl methylcellulose acetate phthalate (HPMCAP);
acidic biopolymers, especially acidic polysaccharide-based polymers such as gum arabic.

The polymers that are insoluble in water at neutral or basic pH but soluble in water at a pH below pH 6 especially include polymers having basic groups, especially amino groups, e.g.
synthetic polymers having amino groups, such as copolymers of monoethylenically unsaturated amines such as di-C₁-C₄-alkylamino-C₁-C₄-alkyl (meth)acrylates or di-C₁-C₄-alkylamino-C₁-C₄-alkyl(meth)acrylamides with C₁-C₄-alkyl (meth)acrylates, preferably in a molar ratio of monoethylenically unsaturated amine to C₁-C₄-alkyl (meth)acrylate in the range from 5:1 to 1:2, for example copolymers of 2-(dimethylamino)ethyl methacrylate with methyl methacrylate and/or butyl acrylate (e.g. Eudragit® E, e.g. Eudragit® E PO, or Eudraguard® Protect or Kollicoat® Smartseal).
basic biopolymers, especially basic polysaccharide-based polymers such as chitosan.

The waxes include, for example, plant waxes, animal waxes, mineral or fossil waxes, semisynthetic waxes and synthetic waxes.

Animal waxes are, for example, wool wax, china wax, beeswax and uropygial grease, but also tallow and other insect waxes.

Plant waxes are, for example, sugarcane wax, carnauba wax (carnauba wax palm), candelilla wax (from various Euphorbiaceae), cork wax, guaruma wax (Calathea lutea), ouricury wax (Syagrus coronata), cuba palm wax (Copernicia hospita), esparto wax (Lygeum spartum, Stipa tenacissima), cotton wax, rice bran wax, flax wax, peat wax and rose wax, jasmine wax or the petha wax from the wax gourd, and also myrtle wax (Myrica cerifera) and wax fig wax (Ficus variegata).

Examples of mineral or fossil waxes are mineral oil waxes, montan wax, ozokerite and paraffin wax.

Examples of semisynthetic waxes are chemically treated natural waxes, for example natural waxes modified by oxidation, hydrogenation, esterification or amidation, and also reaction products of wax acids with monohydric fatty alcohols or wax alcohols, amides of fatty acids and wax acids, reaction products of di- or triamines with vegetable or animal fatty acids, alcohol waxes and the like.

Examples of synthetic waxes are Fischer-Tropsch waxes, polyethylene waxes, oxidized polyethylene waxes, polyvinyl ether waxes, polypropylene waxes and the like.

Polyalkylene glycols include, for example, homopolymers and copolymers comprising a multitude of repeat units of the formula —(O—R¹)— where, in each case independently, R¹ is selected from groups of the CₙH₂ₙ forms where n is an integer in the range from 2 to 12, especially 2, 3 or 4, and which are referred to hereinafter as poly-C₂-C₄-alkylene glycols. Examples of poly-C₂-C₄-alkylene glycols are polyethylene glycols and polypropylene glycols and copolymers comprising polyethylene glycol and/or polypropylene glycol, especially block copolymers of polyethylene glycol with polypropylene glycol.

The nonpolymerizable substance A is preferably selected from waxes, especially plant waxes, animal waxes, polyalkylene glycols, especially poly-C₂-C₄-alkylene glycols, and mixtures thereof.

In a second variant, the nonpolymerizable substance A is selected from water-solubilizable polymers and waxes, and mixtures thereof.

Especially preferably, the nonpolymerizable substance A is selected from waxes, and very specifically from beeswax, carnauba wax and polyethylene glycol.

In the process of the first embodiment A, the mass ratio of the at least one active to the nonpolymerizable substance A will be preferably in the range from 10:90 to 99:1, further preferably in the range from 20:80 to 95:5.

The liquid (1a) consists preferably to an extent of at least 90% by weight, more preferably to an extent of at least 98% by weight, based on the liquid, of
i) the active,
ii) at least one nonpolymerizable substance A which is solid at room temperature,
iii) optionally one or more solvents.

In particular, the liquid (1a) consists of
i) 10% to 99% by weight, especially 20% to 95% by weight, based on the liquid, of the active,
ii) 1% to 90% by weight, especially 5% to 80% by weight, based on the liquid, of the at least one nonpolymerizable substance A which is solid at room temperature, and
iii) 0% to 80% by weight, especially 0% to 50% by weight, based on the liquid, of the one or more solvents.

If the nonpolymerizable substance A is an organic polymer that melts at a temperature in the range from 30 to 150° C., or is a wax, the liquid (1a) more preferably consists to an extent of at least 90% by weight, most preferably to an extent of at least 98% by weight, based on the liquid, of
i) the active, and the
ii) at least one nonpolymerizable substance A which is solid at room temperature,
   and especially to an extent of at least 98% by weight, based on the liquid, of
i) 10% to 99% by weight, especially 20% to 95% by weight, based on the liquid, of the active,
ii) 1% to 90% by weight, especially 5% to 80% by weight, based on the liquid, of the at least one nonpolymerizable substance A which is solid at room temperature.

If the nonpolymerizable substance A is a solubilizable organic polymer, the liquid (1a) more preferably consists to an extent of at least 90% by weight, most preferably to an extent of at least 98% by weight, based on the liquid, of
i) the active,
ii) at least one nonpolymerizable substance A which is solid at room temperature,
iii) and one or more solvents,
   and especially to an extent of at least 98% by weight, based on the liquid, of
i) 10% to 95% by weight, especially 20% to 90% by weight, based on the liquid, of the active,
ii) 1% to 80% by weight, especially 5% to 50% by weight, based on the liquid, of the at least one nonpolymerizable substance A which is solid at room temperature, and
iii) 10% to 89% by weight, especially 30% to 75% by weight, based on the liquid, of the one or more solvents.

The treatment of the microparticles with the liquid (1a) is effected in the manner described in general terms above, especially by spray application or dropwise application.

In a first variant of the groups of embodiments A, the liquid (1a) used is a melt consisting essentially of at least one active and at least one organic polymer and/or at least one wax, wherein the wax or organic polymer is in molten form or in the form of a solution in the active in the liquid. In that case, the treatment of the microparticles with the respective liquid (1a) is effected preferably at temperatures in the range from 20 to 80° C., particularly in the range from 30 to 70° C., particularly in the range from 35 to 65° C. and especially in the range from 40 to 60° C.

In a second variant of the groups of embodiments A, the liquid (1a) used is a mixture of an aqueous solution or emulsion of the water-solubilizable polymer and the active.

In the case of the second group of embodiments B and in the group of embodiments BD, the liquid (1b), in addition to the active and the optional solvent, comprises at least one polymerizable substance B.

The polymerizable substance B is preferably liquid at room temperature.

The polymerizable substance B is selected, for example, from ethylenically unsaturated monomers, silanes having hydroxyl or alkoxy groups, and oxidatively polymerizable aromatic compounds.

Examples of ethylenically unsaturated monomers are:
i) hydrophobic monoethylenically unsaturated monomers (monomers M1), e.g.:
   esters of monoethylenically unsaturated C₃-C₈ monocarboxylic acids with C₁-C₃₀-alkanols or C₅-C₈-cycloalkanols (monomers M1.1), especially esters of acrylic acid and/or of methacrylic acid with alkanols having 1 to 12 carbon atoms or cycloalkanols having 5 to 8 carbon atoms, such as methyl acrylate, methyl methacrylate, ethyl acrylate, ethyl methacrylate, n-butyl acrylate, n-butyl methacrylate, tert-butyl acrylate, tert-butyl methacrylate, n-hexyl acrylate, octyl acrylate, 2-ethylhexyl acrylate. 2-propylheptyl acrylate, cyclohexyl acrylate and cyclohexyl methacrylate;
   esters of monoethylenically unsaturated C₄-C₈ dicarboxylic acids with C₁-C₃₀-alkanols (monomers M1.2), such as the diesters of maleic acid and fumaric acid such as diethyl maleate and diethyl fumarate;
   cyanoacrylates (monomers M1.3), i.e. esters of 2-cyanopropenoic acid, for example with C₁-C₃₀-alkanols or C₅-C₈-cycloalkanols, e.g. methyl cyano-acrylate and ethyl cyanoacrylate;
   vinylaromatic hydrocarbons (monomers M1.4) such as styrene;
   butadiene or isoprene (monomers M1.5);
   olefins and haloolefins (monomers M1.6), such as ethylene, propene, vinyl chloride and vinylidene chloride;
   vinyl esters and allyl esters of saturated C₁-C₃₀ monocarboxylic acids (monomers M1.7), such as vinyl acetate, vinyl propionate, vinyl hexanoate, vinyl octanoate and vinyl esters of Versatic acids;
ii) hydrophilic monoethylenically unsaturated monomers (monomers M2), e.g.:
   monoethylenically unsaturated monocarboxylic acids having 3 to 8 carbon atoms (monomers M2.1), such as acrylic acid and methacrylic acid;
   monoethylenically unsaturated dicarboxylic acids having 4 to 8 carbon atoms (monomers M2.2), such as maleic acid, itaconic acid and citraconic acid;
   primary amides of monoethylenically unsaturated monocarboxylic acids having 3 to 8 carbon atoms (monomers M2.3), such as acrylamide and methacrylamide;
   N-vinyllactams (monomers M2.4) such as N-vinylpyrrolidone, N-vinylpiperidone, N-vinylmorpholinone and N-vinylcaprolactam;
   monoethylenically unsaturated monomers bearing urea or keto groups (monomers M2.5), such as 2-(2-oxoimidazolidin-1-yl)ethyl (meth)acrylate, 2-ureido(meth)acrylate, N-[2-(2-oxooxazolidin-3-yl)ethyl]methacrylate, acetoacetoxyethyl acrylate, acetoacetoxypropyl methacrylate, aceto-acetoxybutyl methacrylate, 2-(acetoacetoxy)ethyl methacrylate, diacetoneacrylamide (DAAM) and diacetonemethacrylamide;
   monoethylenically unsaturated sulfonic acids and salts thereof (monomers M2.6), such as vinylsulfonic acid, allylsulfonic acid, sulfoethyl acrylate, sulfoethyl methacrylate, sulfopropyl acrylate, sulfopropyl methacrylate, 2-hydroxy-3-acryloyloxypropylsulfonic acid, 2-hydroxy-3-methacryloyloxy-propylsulfonic acid, styrenesulfonic acids and 2-acrylamido-2-methyl-propanesulfonic acid, especially salts thereof, specifically the sodium salts thereof;
   monoethylenically unsaturated monomers having a phosphate or phosphonate group and salts thereof (monomers M2.7), such as vinylphosphonic acid, allylphosphonic acid, 2-phosphonoethyl acrylate, 2-phosphonoethyl methacrylate, phosphonopropyl acrylate, phosphono-propyl methacrylate, styrenephosphonic acids, 2-acrylamido-2-methyl-propanephosphonic acid, and phosphoric monoesters of the hydroxy-C₂-C₄-alkyl esters of monoethylenically unsaturated C₃-C₈ monocarboxylic acids specified hereinafter, for example the phosphoric monoesters of 2-hydroxyethyl (meth)acrylate, 3-hydroxypropyl (meth)acrylate, 4-hydroxy-butyl (meth)acrylate, and especially the salts of the aforementioned mono-ethylenically unsaturated monomers having a phosphate or phosphonate group, specifically the sodium salts thereof;
   hydroxy-C₂-C₄-alkyl esters of monoethylenically unsaturated C₃-C₈ monocarboxylic acids (monomers M2.8), especially hydroxy-C₂-C₄-alkyl esters of acrylic acid or of methacrylic acid, also referred to hereinafter as hydroxyalkyl (meth)acrylates, especially 2-hydroxyethyl (meth)acrylate, 3-hydroxy propyl (meth)acrylate, 4-hydroxybutyl (meth)acrylate, etc.;
      and
iii) polyethylenically unsaturated monomers having generally at least 2, 3 or 4 nonconjugated ethylenically unsaturated double bonds (monomers M3), e.g.:
   polyesters of ethylenically unsaturated monocarboxylic acids such as acrylic acid or methacrylic acid with polyhydroxyl compounds (monomers M3.1), especially aliphatic polyhydroxyl compounds, having 2 to 6 OH groups, e.g. diesters of acrylic acid with ethyleneglycol, propane-1,3-diol, propane-1,2-diol, butane-1,4-diol, hexane-1,6-diol, diethylene glycol, triethylene glycol, dipropylene glycol or tripropylene glycol; triesters of acrylic acid with trimethylolpropane or pentaerythritol and the tetraesters of acrylic acid with pentaerythritol, and the corresponding di-, tri- and tetraesters of methacrylic acid. Examples of suitable monomers are in particular trimethylolpropane diacrylate, trimethylolpropane triacrylate, ethylene glycol diacrylate, butanediol diacrylate, hexanediol diacrylate, dipropylene glycol diacrylate, tripropylene glycol diacrylate,
   di- and polyvinyl ethers and di- and polyallyl ethers of polyhydroxyl compounds (monomers M3.2), especially of aliphatic polyhydroxyl compounds, having 2 to 6 OH groups, such as butanediol divinyl ether, trimethylolpropane diallyl ether,
   aromatic divinyl compounds (monomers M3.3), such as divinylbenzene.

Preferred ethylenically unsaturated monomers are those from groups M1.1, M1.3, M2.1, M2.4 and M3.1 and mixtures thereof.

Silanes having hydroxyl or alkoxy groups are especially compounds of the following formula (I):

(R—O)ₖ—Si(R′)₃₋ₖ—[O—Si(R″)₂]ₘ—[O—Si(R′)₂₋ₚ(OR)ₚ]ₙ—O—R (I)

in which
k is 1, 2 or 3;
m is a number in the range from 0 to 50;
n is 0 or 1;
p is 0, 1 or 2;
R is H or C₁-C₄-alkyl and especially H, CH₃ or C₂H₅;
R′ is C₁-C₄-alkyl and especially CH₃;
R″ is C₁-C₄-alkyl and especially CH₃.

Among these, preference is given to those compounds in which k is 2 or 3 and m and n are 0, e.g. triethoxymethylsilane, trimethoxymethylsilane, tetraethyl silicate and tetramethyl silicate.

Oxidatively polymerizable aromatic compounds are those that polymerize on contact with oxygen. These especially include phenol and compounds having a phenol structure, e.g. phenol, hydroquinone, catechol and dopamine, and aromatic amines such as aniline and diaminobenzenes.

In general, the process of the invention is effected in such a way that the microparticles are treated in such a way that, after the filling of the cavities of the microparticles with the liquid (1b) or after the displacing of the gas present in the cavities of the microparticles by the liquid (1b), polymerization of substance B is brought about. Any solvent present in the liquid (1b) is removed. The solvent, if present, can be removed before, during or after the polymerization. The polymerization results, especially in the region of the pore orifices, in solidification of substance B and hence in effective sealing of the microparticles laden with the active.

The treatment of the microparticles with the liquid (1b) is effected in the manner described in general terms above. More particularly, the microparticles will be subjected to spray application or dropwise application of the liquid (1b).

The polymerization of substance B can be brought about in a manner known per se, for example
by a free-radical polymerization in the case of the ethylenically unsaturated compounds, e.g. by use of UV radiation, optionally in the presence of photoinitiators, or by use of polymerization initiators with a low decomposition temperature, e.g. by redox initiators;
by a moisture-triggered condensation in the case of the silanes having hydroxyl or alkoxy groups, and, in the case of the cyanoacrylates, for example, by use of acidic catalysts;
by an oxygen-triggered polymerization in the case of the oxidatively polymerizable substances.

The polymerization of substance B is preferably effected at temperatures below 80° C., particularly not more than 70° C., more preferably not more than 60° C. and especially not more than 50° C., for example in the range from 0 to 80° C., particularly in the range from to 70° C., more preferably in the range from 5 to 60° C. and especially in the range from to 50° C.

Examples of suitable photoinitiators are
alpha-hydroxyalkylphenones and alpha-dialkoxyacetophenones such as 1-hydroxycyclohexyl phenyl ketone, 2-hydroxy-2-methyl-1-phenyl-1-propanone, 2-hydroxy-1-{4-[4-(2-hydroxy-2-methylpropionyl)benzyl]phenyl}-2-methylpropan-1-one, 2-hydroxy-1-[4-(2-hydroxyethoxy)phenyl]-2-methyl-1-propanone or 2,2-dimethoxy-1-phenylethanone;
phenylglyoxalic esters such as methyl phenylglyoxalate;
benzophenones such as benzophenone, 2-hydroxybenzophenone, 3-hydroxybenzophenone, 4-hydroxybenzophenone, 2-methylbenzophenone, 3-methylbenzophenone, 4-methylbenzophenone, 2,4-dimethylbenzophenone, 3,4-dimethylbenzophenone, 2,5-dimethylbenzophenone, 4-benzoylbiphenyl, or 4-methoxybenzophenone;
benzil derivatives such as benzil, 4,4′-dimethylbenzil and benzil dimethyl ketal;
benzoins such as benzoin, benzoin ethyl ether, benzoin isopropyl ether and benzoin methyl ether;
acylphosphine oxides such as 2,4,6-trimethylbenzoyldiphenylphosphine oxide, ethoxy(phenyl)phosphoryl(2,4,6-trimethylphenyl)methanone and bis(2,4,6-trimethylbenzoyl)phenylphosphine oxide;
titanocenes such as the product sold under the Irgacure® 784 name by BASF SE;
oxime esters such as the product sold under the Irgacure® OXE01 and OXE02 name by BASF SE;
alpha-aminoalkylphenones such as
2-methyl-1-[4-(methylthio)phenyl-2-morpholinopropan-1-one,
2-(4-methylbenzyl)-2-dimethylamino-1-(4-morpholinophenyl)-1-butanone or
2-benzyl-2-dimethylamino-1-(4-morpholinophenyl)-1-butanone.

Preferred photoinitiators are selected, in particular, from the groups of the alpha-hydroxyalkylphenones, alpha-dialkoxyacetophenones, phenylglyoxalic esters, benzophenones, benzoins and acylphosphine oxides.

Photoinitiators are typically used in an amount of 0.1% to 5% by weight, based on the substances to be polymerized.

Polymerization initiators are typically used in an amount of 0.1% to 5% by weight, based on the substances to be polymerized.

Acidic catalysts are typically used in an amount of 0.01% to 3% by weight, based on the substances to be polymerized.

Examples of suitable polymerization initiators are peroxides and azo compounds, and what are called redox initiator systems. Suitable peroxides are both inorganic peroxides such as hydrogen peroxide or peroxodisulfates, e.g. mono- or dialkali metal or ammonium salts of peroxodisulfuric acid, e.g. the mono- or disodium salts thereof, the mono- or dipotassium salts thereof or the mono- or diammonium salts thereof, and also organic peroxides, such as alkyl hydroperoxides and aryl hydroperoxides, e.g. tert-butyl hydroperoxide, p-menthyl hydroperoxide or cumyl hydroperoxide, dialkyl or diaryl peroxides, e.g. di-tert-butyl ordicumyl peroxides. Examples of azo compounds are especially 2,2′-azobis(isobutyronitrile), 2,2′-azobis(2,4-dimethylvaleronitrile) and 2,2′-azobis(amidinopropyl)dihydrochloride (AIBA). Redox initiators typically comprise a reducing component and an oxidizing component. Examples of the latter are especially the aforementioned peroxides, especially hydrogen peroxide and tert-butyl hydro-peroxide. Reducing components are, for example, sulfur compounds in which the sulfur has an oxidation number below VI, for example alkali metal sulfites, e.g. potassium or sodium sulfite, alkali metal hydrogensulfites, e.g. potassium or sodium hydrogen sulfite, alkali metal bisulfites, e.g. potassium or sodium metabisulfite, formaldehyde-sulfoxylates, for example sodium or potassium formaldehydesulfoxylates, alkali metal salts, especially sodium and potassium salts of aliphatic sulfinic acids, enediols, such as dihydroxymaleic acid, benzoin, ascorbic acid and reducing sugars, e.g. sorbose, glucose, fructose ordihydroxyacetone. In place of the reducing agents, it is also possible to use salts of polyvalent transition metals, especially iron(II) salts such as iron(II) sulfate, iron(II) ammonium sulfate or iron(II) phosphates.

Suitable acidic catalysts are Brønsted acids and Lewis acids, especially aliphatic and aromatic carboxylic acids such as benzoic acid, formic acid, acetic acid, dichloroacetic acid, trifluoroacetic acid, organic sulfonic acids such as methanesulfonic acid, benzenesulfonic acid and toluenesulfonic acid, and also tin(IV) compounds such as dibutyltin dioctanoate, dibutyltin dilaurate and dibutyltin diethylhexanoate.

In the process of the second embodiment B and correspondingly in the embodiment BD, the mass ratio of the at least one active to the polymerizable substance B in the liquid (1b) will be preferably in the range from 10:90 to 99:1, further preferably in the range from 20:80 to 95:5.

The liquid (1b) consists preferably to an extent of at least 90% by weight, more preferably to an extent of at least 98% by weight, based on the liquid, of
i) the active,
ii) at least one polymerizable substance B,
iii) optionally a nonpolymerizable substance A which is solid at room temperature,
iv) optionally one or more solvents.

In particular, the liquid (1b) consists of
i) 10% to 99% by weight, especially 20% to 95% by weight, based on the liquid, of the active,
ii) 1% to 90% by weight, especially 5% to 80% by weight, based on the liquid, of the polymerizable substance B,
iii) 0% to 30% by weight, especially 0% to 10% by weight, based on the liquid, of the at least one nonpolymerizable substance A which is solid at room temperature, and
iv) 0% to 30% by weight, especially 0% to 10% by weight, based on the liquid, of the one or more solvents.

More preferably, the liquid (1b) consists to an extent of at least 90% by weight, most preferably to an extent of at least 98% by weight, based on the liquid, of
i) the active, and the
ii) at least one polymerizable substance B,
   and especially to an extent of at least 98% by weight, based on the liquid, of
i) 10% to 99% by weight, especially 20% to 95% by weight, based on the liquid, of the active,
ii) 1% to 90% by weight, especially 5% to 80% by weight, based on the liquid, of the at least one polymerizable substance B.

The liquid (1b) used is preferably an emulsion or solution consisting essentially of at least one active and at least one polymerizable substance B preferably selected from ethylenically unsaturated monomers, silanes having hydroxyl or alkoxy groups, and oxidative polymerizable aromatic compounds, and where the polymerizable substance B is in molten form or in the form of a solution in the active, or the active is in dissolved form in the polymerizable substance B.

The liquid (1b) may optionally comprise a nonpolymerizable substance A, where substance A is selected as described herein for the liquid (1a).

The treatment of the microparticles with the liquid (1b) is in principle effected in the manner described in general terms above. More particularly, the microparticles will be subjected to spray application or dropwise application in the liquid (1b).

The microparticles are typically treated with the liquid (1b) at temperatures below the polymerization temperature of the respective system, frequently at not more than 80° C., particularly not more than 60° C., more preferably not more than 50° C. and especially not more than 40° C., for example in the range from 0 to 80° C., particularly in the range from to 60° C., particularly in the range from 15 to 50° C. and especially in the range from to 40° C.

In the case of the third group of embodiments C and in the group of embodiments CD, the liquid (1c), in addition to the active and the optional solvent, comprises at least one substance C that can be solidified by addition of polyvalent ions.

The substance C is typically a polymer that bears a multitude of anionic or acidic groups, e.g. carboxyl groups or sulfo groups, that form insoluble salts or complexes on contact with polyvalent ions, for example Ca²⁺. Typical examples of polymers of this kind are polysaccharides that bear carboxyl groups or sulfo groups, e.g. alginates, pectins and carrageenans, which form chelates by contact with polyvalent ions, for example Ca²⁺, and are solidified. Further examples of substances C of this kind are water-soluble inorganic salts that form insoluble salts with polyvalent ions, for example Ca²⁺, e.g. alkali metal carbonates and ammonium carbonate.

The liquid (1c) may optionally additionally comprise a nonpolymerizable substance A, where substance A is selected as described herein for the liquid (1a).

In the liquid (1c), the mass ratio of the at least one active to substance C will be preferably in the range from 10:90 to 99:1, further preferably in the range from 20:80 to 95:5.

The liquid (1c) used is preferably a solution of substance C in a solvent that comprises the active in dissolved or emulsified form.

The liquid (1c) consists essentially, preferably to an extent of at least 90% by weight, more preferably to an extent of at least 98% by weight, based on the liquid, of
i) the active,
ii) at least one substance C,
iii) optionally a nonpolymerizable substance A which is solid at room temperature, as described above;
iv) optionally one or more solvents, especially at least one solvent for dissolution of substance C, especially when substance C is insoluble in the active.

In particular, the liquid (1c) consists of
i) 10% to 99% by weight, especially 20% to 95% by weight, based on the liquid, of the active,
ii) 1% to 95% by weight, especially 5% to 80% by weight, based on the liquid, of substance C,
iii) 0% to 30% by weight, especially 0% to 10% by weight, based on the liquid, of the at least one nonpolymerizable substance A which is solid at room temperature, and
iv) 0% to 80% by weight, especially 0% to 50% by weight, based on the liquid, of the one or more solvents.

More preferably, the liquid (1c) consists to an extent of at least 90% by weight, most preferably to an extent of at least 98% by weight, based on the liquid, of
i) the active, and the
ii) at least one substance C,
iv) and one or more solvents,
   and especially to an extent of at least 98% by weight, based on the liquid, of
i) 10% to 95% by weight, especially 20% to 90% by weight, based on the liquid, of the active,
ii) 1% to 80% by weight, especially 5% to 50% by weight, based on the liquid, of substance C, and
iv) 10% to 89% by weight, especially 30% to 75% by weight, based on the liquid, of the one or more solvents.

Useful solvents for the liquid C include especially water and aqueous mixtures that comprise, besides water, one or more water-miscible solvents. Suitable organic solvents are especially C₁-C₄-alkanols such as methanol, ethanol, n-propanol, isopropanol, n-butanol, 2-butanol, tert-butanol.

In general, the process for these groups C and CD of embodiments is effected in such a way that the microparticles are treated in such a way that, after the displacement of the gas by the liquid (1c) to give the resultant microparticles laden with the active and substance C, a solution of polyvalent ions is added in order to bring about precipitation of substance C and hence solidification thereof. Any solvent present will be removed in a manner known per se, for example under reduced pressure.

An alternative procedure is first to treat the microparticles with a solution comprising the polyvalent metal ions, especially calcium ions, optionally to remove solvent, so as to obtain solvent-free microparticles comprising the polyvalent ions, and then to conduct the treatment with the liquid (1c).

The treatment of the microparticles with the liquid (1c) is in principle effected in the manner described in general terms above. More particularly, the microparticles will be subjected to spray application or dropwise application in the liquid (1c).

The microparticles are treated with the liquid (1c) especially under the aforementioned temperature conditions. The treatment is preferably effected at temperatures of not more than 80° C., frequently not more than 70° C., preferably not more than 60° C., particularly not more than 50° C., for example in the range from 0 to 80° C., particularly in the range from 10 to 70° C., particularly in the range from 15 to 60° C. and especially in the range from 15 to 50° C.

For treatment of the microparticles with the solution of the polyvalent metal ions, the laden microparticles will generally be contacted with the solution. The contacting can in principle be effected in any desired manner, with the proviso that the contact time is sufficient that the solution has at least wetted the microcapsules and hence can penetrate into the pores. For this purpose, typically, the laden microparticles and the solution are mixed with one another, for example by subjecting the laden microparticles to spray or dropwise application of the solution, or especially by suspending the laden microparticles in the solution of the polyvalent metal ions.

Preferably, the solidification of substance C is effected at temperatures of not more than 80° C., frequently not more than 70° C., preferably not more than 60° C., particularly not more than 50° C., for example in the range from 0 to 80° C., particularly in the range from 10 to 70° C., particularly in the range from 15 to 60° C. and especially in the range from 15 to 50° C.

Subsequently, solidification of substance C will be brought about, for example by treating the microparticles that have been treated with the liquid (1c) with a solution of a salt of the polyvalent ions, especially an aqueous solution of a salt of the polyvalent ions. In this case, especially in the region of the pore orifices, there is solidification of substance C and hence effective sealing of the microparticles laden with the active. Suitable polyvalent ions are particularly Ca²⁺, Zn²⁺, Fe²⁺ and Fe³⁺, especially Ca²⁺. Suitable salts are particularly the halides, especially the chlorides, and the sulfates.

In the case of the fourth group (D) of embodiments, the microparticles are first impregnated with the liquid (1d) in order to introduce the active present therein into the cavity/cavities of the microparticles. The liquid (1d) comprises the active and optionally, especially when the active is solid at room temperature, additionally one or more solvents.

The liquid (1d) may optionally additionally comprise a nonpolymerizable substance A, where substance A is selected as described herein for the liquid (1a). The liquid (1d) may optionally additionally comprise a polymerizable substance B, where substance B is selected as described herein for the liquid (1b). The liquid (1d) may optionally additionally comprise a substance C, where substance C is selected as described herein for the liquid (1c). Preferably, the liquid (1d) comprises neither substance (B) nor substance (C). In particular, the liquid (1d) comprises neither substance (A) nor substance (B) or substance (C).

The liquid (1d) consists essentially, preferably to an extent of at least 90% by weight, more preferably to an extent of at least 98% by weight, based on the liquid, of
i) the active,
ii) optionally a nonpolymerizable substance A which is solid at room temperature,
iii) optionally a polymerizable substance B, and
iv) optionally one or more solvents.

In a specific embodiment, the liquid (1d) consists, especially to an extent of at least 90% by weight, more preferably to an extent of at least 98% by weight, based on the liquid, of the active which is preferably an active which is liquid at room temperature, i.e. at 22° C. and 1016 mbar.

In a further specific embodiment, the liquid (1d) consists to an extent of at least 90% by weight, more preferably to an extent of at least 98% by weight, based on the liquid, of
i) 30% to 95% by weight and especially 50% to 90% by weight, based on the liquid, of the active, and
iv) 30% to 95% by weight and especially 50% to 90% by weight, based on the liquid, of one or more solvents.

Suitable organic solvents for the liquid (1d) are especially those in which the wall material is insoluble. Suitable organic solvents are especially C₁-C₄-alkanols such as methanol, ethanol, n-propanol, isopropanol, n-butanol, 2-butanol, tert-butanol and aliphatic and cycloaliphatic hydrocarbons such as n-pentane, n-hexane, hexane mixtures, n-heptane, cyclohexane, cycloheptane, methylcyclohexane, petroleum ether, white oils, diols such as propanediol and dipropylene glycol, C₈-C₂₂ fatty acid C₁-C₁₀-alkyl esters such as isopropyl myristate, di-C₆-C₁₀-alkyl ethers, e.g. dicapryl ether (Cetiol® OE from BASF SE), di-C₁-C₁₀-alkyl esters of aliphatic, aromatic or cycloaliphatic di- or tricarboxylic acids, for example dialkyl phthalates such as dimethyl and diethyl phthalate and mixtures thereof, dialkyl hexahydrophthalates, e.g. dimethyl cyclohexane-1,2-dicarboxylate, diethyl cyclohexane-1,2-dicarboxylate and diisononyl cyclohexane-1,2-dicarboxylate, and dialkyl adipates such as dibutyl adipate (e.g. Cetiol® B from BASF SE), C₈-C₂₂ fatty acid triglycerides, e.g. vegetable oils or cosmetic oils such as octanoyl/decanoyltriglyceride (e.g. the commercial product Myritol® 318 from BASF SE), and dimethyl sulfoxide and mixtures thereof.

The treatment of the microparticles with the liquid (1d) is effected in the manner described in general terms above. More particularly, the microparticles will be subjected to spray application or dropwise application in the liquid (1d).

The microparticles are treated with the liquid (1d) especially under the aforementioned temperature conditions. The treatment is preferably effected at temperatures of not more than 80° C., frequently not more than 70° C., preferably not more than 60° C., particularly not more than 50° C., for example in the range from 0 to 80° C., particularly in the range from 10 to 70° C., particularly in the range from 15 to 60° C. and especially in the range from 15 to 50° C.

Subsequently, the microparticles are sealed by applying a substance that seals the pores of the laden microparticles to the surface of the microparticles that have been laden with the active.

For this purpose, the procedure will preferably be to apply a solid coating to the surface of the microparticles that have been laden with the active. This coating leads to sealing of the pores.

The procedure will preferably be such that this solid coating has an average layer thickness in the range from 0.005 to 0.75 times, especially 0.025 to 0.65 times, the average diameter, i.e. the D[4,3] value, of the untreated microparticles, or 0.01 to 1.5 times, especially 0.05 to 1.3 times, the average radius of the untreated microparticles. The average layer thickness is typically in the range from 50 nm to 25 μm and preferably in the range from 80 nm to 8 μm. For microparticles having a D[4,3] value in the range from 100 to 400 μm, the average layer thickness is preferably in the range from 0.5 μm to 25 μm. For microparticles having a D[4,3] value in the range from 2 to 50 μm, especially 5 to 30 μm, the average layer thickness is preferably in the range from 50 nm to 1 μm.

However, it is possible in principle to apply a substance to the surface of the microparticles that have been laden with the active in such a way that only the pores of the laden microparticles are sealed. This is possible, for example, in the case of laden microparticles that have been impregnated with the liquid (1b) or (1c). In this case, for example, in step (d2), a substance that brings about polymerization of the polymerizable substance B can be applied to the surface of the laden microparticles, or a solution comprising polyvalent ions that bring about the solidification of substance C. In the case of such a procedure, the polymerization/solidification will generally take place in the region of the pore orifices and thus bring about effective sealing of the pores.

In a preferred group of embodiments of the invention, a solid coating will be applied to the surface of the microparticles that have been laden with the active by treating the microparticles with a liquid (2d) comprising a film-forming substance D so as to form a solid layer on the surface of the microparticles.

In particular, the liquid (2d) consists essentially, preferably to an extent of at least 90% by weight, more preferably to an extent of at least 98% by weight, based on the liquid, of
i) at least one film-forming substance D,
ii) optionally one or more solvents.

More preferably, the liquid (2d) consists to an extent of at least 90% by weight, more preferably to an extent of at least 98% by weight, based on the liquid, of
i) 10% to 100% by weight, based on the liquid, of at least one film-forming substance D in molten, emulsified, dispersed or dissolved form in the liquid, and
ii) 0% to 90% by weight, based on the liquid, of one or more solvents.

In a specific embodiment, the liquid (2d) consists especially to an extent of at least 90% by weight, more preferably to an extent of at least 98% by weight, based on the liquid, of the film-forming substance D.

In a further specific embodiment, the liquid (2d) consists to an extent of at least 90% by weight, more preferably to an extent of at least 98% by weight, based on the liquid, of
i) 30% to 95% by weight and especially 50% to 90% by weight, based on the liquid, of the film-forming substance D, and
iv) 30% to 95% by weight and especially 50% to 90% by weight, based on the liquid, of one or more solvents.

Suitable solvents for the liquid (2d) are those in which the film-forming substance D is soluble or solubilizable, dispersible or emulsifiable. Moreover, solvents for the liquid should be restricted to those solvents in which the wall material is insoluble. These include water and organic solvents in which the wall material is insoluble, and mixtures thereof. Suitable organic solvents are especially C₁-C₄-alkanols such as methanol, ethanol, n-propanol, isopropanol, n-butanol, 2-butanol, tert-butanol and aliphatic and cycloaliphatic hydrocarbons such as n-pentane, n-hexane, hexane mixtures, n-heptane, cyclohexane, cycloheptane, methylcyclohexane, petroleum ether, white oils, diols such as propanediol and dipropylene glycol, C₈-C₂₂ fatty acid C₁-C₁₀-alkyl esters such as isopropyl myristate, di-C₆-C₁₀-alkyl ethers, e.g. dicapryl ether (Cetiol® OE from BASF SE), di-C₁-C₁₀-ALKYL esters of aliphatic, aromatic or cycloaliphatic di- or tricarboxylic acids, for example dialkyl phthalates such as dimethyl and diethyl phthalate and mixtures thereof, dialkyl hexahydrophthalates, e.g. dimethyl cyclohexane-1,2-dicarboxylate, diethyl cyclohexane-1,2-dicarboxylate and diisononyl cyclohexane-1,2-dicarboxylate, and dialkyl adipates such as dibutyl adipate (e.g. Cetiol® B from BASF SE), and mixtures thereof.

According to the nature of the film-forming substance D, it may be in dissolved, dispersed or liquid/molten form in the liquid (2d). Suitable film-forming substances D are, for example, the aforementioned substances A, B and C.

In a preferred group of embodiments, the film-forming substance D is selected from the aforementioned nonpolymerizable substances A, especially from
the aforementioned organic polymers that melt at a temperature in the range from 30 to 150° C.,
the aforementioned organic polymers solubilizable in any solvent present and especially in aqueous solvents,
organic polymers dispersible in any solvent present and especially in aqueous solvents, and
the waxes.

In this group of embodiments, the film-forming substance D is preferably selected from
waxes, especially from plant waxes or animal waxes,
polyalkylene glycols, especially poly-C₂-C₄-alkylene glycols,
water-insoluble organic polymers that melt at a temperature in the range from 30 to 150° C., and mixtures thereof.

In this case, the film-forming substance D is especially used as a solution in a suitable solvent or as a melt.

In a second variant, the film-forming substance D is selected from polymers that are solubilizable or dispersible in water. In this case, substance D is especially used as a solution, emulsion or dispersion in water.

The polymers that are solubilizable in aqueous solvents and are suitable as film-forming substances D especially include the following that have been mentioned as constituent A:
water-soluble uncharged polymers;
polymers that are soluble in water at neutral or basic pH but are insoluble in water at a pH below pH 6; and
polymers that are insoluble in water at neutral or basic pH but are soluble in water at a pH below pH 6.

The polymers that are dispersible in aqueous solvents and are suitable as film-forming substances D include
vinyl acetate homopolymers and copolymers, such as polyvinyl acetate (e.g. Collicoat® SR30 D from BASF SE), vinyl acetate-ethylene copolymers, vinyl acetate-vinylpyrrolidone copolymers, vinyl acetate-vinylcaprolactam copolymers, methyl methacrylate-diethylaminoethyl methacrylate copolymers, and which are especially used in the form of an aqueous polymer dispersion;
water-dispersible waxes that are typically used in the form of aqueous wax emulsions or dispersions;
water-dispersible or -swellable polysaccharides such as starch or cellulose, including modified starches and water-insoluble cellulose derivatives, e.g. hydrophobically modified starches, e.g. ethyl cellulose and chitin, and which are especially used in the form of an aqueous suspension;
proteins, to the extent that they are insoluble in water, for example denatured proteins, e.g. milk proteins such as casein and whey protein, wheat protein, egg protein, soya protein, peanut protein and keratins, and also coacervates of proteins such as gelatin A+gelatin B, gelatin B+gum arabic, gelatin A+pectin, casein+gum arabic.

More preferably, the film-forming substance D is selected from plant or animal waxes, polyalkylene glycols, polyvinyl acetate and mixtures thereof that are specified herein. In that case, it is specifically a wax, especially beeswax or carnauba wax.

The film-forming substance D may also be selected, for example, from the afore-mentioned polymerizable substances B, in which case the film formation comprises polymerization of substance B. In this case, the polymerizable substance B is preferably selected from the aforementioned ethylenically unsaturated monomers, the silanes having hydroxyl or alkoxy groups, especially those of the general formula (I), and oxidatively polymerizable aromatic compounds. In this case, the liquid (2d) consists particularly to an extent of at least 90% by weight, more preferably to an extent of at least 98% by weight, based on the liquid, of substance B, and optionally comprises a substance that brings about the polymerization, e.g. a photoinitiator or a polymerization initiator.

The film-forming substance D may also be selected from substances C that can be solidified by addition of polyvalent metal ions, in which case the film formation requires the presence of polyvalent metal ions. In this case, the substance is preferably selected from alginates, pectins and carrageenans.

Optionally, the microparticles are separated from the liquid (1d) added in excess prior to the treatment with the liquid (2d). The methods suitable for this purpose are, for example, filtration, centrifugation, decanting, and drying, for example by means of convective driers such as spray driers, fluidized bed driers, cyclone driers, contact driers such as pan driers, contact belt driers, vacuum drying cabinet or radiative driers such as infrared rotary tube driers and microwave mixing driers.

Typically, the application rate of substance D and the manner of treatment in step (d2) are chosen such that this solid layer covers the entire surface of the respective microparticle. In particular, the application rate of substance D and the manner of treatment are chosen such that this solid layer has an average layer thickness in the range from 0.005 to 0.75 times, especially 0.025 to 0.65 times, the average diameter, i.e. the D[4,3] value, of the microparticles, or 0.01 to 1.5 times, especially 0.05 to 1.3 times, the average radius of the microparticles. The average layer thickness is typically in the range from 50 nm to 25 μm and preferably in the range from 80 nm to 8 μm. For microparticles having a D[4,3] value in the range from 100 to 600 μm, especially having a D[4,3] value in the range from 100 to 400 μm, the average layer thickness is preferably in the range from 0.5 μm to 25 μm. For microparticles having a D[4,3] value in the range from 2 to 50 μm, especially having a D[4,3] value in the range from 5 to 30 μm, the average layer thickness is preferably in the range from 50 nm to 1 μm. Preferably, the application rate of substance D and the manner of treatment are chosen such that the resultant mass ratio of the laden microparticles used to the film-forming substance D on the particles is in the range from 95:5 to 20:80 and especially in the range from 90:10 to 50:50.

For treatment of the microparticles with the liquid (2d), the laden microparticles will generally be contacted with the liquid (2d). The contacting can in principle be effected in any desired manner, with the proviso that the contact time is sufficient for the liquid (2d) to have at least wetted the microcapsules. For this purpose, the laden microparticles and the liquid (2d) are typically mixed with one another, for example by subjecting the laden microparticles to spray application or dropwise application of the liquid (2d).

In general, for this purpose, the laden microparticles will be initially charged in a mixer for the mixing of solids with liquids and the desired liquid (2d) will be added, preferably in finely divided form, for example in the form of droplets or as a spray mist. In particular, the liquid (2d) will be applied to the laden microparticles that are in motion. For example, it is possible in a suitable manner to set the microparticles in motion mechanically, for example by agitating or with the aid of moving mixing elements, or to create a fluidized bed of the laden microparticles and to apply, for example by spraying or dropwise, the liquid (2d) in finely divided form, especially in droplet form, to the microparticles thus set in motion, for example to the microparticles present in the fluidized bed. The fluidized bed can be generated mechanically, for example by rotating mixing elements or by means of a gas stream. Suitable mixing apparatuses are dynamic mixers, especially forced mixers, or those with a mixer shaft, e.g. shovel mixers, paddle mixers or ploughshare mixers, but also free-fall mixers of this kind, e.g. drum mixers, and fluidized bed mixers.

The microparticles used in step (d2) may be used in the form of a suspension of microparticles as obtained from step (d1). The microparticles used in step (d2) may also be used in the form of a powder. In that case, the powder is preferably obtained by drying the microparticles obtained in step (d1). In this case, the microparticles, prior to drying, can also be rinsed and/or washed and/or transferred into a further liquid medium and/or contacted therewith.

In a preferred group of embodiments, the untreated microparticles are first impregnated with the liquid (1d) in step (d1), then optionally dried, and immediately thereafter treated with the liquid (2d). In particular, the microparticles will be subjected to spray application or dropwise application of the liquid (1d) in step (d1) and the laden microparticles thus obtained will be subjected to spray application or dropwise application of the liquid (2d) immediately thereafter, preferably in the same apparatus.

In a first group of embodiments, the microparticles laden with the active will be treated with a melt or a solution of the nonpolymerizable substance A or a dispersion, especially an aqueous dispersion, of the water-insoluble film-forming polymer, for example by suspending the microparticles laden with the active that have been produced in step (d1) in the solution or the melt, or especially by subjecting the microparticles laden with the active that have been produced in step (d1) to dropwise application or spray application of the solution, dispersion or melt. On cooling of the melt or on removal of the solvent, the layer of the nonpolymerizable substance A or of the film-forming polymer forms on the microparticles.

In a second group of embodiments, the microparticles laden with the active will be treated with a melt or a solution of the polymerizable substance B, for example by suspending the microparticles laden with the active that have been produced in step (d1) in the solution or the melt or especially by subjecting the microparticles laden with the active that have been produced in step (d1) to dropwise application or spray application of the solution or the melt. Subsequently, the polymerization of substance B is triggered, for example by, in the case of a photopolymerizable liquid (2d), irradiating the microparticles treated therewith with UV light of the suitable wavelength, or, in the case of a conventionally polymerizable composition, subjecting the microparticles treated with the liquid (2d) to the polymerization conditions necessary for the purpose, for example ingress of moisture or oxygen or heating to polymerization temperature.

In a further group of embodiments, the microparticles laden with the active will be treated with the liquid (2d) comprising substance C, i.e. with a melt or a solution of substance C, for example by suspending the microparticles laden with the active that have been produced in step (d1) in the solution or melt of substance C or especially by subjecting the microparticles laden with the active that have been produced in step (d1) to dropwise application or spray application of the solution or the melt of substance C. Subsequently, solidification of substance C will be brought about, for example by treating the microparticles that have been treated with the liquid (2d) with a solution of a salt of the polyvalent ions, especially an aqueous solution of a salt of the polyvalent ions. The treatment with the solution of the polyvalent ions is typically effected by spraying of the microparticles treated with the liquid (2d), or by suspending the microparticles treated with the liquid (2d) in a solution of the polyvalent ions. Suitable polyvalent ions are particularly Ca²⁺, Zn²⁺, Fe²⁺ and Fe³⁺, especially Ca²⁺. Suitable salts are particularly the halides, especially the chlorides, and the sulfates.

In a further group of embodiments of the invention, a solid coating will be applied to the surface of the microparticles laden with the active, by powdering the microparticles with a finely divided solid and then bringing about formation of a film of the finely divided solid.

Suitable particulate substances especially include gel-forming organic materials, for example polysaccharides such as starch, starch derivatives, cellulose, cellulose derivatives, the aforementioned biodegradable polyesters, especially the aforementioned aliphatic or semiaromatic polyesters, e.g. PBAT and PBSeT, and gel-forming inorganic materials, for example fumed silica, precipitated silica or phyllosilicates, especially clay minerals which may also be used in the form of what are called nanosilicates.

Typically, the particulate substances used for powdering have average particle sizes much smaller than the average diameter of the microparticles. In particular, finely divided solids having an average diameter, i.e. D[4,3] value, in the range from 0.001 to 0.2 times the D[4,3] value of the microparticles will be used. For microparticles having a D[4,3] value in the range from 100 to 600 μm, especially having a D[4,3] value in the range from 100 to 400 μm, finely divided solids having a D[4,3] value in the range from 0.5 μm to 20 μm will preferably be used. For microparticles having a D[4,3] value in the range from 2 to 50 μm, especially having a D[4,3] value in the range from 5 to 30 μm, finely divided solids having a D[4,3] value in the range from 50 nm to 1 μm will preferably be used.

In general, for this purpose, the laden microparticles will be initially charged in a mixer for the mixing of solids and the desired finely divided solid will be added, for example by means of a gas stream. In particular, the finely divided solid will be applied to the laden microparticles that are in motion. For example, it is possible in a suitable manner to create a fluidized bed of the laden microparticles and to apply the desired finely divided solid to the microparticles present in the fluidized bed. Suitable mixing apparatuses are dynamic mixers, especially forced mixers, or those with a mixer shaft, e.g. shovel mixers, paddle mixers or ploughshare mixers, but also free-fall mixers of this kind, e.g. drum mixers, and fluidized bed mixers.

The formation of a film of the finely divided solid can be brought about, for example, by applying a substance that brings about swelling or crosslinking of the finely divided solid and hence film formation in the manner described for liquid (2d). In the case of the aforementioned water-swellable finely divided solids, the film formation can be effected in a simple manner by spraying with water. Alternatively, it is possible to apply one of the above-described liquids (2d) to the surface of the microparticles powdered with the finely divided solid, and hence to bring about film formation on the surface.

In a further group of embodiments of the invention, a solid coating will be applied to the surface of the microparticles laden with the active by depositing a volatile substance from the gas phase on the surface of the microparticles and converting it to a solid from the surface by chemical reaction. The applying of solid coatings to surfaces by deposition of volatile substances from the gas phase with chemical conversion of the volatile substance to a solid can be effected in analogy to processes known per se, for example by chemical vapor deposition (CVD) or the related technique of atomic layer deposition (ALD).

In the case of CVD and likewise in the case of ALD, the creation of the coating is implemented via a chemical reaction of at least one volatile precursor substance with a further reactive substance, the co-reagent, on the surface of the material to be coated, the microparticles here. In conventional CVD, the co-reagent and the precursor are in the same gas phase. By contrast with conventional CVD methods, in ALD, the starting materials are admitted into the reaction chamber in cyclical succession. Between the admissions of the starting material gases, the reaction chamber is normally purged with an inert gas (e.g. argon). In this way, the component reactions can be clearly separated from one another and restricted to the surface.

In general, the precursor is selected such that reaction thereof with the co-reagent gives rise to an inorganic solid, for example an oxide, hydroxide, hydride, carbide or nitride of a metal or semimetal, or forms a metal or semimetal in elemental form on the surface of the microparticles. Typical coatings that can be deposited by means of methods of this kind consist essentially of Al₂O₃, SiO₂, TiO₂, ZrO₂, HfO₂, Ta₂O₃, WO₃, tungsten carbide, titanium carbide or silicon nitride.

Examples of precursors are metal alkyl compounds such as trimethylaluminum, amino metal compounds, such as tetrakis(dimethylamino)titanium (TDMAT), pentakis(dimethylamino)tantalum (PDMAT) and tetrakis(dimethylamido)zirconium (TDMAZ), (semi)metal alkoxides, such as tetramethyl orthosilicate, tetraethyl orthosilicate (TEOS) and zirconium tert-butoxide, (semi)metal halides such as SnCl₄, SiH₂Cl₂, SiHCl₃, HfCl₄, WF₆, TaCl₅ or TiCl₄, and metal hydrides.

Typically, a reaction is caused on the surface of the particles by a co-reagent that brings about the conversion of the precursor to the solid coating. The nature of the co-reagent is guided in a manner known per se by the precursor and the desired coating. Examples of co-reagents are H₂O, O₂, O₃, formic acid and hydrogen.

Typically, the conversion is effected at temperatures in the range from 25 to 100° C. The conversion can be promoted by plasma excitation. Methods of this kind are familiar to those skilled in the art. A review of methods of this kind can be found in K. L. Choy, Progress in Materials Science, 48 (2003), pp. 57-170.

The microparticles thus obtained comprise the active in a form bound by any polymerized substances A, B and C or in a sealed form of the microparticles via application of a further substance. Therefore, the microparticles laden with the active can be stored over a prolonged period without any loss of active. Moreover, the choice of the respective substance A, B, C or D enables control of the release characteristics of the active from the microparticles and hence the controlled release thereof.

For example, in the case of the laden microparticles produced in embodiments A, B and C, the active is released continuously to the surrounding medium over a prolonged period, and the release of the active generally proceeds to an enhanced degree at higher temperatures and can therefore be promoted by heating. If a water-solubilizable polymer is used in embodiment A, the release can also be promoted by contacting the microparticles with water or an aqueous solution, optionally at a pH at which the water-solubilizable polymer is soluble.

If a protein is used in embodiment A, this can also be enzymatically degraded by proteases.

In the embodiments of the invention in which the particles have been treated with a liquid d2, i.e. in groups D, AD, BD and CD of embodiments, the release can be promoted by damaging and/or partly or completely removing the coating created in step (d2) or the sealing of the pores. This is accomplished, for example, by the following measures:
1) Mechanical stress on the laden microparticles, for example by friction. This damages and generally partly removes the coating;
2) By enzymatic degradation, i.e. by treatment of the microparticles with enzymes, for example with proteases when the coating or pore seal comprises a protein, with cellulases when the coating or pore seal comprises a cellulose or cellulose derivative, or with esterases when the coating or pore seal and/or the wall material comprises polyester;
3) By contacting with water or an aqueous solution with a suitable pH when the coating or the pore seal is a water-soluble polymer or a polymer which is soluble in water above a particular pH or below a particular pH. It is possible here to achieve, for example, destruction of the layer of the polymer and hence the release in a controlled manner by altering the pH;
4) Thermal treatment, for example when substance D comprises a low-melting polymer or a polymer which is destroyed on heating;
5) Exposure to light, especially UV light, when the coating is UV-labile;
6) By ambient conditions as occur on use of the microparticle compositions or on use of formulations comprising the microparticle compositions, for example on contact with body fluids such as perspiration or urine, by exposure to light or by contact with microorganisms; and
7) Combinations of the aforementioned measures.

Measure 2) is of interest especially when the laden microparticle compositions of the invention are to be used in enzyme-containing formulations, for example in washing or cleaning products where the enzyme action does not occur until under use conditions. In this way, the active, e.g. an aroma, can be released in a specific and hence controlled manner in use.

Measure 3) is of interest particularly when the laden microparticle compositions of the invention are formulated in compositions that come into contact with aqueous liquids in use, for example in crop protection products or pharmaceutical administration forms. In the latter, for example, in the case of oral administration, release can be achieved in a controlled manner in the stomach or in the intestine when the layer of the film-forming substance D comprises a polymer which is soluble either at acidic pH values or at basic pH values.

Measure 4) can be effected, for example, with IR radiation or by means of microwave radiation. The heating results in destruction of the layer formed by substance D, for example by melting or at least partial destruction of substance D. This measure is of interest especially when heating is to result in release of the active, e.g. the release of an aroma in the case of a heating pad.

Measure 6) is of interest especially for compositions where release of the active is not desired until use of the composition comprising the microparticles laden with the active. This may be, for example, in the case of microparticles comprising an aroma, clothing, or else cosmetics that come into contact with body heat or body fluids. Measure 6 is alternatively of interest in the case of crop protection formulations comprising microparticles laden with the active, and where release in the soil or on the plant is desired.

The present invention further provides compositions of the microparticles filled with at least one active, obtainable by the process of the invention. The compositions of the invention preferably comprise the active in a total amount of 5% to 95% by weight, based on the total weight of the microparticles laden with the actives, i.e. of the constituents of the composition other than solvents. The constituents of the microparticles, i.e. the constituents of the composition other than solvents, are essentially the active, the polymer that forms the wall material, the solidified substances A, B, C and D, and any auxiliaries that are used in the production of the microparticles or in the loading of the microparticles and are not removed. The compositions of the invention may be in the form either of a suspension or of a powder, preference being given to powders.

The present invention further provides products comprising a composition of the invention. Preference is given to products comprising the compositions of the invention in a proportion by weight of 0.01% to 80% by weight, based on the total weight of the product.

The nature of the product is naturally guided by the nature of the active and may be a product which typically comprises an aroma, for example a perfume, a washing product, a cleaning product, a cosmetic product, a personal care product, a hygiene article, a food, a food supplement or a fragrance dispenser. The product may alternatively be a pharmaceutical product, a crop protection product or an additive intended for use in building materials.

The present invention further provides for the use of compositions of the invention in the aforementioned products. Preference is given to the use of compositions of the invention in a product selected from perfumes, washing products, cleaning products, cosmetic products, personal care products, hygiene articles, foods, food supplements, fragrance dispensers and fragrances.

Compositions of the invention that comprise a fragrance as active may be used in the production of perfumed articles. The olfactory properties and also the physical properties and the non-toxicity of the compositions of the invention underline their particular suitability for the end uses mentioned.

The use of the compositions is found to be particularly advantageous in conjunction with top notes of compositions, by way of example in perfume compositions comprising dihydrorosan, rose oxides or other readily volatile fragrances, e.g. isoamyl acetate, prenyl acetate or methylheptenone. In this case, the release of the important, sought-after top notes is effectively delayed. The fragrance or aroma compositions are accordingly metered in at the suitable point in the requisite amount. In the mint compositions of L-menthol, DL-menthol, L-menthone and L-menthyl acetate described, aside from the aroma effect, a cooling effect is additionally applied in a targeted manner, e.g. in chewing gums, confectionery, cosmetic products, and industrial applications such as in textiles or superabsorbents. A further advantage lies in the high material compatibility of the compositions of the invention even with reactive or comparatively unstable components such as aldehydes, esters, pyrans/ethers, which may exhibit secondary reactions on the surfaces.

The positive properties contribute to use of the compositions of the invention with particular preference in perfume products, personal care products, hygiene articles, textile detergents and in cleaning products for solid surfaces.

The perfumed article is selected, for example, from perfume products, personal care products, hygiene articles, textile detergents and cleaning products for solid surfaces. Preferred perfumed articles of the invention are also selected from:

perfume products selected from perfume extracts, eau de parfums, eau de toilettes, eau de colognes, eau de solide, extrait parfum, air fresheners in liquid form, gel form or a form applied to a solid carrier, aerosol sprays, scented cleaners and scented oils;
personal care products selected from aftershaves, pre-shave products, splash colognes, solid and liquid soaps, shower gels, shampoos, shaving soaps, shaving foams, bath oils, cosmetic emulsions of the oil-in-water type, of the water-in-oil type and of the water-in-oil-in-water type, for example skin creams and lotions, face creams and lotions, sunscreen creams and lotions, aftersun creams and lotions, hand creams and lotions, foot creams and lotions, hair removal creams and lotions, aftershave creams and lotions, tanning creams and lotions, hair care products, for example hairsprays, hair gels, setting hair lotions, hair conditioners, hair shampoo, permanent and semipermanent hair colorants, hair shaping compositions such as cold waves and hair smoothing compositions, hair tonics, hair creams and hair lotions, deodorants and antiperspirants, for example underarm sprays, roll-ons, deodorant sticks, deodorant creams, products of decorative cosmetics, for example eye shadows, nail varnishes, make-ups, lipsticks, mascara, toothpaste, dental floss;
hygiene articles selected from candles, lamp oils, joss sticks, propellants, rust removers, perfumed freshening wipes, armpit pads, baby diapers, sanitary towels, toilet paper, cosmetic wipes, pocket tissues, dishwasher deodorizer;
cleaning products for solid surfaces selected from perfumed acidic, alkaline and neutral cleaning products, for example floor cleaners, window cleaners, dishwashing detergents, bath and sanitary cleaners, scouring milk, solid and liquid toilet cleaners, powder and foam carpet cleaners, waxes and polishes such as furniture polishes, floor waxes, shoe creams, disinfectants, surface disinfectants and sanitary cleaners, brake cleaners, pipe cleaners, limescale removers, grill and oven cleaners, algae and moss removers, mold removers, facade cleaners;
textile detergents selected from liquid detergents, powder detergents, laundry pretreatments such as bleaches, soaking agents and stain removers, fabric softeners, washing soaps, washing tablets.

In a further aspect, the compositions of the invention are suitable for use in surfactant-containing perfumed articles. This is because there is frequently a search—especially for the perfuming of surfactant-containing formulations, for example cleaning products (in particular dishwashing compositions and all-purpose cleaners)—for odorants and/or odorant compositions with a rose top note and marked naturalness.

In a further aspect, the compositions of the invention can be used as products for providing (a) hair or (b) textile fibers with a rosy odor note.

The compositions of the invention are therefore of particularly good suitability for use in surfactant-containing perfumed articles.

It is preferred if the perfumed article is one of the following:
an acidic, alkaline or neutral cleaning product selected in particular from the group consisting of all-purpose cleaners, floor cleaners, window cleaners, dishwashing detergents, bath and sanitary cleaners, scouring milk, solid and liquid toilet cleaners, powder and foam carpet cleaners, liquid detergents, powder detergents, laundry pretreatments such as bleaches, soaking agents and stain removers, fabric softeners, washing soaps, washing tablets, disinfectants, surface disinfectants,
an air freshener in liquid form, gel-like form or a form applied to a solid carrier or as an aerosol spray,
a wax or a polish selected in particular from the group consisting of furniture polishes, floor waxes and shoe creams, or
a personal care product selected in particular from the group consisting of shower gels and shampoos, shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, aftersun creams and lotions, hand creams and lotions, foot creams and lotions, hair removal creams and lotions, aftershave creams and lotions, tanning creams and lotions, hair care products such as e.g. hairsprays, hair gels, setting hair lotions, hair conditioners, permanent and semipermanent hair colorants, hair shaping compositions such as cold waves and hair smoothing compositions, hair tonics, hair creams and hair lotions, deodorants and antiperspirants such as e.g. underarm sprays, roll-ons, deodorant sticks, deodorant creams, products of decorative cosmetics.

The customary ingredients with which odorants used in accordance with the invention, or odorant compositions of the invention, may be combined are common knowledge and are described for example in WO 2016/050836, the teaching of which is hereby expressly incorporated by reference.

Preference is likewise given to the use of compositions of the invention for controlled release of actives such as crop protecting agents and pharmaceutical agents.

### FIGURES

FIG. 1 shows a scanning electron micrograph of the microparticles from example 1.

FIG. 2 shows a scanning electron micrograph of the microparticles from production example 1.

FIG. 3 shows a scanning electron micrograph of the microparticles from example 5.

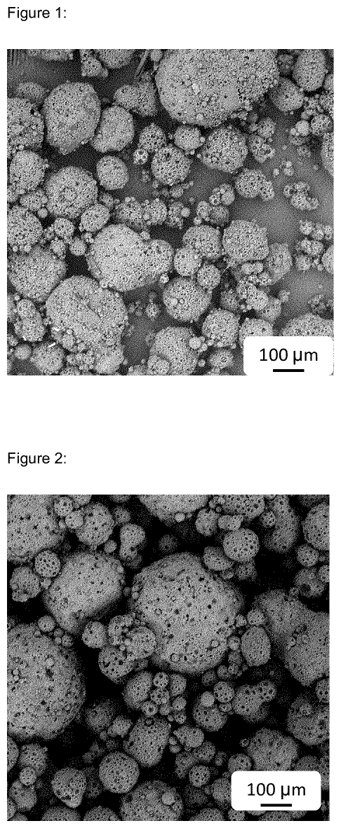

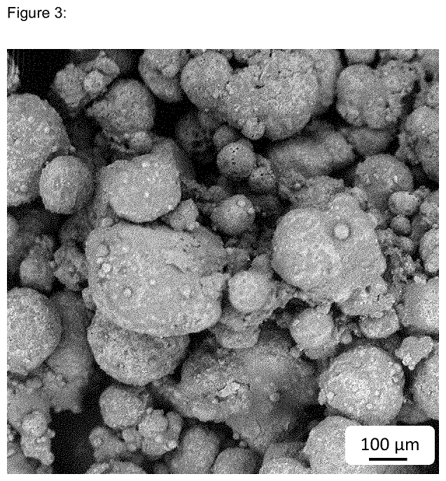

### EXAMPLES

Materials

Unless stated otherwise, the following materials and components were used:
polybutylene sebacate terephthalate (PBSeT): Ecoflex™ FS Blend A1300 product from BASF SE
polybutylene succinate adipinate (PBSA): BioPBS™ FD92 product from MCPP Germany GmbH
polylactic acid (PLA)
polycaprolactone (PCL) Capa6506 product from Perstorp
polyethylene glycol (PEG9000): Pluriol E 9000 from BASF SE
aroma chemical mixture: A water-immiscible fragrance mixture having a fruity, pearlike note and characterized by the following evaporation rate at 25° C. and 1 bar:

| | Aroma chemical mixture |
|---|---|
| Time [hours] | Δ M [%]* |
| 0 | 0 |
| 3 | 8 |
| 5 | 10 |
| 94 | 46 |
| 145 | 51 |
| 170 | 54 |
| 190 | 57 |
| 480 | 69 |

*Decrease in mass of the aroma chemical mixture in % by weight normalized to the starting value

beeswax: melting point 63° C., density of 0.96 to 0.964 g/mL at 15° C. (Fisher Scientific)
triglyceride mixture: Myritol® 318 from BASF SE
polyvinyl alcohol: degree of hydrolysis 88 mol %, viscosity 25 mPa*s (4% strength aqueous solution at 20° C.), proportion of carboxyl groups 3 mol %

### Methods

### Determining the Average Particle Diameter in Aqueous Suspension/Emulsion by Light Scattering

The particle diameter of the w/o/w emulsion or the particle suspension is determined with a Malvern Mastersizer 2000 from Malvern Instruments, England, Hydro 2000S sample dispersion unit, by a standard test method documented in the literature. The value D[4,3] is the volume-weighted average.

### Determining the Average Particle Diameter of the Solid

The microparticles are determined as powder with a Malvern Mastersizer 2000 from Malvern Instruments, England, including Scirocco 2000 powder feed unit, by a standard test method documented in the literature. The value D[4,3] is the volume-weighted average.

### Production Example 1: Production of Spherical Finable Microparticles

Spherical finable microparticles were produced analogously to example 8 of WO 2018/065481. The matrix-forming polymer used was a polymer blend of 70% by weight of polybutylene sebacate terephthalate (PBSeT; Ecoflex™ FS Blend A1300 product from BASF SE) and 30% by weight of polylactic acid (PLA). The procedure was as follows:

Pore former solution: 0.54 kg of ammonium carbonate was dissolved in 53.5 kg of water (pore former).
Solution of the aliphatic-aromatic polyester: 15.1 kg of PBSeT and 6.5 kg of PLA were stirred into 270.0 kg of dichloromethane and dissolved at 25° C. while stirring.

The w/o emulsion was produced by emulsifying the pore former solution in the solution of the aliphatic-aromatic polyester at 170 rpm with a twin-level cross-beam stirrer for 15 minutes.

The w/o emulsion thus created was transferred into 423 kg of a 0.8% strength by weight, aqueous polyvinyl alcohol solution and likewise emulsified with shear and energy input (one minute at 120 rpm with an impeller stirrer).

Stirring of the w/o/w emulsion thus created was then continued with an impeller stirrer at 120 rpm, while reducing the pressure to 800 mbar and gradually increasing the jacket temperature to 40° C. and keeping it at this temperature for 4 hours. Thereafter, the microparticle suspension was cooled to room temperature, filtered and dried at 37° C.

The average particle diameter D[4,3], determined from the aqueous suspension, was 220 μm. Bulk density was determined to DIN EN ISO 60:1999 and was 0.15 g/cm³. The pore size was 8.5 μm and was determined by means of mercury porosimetry. Visual evaluation was likewise effected and showed an average pore size at the surface of 7 μm.

### Production Example 2: Production of Spherical Finable Microparticles

Spherical tillable microparticles were produced analogously to example 8 of WO 2018/065481. The matrix-forming polymer used was a polymer blend of 70% by weight of PBSeT and 30% by weight of PBSA. The procedure was as follows:

Pore former solution: 0.54 kg of ammonium carbonate was dissolved in 53.5 kg of water (pore former).
Solution of the aliphatic-aromatic polyester: 15.1 kg of PBSeT and 6.5 kg of PBSA were stirred into 270.0 kg of dichloromethane and dissolved at 25° C. while stirring.

The w/o emulsion was produced by emulsifying the pore former solution in the solution of the aliphatic-aromatic polyester at 170 rpm with a twin-level cross-beam stirrer for 15 minutes.

The w/o emulsion thus created was transferred into 423 kg of a 2.4% strength by weight, aqueous polyvinyl alcohol solution and likewise emulsified with shear and energy input (one minute at 120 rpm with a round anchor stirrer).

Stirring of the w/o/w emulsion thus created was then continued with an impeller stirrer at 120 rpm. In the process, the pressure was reduced to 800 mbar and the jacket temperature gradually increased to 40° C. and it was kept at this temperature for 4 hours. Thereafter, the microparticle suspension was cooled to room temperature, filtered and dried at 37° C.

The average particle diameter D[4,3], determined from the aqueous suspension, was 130 μm.

### Production Example 3

Spherical finable microparticles were produced analogously to example 8 of WO 2018/065481. The matrix-forming polymer used was a polymer blend of 70% by weight of PBSeT and 30% by weight of PCL. The procedure was as follows:

Pore former solution: 0.54 kg of ammonium carbonate was dissolved in 53.5 kg of water (pore former).
Solution of the aliphatic-aromatic polyester: 15.1 kg of PBSeT and 6.5 kg of PCL were stirred into 270.0 kg of dichloromethane and dissolved at 25° C. while stirring.

The w/o emulsion was produced by emulsifying the pore former solution in the solution of the aliphatic-aromatic polyester at 170 rpm with a twin-level cross-beam stirrer for 15 minutes.

The w/o emulsion thus created was transferred into 423 kg of a 0.8% strength by weight aqueous polyvinyl alcohol solution and likewise emulsified with shear and energy input (one minute at 120 rpm with a round anchor stirrer).

Stirring of the w/o/w emulsion thus created was then continued with an impeller stirrer at 120 rpm. In the process, the pressure was reduced to 800 mbar and the jacket temperature was gradually increased to 40° C. and it was kept at this temperature for 4 hours. Thereafter, the microparticle suspension was cooled to room temperature, filtered and dried at 37° C.

The average particle diameter D[4,3], determined from the aqueous suspension, was 110 μm.

### Example 1

A homogeneous mixture of beeswax and an aroma chemical mixture was produced by melting 5.0 g of beeswax in a water bath at 85° C. and adding 5.0 g of the aroma chemical mixture.

0.29 g of the melt produced was dripped onto 0.29 g of the tillable spherical microparticles from example 1 while stirring. Then the formulation was cooled to room temperature.

### Comparative Example 1

As a reference sample, microparticles from production example 1 were filled with the aroma chemical mixture in analogy to the method of example 1, but without substance A. The quantitative ratios of microparticles to aroma chemicals were chosen so as to obtain the same mass ratio between aroma chemical mixture and spherically tillable microparticles as in example 1. For this purpose, while stirring, 0.15 g of the aroma chemical mixture from example 1 was dripped onto 0.29 g of the tillable spherical microparticles from production example 1 while stirring.

### Study of Storage Stability

The microparticle compositions from example 1 and comparative example 1 were stored together at 25° C. and a relative air humidity of 50% in a climate-controlled cabinet. The decrease in mass of the aroma chemical mixture was determined via the decrease in weight of the sample. The results are summarized in Table 1.

TABLE 1

| | Example 1 | Comparative example 1 |
|---|---|---|
| Time [hours] | Δ M [%]* | Δ M [%]* |
| 0 | 0 | 0 |
| 30 | 11 | 25 |
| 46 | 12 | 33 |
| 74 | 26 | 41 |
| 145 | 30 | 40 |
| 170 | 34 | 54 |
| 480 | 42 | 69 |
| 650 | 43 | 73 |
| 1180 | 57 | 82 |

*Decrease in mass of the sample in % by weight normalized to weight of the aroma chemical mixture

### Example 2

A homogeneous mixture of polyethylene glycol (PEG9000) and the aroma chemical mixture was produced by melting 5.0 g of PEG9000 in a water bath at 85° C. and adding 5.0 g of the aroma chemical mixture. 0.29 g of the melt thus produced was dripped onto 0.29 g of the tillable spherical microparticles from example 1 while stirring. Then the composition thus obtained was cooled to room temperature.

The microparticle composition from example 2 was stored together with the microparticle composition from comparative example 1 at 25° C. and a relative air humidity of 50% in a climate-controlled cabinet. The decrease in mass of the aroma chemical mixture was determined via the decrease in weight of the sample. The results are summarized in Table 2.

TABLE 2

| | Example 2 | Comparative example 1 |
|---|---|---|
| Time [hours] | Δ M [%]* | Δ M [%]* |
| 0 | 0 | 0 |
| 25 | 10 | 23 |
| 70 | 21 | 40 |
| 140 | 32 | 51 |
| 190 | 38 | 56 |
| 240 | 40 | 58 |
| 500 | 52 | 69 |
| 670 | 53 | 73 |

*Decrease in mass of the sample in % by weight normalized to weight of the aroma chemical mixture

### Example 3

5.0 g of the microparticles from production example 1 were mixed with 20.0 g of the aroma chemical mixture on a roll mixer for five hours. Subsequently, the suspension was filtered, and the filtercake was rinsed three times with 10% by weight aqueous propanediol solution and then dried at room temperature overnight. 0.97 g of the filled microparticles thus obtained, comprising the aroma chemical mixture in an amount of 60%, based on the total weight of the particles, was removed and stirred with a melt of 2.23 g of beeswax, so as to form a wax film on the microparticles. The mixture was cooled and stored.

### Example 4

5.0 g of the microparticles from production example 1 were mixed with 20.0 g of the aroma chemical mixture on a roll mixer for five hours. Subsequently, the suspension was filtered, and the filtercake was rinsed three times with 10% by weight aqueous propanediol solution and then dried at room temperature overnight. 0.14 g of the filled microparticles thus obtained, comprising the aroma chemical mixture in an amount of 60%, based on the total weight of the particles, was removed and stirred in a melt of 1.34 g of carnauba wax (melting range from 82 to 86° C.), so as to form a wax film on the microparticles. The mixture was cooled and stored.

### Comparative Example 2

5.0 g of the microparticles from production example 1 were mixed with 20.0 g of the aroma chemical mixture on a roll mixer for five hours. Subsequently, the suspension was filtered, and the filtercake was rinsed three times with 10% by weight aqueous propanediol solution and then dried at room temperature overnight. The filled microparticles thus obtained, comprising the aroma chemical mixture in an amount of 60%, based on the total weight of the particles, were removed and used to examine storage stability.

The microparticle compositions from examples 3 and 4 were stored together with the microparticle composition from comparative example 2 at 25° C. and a relative air humidity of 50% in a climate-controlled cabinet. The decrease in mass of the aroma chemical mixture was determined via the decrease in weight of the sample. The reference examined was the decrease in mass of the pure, unencapsulated aroma chemical. The results are summarized in Table 3.

TABLE 3

| | Example 3 | Example 4 | Comparative example 2 |
|---|---|---|---|
| Time [hours] | Δ M [%]* | Δ M [%]* | Δ M [%]* |
| 0 | 0 | 0 | 0 |
| 3 | 3 | n.d. | 4 |
| 5 | 8 | 8 | 20 |
| 94 | 36 | 29 | 38 |
| 145 | 39 | 39 | 56 |
| 170 | 41 | 43 | 59 |
| 190 | 42 | 49 | 71 |
| 260 | 47 | 56 | 79 |

*Decrease in mass of the sample in % by weight normalized to weight of the aroma chemical mixture

### Examples 5a-5f

a) 500 g of the microparticles from production example 1 were initially charged in a ploughshare mixer and sprayed with 1000 g of a triglyceride mixture at 20° C. by means of a single-substance nozzle having a nozzle diameter of 0.5 mm (spray pressure 2 bar) within 2 mins (flow rate 500 ml/min). 400 g of the filled microparticles thus obtained were again initially charged in a ploughshare mixer and sprayed by means of a single-substance nozzle (spray pressure 4 bar, flow rate 100 ml/min) at 70° C. with 100 g of a beeswax melt (heated to 75° C.) within 10 min, so as to form a wax film on the microparticles. The microparticles thus obtained were cooled and discharged from the mixer.
b) Example 5a can be conducted in the same way with a mixture of the aroma mixture with the triglyceride mixture.
c) Example 5a can be conducted in the same way with the pure aroma mixture.
d) Example 5a can be conducted in the same way with a 10% by weight solution of L-menthol in propane-1,2-diol.
e) Example 5a can be conducted in the same way with a 10% by weight solution of rose oxide in propane-1,2-diol.
f) Example 5a can be conducted in the same way with a 10% by weight solution of Dihydrorosan in propane-1,2-diol.

### Examples 6a-6f

a) 500 g of the microparticles from production example 2 were initially charged in a plowshare mixer. To this was added dropwise 1000 g of a triglyceride mixture by means of a funnel at 20° C. within 5 min. 400 g of the filled microparticles thus obtained were in turn initially charged in a plowshare mixer and sprayed by means of a one-phase nozzle (spray pressure 4 bar, flow rate 100 ml/min) with 100 g of a beeswax melt (at a temperature of 75° C.) at 70° C. within 10 min, such that a wax film forms on the microparticles. The microparticles thus obtained were cooled down and discharged from the mixer.
b) Example 6a can be conducted in the same way with a mixture of the aroma mixture with the triglyceride mixture.
c) Example 6a can be conducted in the same way with the pure aroma mixture.
d) Example 6a can be conducted in the same way with a 10% by weight solution of L-menthol in propane-1,2-diol.
e) Example 6a can be conducted in the same way with a 10% by weight solution of rose oxide in propane-1,2-diol.
f) Example 6a can be conducted in the same way with a 10% by weight solution of Dihydrorosan in propane-1,2-diol.

### Examples 7a-f

a) 500 g of the microparticles from production example 3 were sprayed with 1000 g of a triglyceride mixture by the method of example 5a. 400 g of the filled microparticles thus obtained were in turn initially charged in a plowshare mixer and sprayed with 100 g of a beeswax melt (at a temperature of 75° C.) by means of a one-phase nozzle (spray pressure 4 bar, flow rate 100 ml/min) at 70° C. within 10 min, so as to form a wax film on the microparticles. The microparticles thus obtained were cooled down and discharged from the mixer.
b) Example 7a can be conducted in the same way with a mixture of the aroma mixture with the triglyceride mixture.
c) Example 7a can be conducted in the same way with the pure aroma mixture.
d) Example 7a can be conducted in the same way with a 10% by weight solution of L-menthol in propane-1,2-diol.
e) Example 7a can be conducted in the same way with a 10% by weight solution of rose oxide in propane-1,2-diol.
f) Example 7a can be conducted in the same way with a 10% by weight solution of Dihydrorosan in propane-1,2-diol.

### Example 8

500 g of the microparticles from production example 1 were sprayed by the method of example 5a with 1000 g of a mint aroma mixture consisting of 2.3% by weight of L-isopulegol, 3.1% by weight of L-methyl acetate, 36.4% by weight of L-menthone and 58.2% by weight of L-menthol. 400 g of the filled microparticles thus obtained were in turn initially charged in a plowshare mixer and sprayed with 100 g of a beeswax melt (at a temperature of 75° C.) by means of a one-phase nozzle (spray pressure 4 bar, flow rate 100 ml/min) at 70° C. within 10 min, so as to form a wax film on the microparticles. The microparticles thus obtained were cooled down and discharged from the mixer.

### Example 9

In this example, a mixture of aroma chemicals having comparable evaporation characteristics to the aroma chemical mixture used in the other examples but a different fruity, apple-like note was used.

200.0 g of the aroma chemical mixture were then applied dropwise while stirring to 100.0 g of the microparticles from production example 1. 0.30 g of the filled microparticles thus obtained was initially charged in an aluminum dish and sprayed with 0.8 g of a mixture of a tetra(ethylene glycol) diacrylate and the photoinitiator 2-hydroxy-2-methyl-1-phenyl-1-propanone in a mass ratio of 98:2. Subsequently, the microparticles thus treated were irradiated with a LED-UV source, Bluepoint LED eco from Dr. Hönle AG, at a power of 80% of the maximum power, which led to immediate curing of the tetra(ethylene glycol) diacrylate.

The microparticle composition thus obtained was stored in a climate-controlled cabinet together with the unencapsulated aroma chemical mixture at 25° C. and a relative air humidity of 50%. The decrease in mass of the aroma chemical mixture was determined via the decrease in weight of the sample. The results are compiled in table 4.

TABLE 4

| | Example 9 | Aroma chemical |
|---|---|---|
| Time [hours] | Δ M [%]* | Δ M [%]* |
| 0 | 0 | 0 |
| 7 | 0 | 24 |
| 13 | 3 | 33 |
| 36 | 13 | 45 |

*decrease in mass of the sample in % by weight, normalized to weight of the aroma chemical mixture

1.-49. (canceled)

50. A process for producing microparticles laden with at least one organic active of low molecular weight, wherein the microparticles have been formed from an organic, polymeric wall material and in the unladen state, in their interior, have at least one cavity connected via pores to the surface of the microparticles, wherein
(a) the unladen microparticles are impregnated with a liquid (1a) consisting essentially of
i) the active, in molten, emulsified, suspended or dissolved form in the liquid,
ii) at least one nonpolymerizable substance A which is solid at room temperature and is in molten, emulsified, suspended or dissolved form in the liquid, and
iii) optionally one or more solvents;
or
(b) the unladen microparticles are impregnated with a liquid (1b) consisting essentially of
i) the active, in molten, emulsified, suspended or dissolved form in the liquid,
ii) at least one polymerizable substance B in emulsified or dissolved form in the liquid,
iii) optionally a nonpolymerizable substance A which is solid at room temperature and is in molten, emulsified, suspended or dissolved form in the liquid, and
iv) optionally one or more solvents;
and then polymerization of substance B is brought about;
or
(c) the unladen microparticles are impregnated with a liquid (1c) consisting essentially of
i) the active, in molten, emulsified, suspended or dissolved form in the liquid,
ii) at least one substance C which is in dissolved or molten form in the liquid and can be solidified by addition of polyvalent ions,
iii) optionally a nonpolymerizable substance A which is solid at room temperature and is in molten, emulsified, suspended or dissolved form in the liquid, and
iv) optionally one or more solvents;
and then a solution of polyvalent ions is added in order to bring about solidification of substance C.

51. A process for producing microparticles laden with at least one organic active of low molecular weight, wherein the microparticles have been formed from an organic, polymeric wall material and in the unladen state, in their interior, have at least one cavity connected via pores to the surface of the microparticles, wherein
d1) the unladen microparticles are impregnated with a liquid (1d) comprising the active, and then
d2) substance that seals the pores of the laden microparticles is applied to the surface of the laden microparticles.

52. A process for producing microparticles laden with at least one organic active of low molecular weight, wherein the microparticles have been formed from an organic, polymeric wall material and in the unladen state, in their interior, have at least one cavity connected via pores to the surface of the microparticles, wherein the unladen microparticles are impregnated with a liquid comprising the active by applying the liquid in finely divided form to the unladen microparticles.

53. The process according to claim 50, wherein the nonpolymerizable substance A is selected from the group consisting of organic polymers that melt at a temperature in the range from 30 to 150° C., organic polymers that are solubilizable in any solvent present, and waxes, and mixtures thereof.

54. The process according to claim 50, wherein the liquid (1a) used is a melt or solution consisting essentially of at least one active and at least one organic polymer and/or at least one wax, wherein the wax or organic polymer is in molten form or in the form of a solution in the active in the liquid.

55. The process according to claim 53, wherein the nonpolymerizable substance A is selected from the group consisting of vegetable or animal waxes, polyalkylene glycols, water-solubilizable polymers, and mixtures thereof.

56. The process according to claim 54, wherein the liquid (1a) used is a mixture of an aqueous solution or emulsion of the water-solubilizable polymer and the active.

57. The process according to claim 50, wherein the mass ratio of the at least one active to the nonpolymerizable substance A in the liquid (1a) is in the range from 99:1 to 10:90 and the mass ratio of the at least one active to the polymerizable substance B in the liquid (1b) is in the range from 99:1 to 10:90.

58. The process according to claim 50, wherein the polymerizable substance B is selected from the group consisting of ethylenically unsaturated monomers, silanes having hydroxyl or alkoxy groups, and oxidatively polymerizable aromatic compounds.

59. The process according to claim 50, wherein the liquid (1b) used is an emulsion or solution consisting essentially of at least one active and at least one polymerizable substance B, wherein the polymerizable substance B is in molten form or in the form of a solution in the active in the liquid.

60. The process according to claim 50, wherein the treating of the microparticles with the liquid (1a), (1b), (1c) or (1d) is accomplished by using the microparticles in the form of a powder.

61. The process according to claim 50, wherein the impregnating of the microparticles with the liquid (1a), (1b), (1c) or (1d) is accomplished by spray application or dropwise application of the respective liquid onto the microparticles or suspension of the microparticles in the respective liquid.

62. The process according to claim 51, wherein a solid coating is produced on the surface of the microparticles.

63. The process according to claim 62, wherein the microparticles are treated with a liquid (2d) comprising
i) at least one film-forming substance D in molten, emulsified, dispersed or dissolved form in the liquid, and
ii) optionally one or more solvents,
in such a way as to form a solid coating on the surface of the microparticles.

64. The process according to claim 63, wherein the film-forming substance D is selected from the group consisting of organic polymers that melt at a temperature in the range from 30 to 150° C., organic polymers that are solubilizable and/or dispersible in any solvent present in the liquid (2d), vegetable or animal waxes, polyalkylene glycols, homo- and copolymers of vinyl acetate, water-solubilizable and/or water dispersible polymers, and mixtures thereof.

65. The process according to claim 64, wherein the liquid (2d) used is
1) a melt or solution consisting essentially of at least one organic polymer and/or at least one wax, wherein the wax or organic polymer is in molten form or in the form of a solution, dispersion or emulsion in the solvent in the liquid or
2) a solution, dispersion or emulsion of the water-solubilizable and/or water-dispersible polymer.

66. The process according to claim 65, wherein the film-forming substance D is selected from a polymerizable substance selected from the group consisting of ethylenically unsaturated monomers, silanes having hydroxyl or alkoxy groups, and oxidatively polymerizable aromatic compounds, and the film formation comprises polymerization of substance D.

67. The process according to claim 65, wherein the liquid (2d) is used in such an amount that the mass ratio of the microparticles obtained in step (d1) to substance D present in the liquid (2d) is in the range from 95:5 to 20:80.

68. The process according to claim 65, wherein a coating is produced on the surface of the microparticles by powdering the microparticles with a finely divided solid and then bringing about film formation on the surface of the microparticles or a coating is produced on the surface of the microparticles by depositing a volatile substance from the gas phase on the surface of the microparticles and converting it to a solid from the surface by chemical reaction.

69. The process according to claim 51, wherein step (d2) is conducted in such a way that the thickness of the coating obtained averages in the range from 0.01 to 1.5 times the average radius of the microparticles.

70. The process according to claim 50, wherein the impregnating of the microparticles with the liquid (1a), (1b), (1c) or (1d) is accomplished using a composition of microparticles in which the microparticles, prior to the filling, have an average particle diameter of 10 to 600 μm, wherein at least 80% of those microparticles that have a particle diameter that differs from the average particle diameter of the microparticles in the composition by not more than 20% each have an average of at least 10 pores having a diameter in the range from 1/5000 to ⅕ of the average particle diameter, and, in addition, the diameter of each of these pores is at least 20 nm.

71. The process according to claim 50, wherein the polymeric wall material comprises at least one aliphatic-aromatic polyester which is an ester of an aliphatic dihydroxy compound esterified with a composition of aromatic dicarboxylic acid and aliphatic dicarboxylic acid.

72. The process according to claim 50, wherein the wall material, besides the aliphatic-aromatic polyester, additionally comprises at least one further polymer that is different from aliphatic-aromatic polyesters and is selected from the group consisting of polymerized hydroxycarboxylic acids, aliphatic-aliphatic polyesters, polylactones, poly(p-dioxanones), polyanhydrides, polyesteramides, polylactic acid, aliphatic poly-C₅-C₁₂-lactones, aliphatic-aliphatic polyesters, and polyhydroxy fatty acids.

73. The process according to claim 50, wherein the active is liquid at 22° C. and 1013 mbar or has a melting point below 100° C.

74. The process according to claim 50, wherein the active is selected from the group consisting of aroma chemicals, organic crop protecting agents, organic pharmaceutical agents, cosmetic actives, and actives for construction chemical applications.
